(19) United States
(12) Patent Application Publication
(10) Pub. No.: US 2022/0273666 A1
(43) Pub. Date: Sep. 1, 2022

(54) NANOPARTICLE FORMULATION OF BCL-2 INHIBITOR

(71) Applicant: Recurium IP Holdings, LLC, San Diego, CA (US)

(72) Inventors: Joseph Robert Pinchman, San Diego, CA (US); Aditya Krishnan Unni, San Diego, CA (US); Yosef Shamay, San Diego, CA (US); Kevin Duane Bunker, Escondido, CA (US); Peter Qinhua Huang, San Diego, CA (US)

(21) Appl. No.: 17/597,471

(22) PCT Filed: Jul. 8, 2020
(86) PCT No.: PCT/US2020/041168

§ 371 Date: Jan. 6, 2022

Related U.S. Application Data
(60) Provisional application No. 62/872,565, filed on Jul. 10, 2019.

(51) Int. Cl. A61K 31/5377; A61K 31/496; A61K 9/51; A61K 47/64

(52) U.S. Cl. CPC A61K 31/5377; A61K 31/496; B82Y 5/00; A61K 47/643; A61K 9/5169

(57) ABSTRACT

Various albumin nanoparticle Bcl-2 inhibitor formulations are described, along with methods of using them to treat conditions characterized by excessive cellular proliferation, such as cancer and tumors. In various embodiments, such Bcl-2 inhibitor formulations contain albumin and a compound of the following Formula (I), or a pharmaceutically acceptable salt thereof, where the variables in Formula (I) are defined herein.

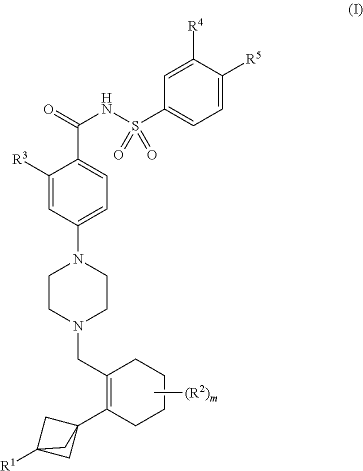

### INCORPORATION BY REFERENCE TO ANY PRIORITY APPLICATIONS

This application claims priority to U.S. Provisional Application Ser. No. 62/872,565, filed Jul. 10, 2019, which is hereby incorporated by reference in its entirety.

### BACKGROUND

### Field

This application relates to albumin nanoparticle Bcl-2 inhibitor formulations and methods of using them to treat conditions characterized by excessive cellular proliferation, such as cancer and tumors.

Description of the Field

Proteins in the Bcl-2 family contain Bcl-2 homology (BH) domains and regulate apoptosis by modulating mitochondrial outer membrane permeabilization (MOMP). Members of the Bcl-2 family have up to four BH domains, referred to as BH1, BH2, BH3 and BH4. All four domains are conserved in the anti-apoptotic Bcl-2 family members Bcl-2, Bcl-xL, Bcl-W, Mcl-1 and A1/Bfl-1.

A number of compounds that inhibit anti-apoptotic Bcl-2 proteins have been evaluated for their ability to treat lymphomas and other types of cancer. Navitoclax, a dual Bcl-2/xL inhibitor, has been evaluated in Phase I/II clinical trials for the treatment of chronic lymphocytic leukemia (CLL). However, its efficacy in the study population was reduced by dosage limitations due to the occurrence of thrombocytopenia, a known side effect of inhibiting Bcl-xL.

Venetoclax is the first Bcl-2 inhibitor approved by the FDA. It is available commercially from AbbVie Inc. under the tradename VENCLEXTA. It is currently indicated as a second line treatment for patients with CLL or small lymphocytic lymphoma (SLL). According to the VENCLEXTA label, it is supplied to the patient in the form of 10 mg, 50 mg and 100 mg tablets that are administered orally in accordance with the following 5-week ramp-up dosing schedule:

| Week | Daily Dose |
|---|---|
| 1 | 20 mg |
| 2 | 50 mg |
| 3 | 100 mg |
| 4 | 200 mg |
| 5 and beyond | 400 mg |

Maximum plasma concentration of venetoclax was reached 5 to 8 hours following multiple oral administration under fed conditions. Patients are instructed to take VENCLEXTA tablets with a meal and water at approximately the same time each day. VENCLEXTA tablets should be swallowed whole and not chewed, crushed, or broken prior to swallowing.

The development of such VENCLEXTA oral formulations represents a substantial advance in the art of formulating Bcl-2 inhibitors. However, there remains a need for improved formulations of inhibitors in the Bcl-2 family that can improve tolerability, exposure, efficacy, and overcome dose limiting toxicities.

### SUMMARY

Some embodiments described herein relate to a pharmaceutical composition that can include an effective amount of one or more of compounds of Formula (I), or a pharmaceutically acceptable salt thereof, and a pharmaceutically acceptable carrier comprising albumin. In various embodiments, the compound of Formula (I) and the albumin in such pharmaceutical compositions are formulated as particles.

Some embodiments described herein relate to a method for treating a cancer or a tumor described herein that can include administering an effective amount of such a pharmaceutical composition to a subject having a cancer described herein. Other embodiments described herein relate to the use of such a pharmaceutical composition in the manufacture of a medicament for treating a cancer or a tumor described herein. Still other embodiments described herein relate to an effective amount of such a pharmaceutical composition for treating a cancer or a tumor described herein.

Some embodiments described herein relate to a method for inhibiting replication of a malignant growth or a tumor described herein that can include contacting the growth or the tumor with an effective amount of such a pharmaceutical composition as described herein. Other embodiments described herein relate to the use of an effective amount of such a pharmaceutical composition in the manufacture of a medicament for inhibiting replication of a malignant growth or a tumor described herein. Still other embodiments described herein relate to an effective amount of such a pharmaceutical composition for inhibiting replication of a malignant growth or a tumor described herein.

Some embodiments described herein relate to a method for treating a cancer described herein that can include contacting a malignant growth or a tumor described herein with an effective amount of such a pharmaceutical composition described herein. Other embodiments described herein relate to the use of an effective amount of such a pharmaceutical composition in the manufacture of a medicament for treating a cancer described herein, wherein the use comprises contacting a malignant growth or a tumor described herein with the medicament. Still other embodiments described herein relate to the use of an effective amount of such a pharmaceutical composition for contacting a malignant growth or a tumor described herein, wherein the malignant growth or tumor is due to a cancer described herein.

Some embodiments described herein relate to a method for inhibiting the activity of Bcl-2 that can include administering an effective amount of a pharmaceutical composition as described herein to a subject and can also include contacting a cell that expresses Bcl-2 with an effective amount of such a pharmaceutical composition. Other embodiments described herein relate to the use of an effective amount of such a pharmaceutical composition in the manufacture of a medicament for inhibiting the activity of Bcl-2 in a subject or, in the manufacture of a medicament for inhibiting the activity of Bcl-2, wherein the use comprises contacting a cell that expresses Bcl-2. Still other embodiments described herein relate to an effective amount of such a pharmaceutical composition for inhibiting the activity of Bcl-2 in a subject; or for inhibiting the activity of Bcl-2 by contacting a cell that expresses Bcl-2.

These and other embodiments are described in greater detail below.

### BRIEF DESCRIPTION OF THE DRAWINGS

FIG. 1 shows examples of compounds of the Formula (I).

FIG. 2 shows examples of compounds of the Formula (I).

### DETAILED DESCRIPTION

Bcl-2 is a critical regulator of programmed cell death (apoptosis). Bcl-2 belongs to the B cell lymphoma 2 (BCL-2) family of proteins, which includes both pro-apoptotic proteins (such as Bak, Bax, Bim, Bid, tBid, Bad, Bik, PUMA, Bnip-1, Hrk, Bmf and Noxa) and anti-apoptotic proteins (such as Bcl-2, Bcl-X_{L}, Bcl-W, Mcl-1 and Bcl-2A1). For example, under normal conditions, Bcl-2 inhibits apoptosis in part by preventing activation of Bak and Bax. Activation of the intrinsic apoptosis pathway (e.g., by cellular stress) inhibits Bcl-2, thus activating Bak and Bax. These proteins facilitate mitochondrial outer membrane permeabilization, releasing cytochrome c and Smac. This initiates the caspase signaling pathway, ultimately resulting in cell death. Dysregulation of Bcl-2 leads to sequestration of cell-death-promoting proteins, leading to evasion of apoptosis. This process contributes to malignancy, and facilitates cell survival under other disadvantageous conditions, such as during viral infection. Inhibition of Bcl-2 (e.g., by degrading Bcl-2 protein and/or by inhibiting binding) disrupts sequestration of pro-apoptotic proteins, restoring apoptotic signaling, and promoting damaged cells to undergo programmed cell death. Therefore, inhibition of proteins in the Bcl-2 family (e.g., by inhibition and/or degradation of Bcl-2 protein and/or Bcl-X_{L} protein) has the potential to ameliorate or treat cancers and tumors.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, 0-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl) and a di-substituted amine(alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

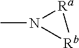

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

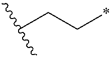 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 4 carbon atoms. An alkylene group may be substituted or unsubstituted. For example, a lower alkylene group can be substituted by replacing one or more hydrogen of the lower alkylene group and/or by substituting both hydrogens on the same carbon with a C₂₋₆ monocyclic cycloalkyl group

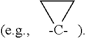

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro[3.3]heptane and spiro[4.5]decane.

As used herein, “cycloalkenyl” refers to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “aryl” refers to a carbocyclic (all carbon) monocyclic or multicyclic (such as bicyclic) aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic (such as bicyclic) aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R” group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The terms “amino” and “unsubstituted amino” as used herein refer to a —NH₂ group.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. A mono-substituted amine group can include, for example, a mono-alkylamine group, a mono-C₁-C₆ alkylamine group, a mono-arylamine group, a mono-C₆-C₁₀ arylamine group and the like. Examples of mono-substituted amine groups include, but are not limited to, —NH(methyl), —NH(phenyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. A di-substituted amine group can include, for example, a di-alkylamine group, a di-C₁-C₆ alkylamine group, a di-arylamine group, a di-C₆-C₁₀ arylamine group and the like. Examples of di-substituted amine groups include, but are not limited to, —N(methyl)₂, —N(phenyl)(methyl), —N(ethyl)(methyl) and the like.

As used herein, “mono-substituted amine(alkyl)” group refers to a mono-substituted amine as provided herein connected, as a substituent, via a lower alkylene group. A mono-substituted amine(alkyl) may be substituted or unsubstituted. A mono-substituted amine(alkyl) group can include, for example, a mono-alkylamine(alkyl) group, a mono-C₁-C₆ alkylamine(C₁-C₆ alkyl) group, a mono-arylamine(alkyl group), a mono-C₆-C₁₀ arylamine(C₁-C₆ alkyl) group and the like. Examples of mono-substituted amine(alkyl) groups include, but are not limited to, —CH₂NH(methyl), —CH₂NH(phenyl), —CH₂CH₂NH(methyl), —CH₂CH₂NH(phenyl) and the like.

As used herein, “di-substituted amine(alkyl)” group refers to a di-substituted amine as provided herein connected, as a substituent, via a lower alkylene group. A di-substituted amine(alkyl) may be substituted or unsubstituted. A di-substituted amine(alkyl) group can include, for example, a dialkylamine(alkyl) group, a di-C₁-C₆ alkylamine(C₁-C₆ alkyl) group, a di-arylamine(alkyl) group, a di-C₆-C₁₀ arylamine(C₁-C₆ alkyl) group and the like. Examples of di-substituted amine(alkyl)groups include, but are not limited to, —CH₂N(methyl)₂, —CH₂N(phenyl)(methyl), —NCH₂(ethyl)(methyl), —CH₂CH₂N(methyl)₂, —CH₂CH₂N(phenyl)(methyl), —NCH₂CH₂(ethyl)(methyl) and the like.

Where the number of substituents is not specified (e.g. haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (I), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

The term “Bcl protein inhibitor” refers to an agent (including small molecules and proteins) that inhibit the binding of an anti-apoptic Bcl protein (such as Bcl-2, Bcl-X_{L}, Bcl-W, Mcl-1 and Bcl-2A1) to a pro-apoptotic Bcl protein (such as Bak, Bax, Bim, Bid, tBid, Bad, Bik, PUMA, Bnip-1, Hrk, Bmf and Noxa). Bcl protein inhibitors include, but are not limited to venetoclax, navitoclax, obatoclax, 555746, APG-2575, ABT-737, AMG176, AZD5991 and APG-1252. Additional Bcl protein inhibitors include, but are not limited to, compounds disclosed in PCT Application Publication Nos. WO2017/132474, WO 2014/113413 and WO 2013/110890, U.S. Patent Application Publication No. 2015/0051189 and Chinese Patent Application No. CN 106565607, which are each incorporated herein by reference for the limited purpose of disclosing additional Bcl protein inhibitors. As will be understood by those of skill in the art, there are numerous methods of evaluating protein binding interactions, including, but not limited to co-immunoprecipitation, fluorescence resonance energy transfer (FRET), surface plasmon resonance (SPR) and fluorescence polarization/anisotropy.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments described herein relate to a pharmaceutical composition comprising a compound of Formula (I), or a pharmaceutically acceptable salt thereof, and a pharmaceutically acceptable carrier comprising albumin, wherein the compound of Formula (I) has the structure:

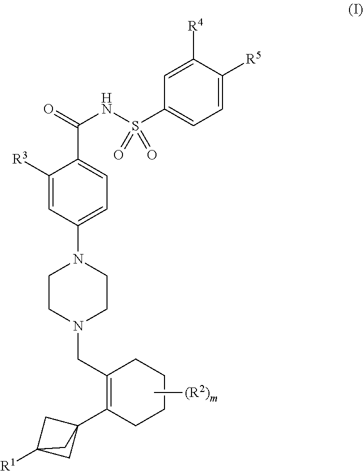

wherein: R¹ can be selected from hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamine and an unsubstituted di-C₁-C₆ alkylamine; each R² can be independently selected from halogen, a substituted or unsubstituted C₁-C₆ alkyl, a substituted or unsubstituted C₁-C₆ haloalkyl and a substituted or unsubstituted C₃-C₆ cycloalkyl; R³ can be selected from hydrogen, halogen, X—R^{3A},

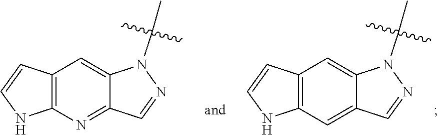 [SMILES: *n1ncc2nc3[nH]ccc3cc21 ; *n1ncc2cc3[nH]ccc3cc21]

R^{3A} can be a substituted or unsubstituted 5 to 10 membered heteroaryl; R⁴ can be selected from NO₂, S(O)R⁶, SO₂R⁶, halogen, cyano and an unsubstituted C₁-C₆ haloalkyl; R⁵ can be selected from —X¹-(Alk¹)ₙ-R⁷ and —X²(CHR⁸)-(Alk²)ₚ-X³-R⁹; Alk¹ and Alk² can be independently selected from an unsubstituted C₁-C₄ alkylene and a C₁-C₄ alkylene substituted with 1, 2 or 3 substituents independently selected from fluoro, chloro, an unsubstituted C₁-C₃ alkyl and an unsubstituted C₁-C₃ haloalkyl; R⁶ can be selected from a substituted or unsubstituted C₁-C₆ alkyl, a substituted or unsubstituted C₁-C₆ haloalkyl and a substituted or unsubstituted C₃-C₆ cycloalkyl; R⁷ can be selected from a substituted or unsubstituted C₁-C₆ alkoxy, a substituted or unsubstituted C₃-C₁₀ cycloalkyl, a substituted or unsubstituted 3 to 10 membered heterocyclyl, hydroxy, an amino group, a substituted or unsubstituted mono-substituted amine group, a substituted or unsubstituted di-substituted amine group, a substituted or unsubstituted N-carbamyl, a substituted or unsubstituted C-amido and a substituted or unsubstituted N-amido; R⁸ can be selected from a substituted or unsubstituted 3 to 10 membered heterocyclyl(C₁-C₆ alkyl), a substituted or unsubstituted di-C₁-C₆ alkylamine(C₁-C₆ alkyl) and a substituted or unsubstituted mono-C₁-C₆ alkylamine(C₁-C₆ alkyl); R⁹ can be selected from a substituted or unsubstituted 5 to 10 membered heteroaryl and a substituted or unsubstituted monocyclic or bicyclic C₆-C₁₀ aryl; m can be 0, 1, 2 and 3; n and p can be independently selected from 0 and 1; X, X¹, X² and X³ can be independently selected from —O—, —S— and —NH—; and wherein when m is 2 or 3, two R² groups can be taken together with the atom(s) to which they are attached to form a substituted or unsubstituted C₃-C₆ cycloalkyl or a substituted or unsubstituted 3 to 6 membered heterocyclyl.

In some embodiments, R¹ can be halogen, for example, fluoro, chloro, bromo or iodo. In some embodiments, R¹ can be fluoro. In some embodiments, R¹ can be chloro. In some embodiments, R¹ can be hydrogen.

In some embodiments, R¹ can be a substituted or unsubstituted C₁-C₆ alkyl. For example, in some embodiments, R¹ can be a substituted C₁-C₆ alkyl. In other embodiments, R¹ can be an unsubstituted C₁-C₆ alkyl. Examples of suitable C₁-C₆ alkyl groups include, but are not limited to methyl, ethyl, n-propyl, isopropyl, n-butyl, isobutyl, tert-butyl, pentyl (branched and straight-chained) and hexyl (branched and straight-chained). In some embodiments, R¹ can be an unsubstituted methyl or an unsubstituted ethyl.

In some embodiments, R¹ can be a substituted or unsubstituted C₁-C₆ haloalkyl, for example, a substituted or unsubstituted mono-halo C₁-C₆ alkyl, a substituted or unsubstituted di-halo C₁-C₆ alkyl, a substituted or unsubstituted tri-halo C₁-C₆ alkyl, a substituted or unsubstituted tetra-halo C₁-C₆ alkyl or a substituted or unsubstituted penta-halo C₁-C₆ alkyl. In some embodiments, R¹ can be an unsubstituted —CHF₂, —CF₃, —CH₂CF₃, —CF₂CH₃ or —CF₂CF₃.

In some embodiments, R¹ can be a substituted or unsubstituted monocyclic or bicyclic C₃-C₆ cycloalkyl. For example, in some embodiments, R¹ can be a substituted monocyclic C₃-C₆ cycloalkyl. In other embodiments, R¹ can be an unsubstituted monocyclic C₃-C₆ cycloalkyl. Examples of suitable monocyclic or bicyclic C₃-C₆ cycloalkyl groups include, but are not limited to cyclopropyl, cyclobutyl, cyclopentyl, [1.1.1]bicyclopentyl and cyclohexyl.

In some embodiments, R¹ can be a substituted or unsubstituted C₁-C₆ alkoxy. For example, in some embodiments, R¹ can be a substituted C₁-C₆ alkoxy. In other embodiments, R¹ can be an unsubstituted C₁-C₆ alkoxy. Examples of suitable C₁-C₆ alkoxy groups include, but are not limited to methoxy, ethoxy, n-propoxy, isopropoxy, n-butoxy, isobutoxy, tert-butoxy, pentoxy (branched and straight-chained) and hexoxy (branched and straight-chained). In some embodiments, R¹ can be an unsubstituted methoxy or an unsubstituted ethoxy.

In some embodiments, R¹ can be an unsubstituted mono-C₁-C₆ alkylamine, for example, methylamine, ethylamine, n-propylamine, isopropylamine, n-butylamine, isobutylamine, tert-butylamine, pentylamine (branched and straight-chained) and hexylamine (branched and straight-chained). In some embodiments, R¹ can be methylamine or ethylamine.

In some embodiments, R¹ can be an unsubstituted di-C₁-C₆ alkylamine. In some embodiments, each C₁-C₆ alkyl in the di-C₁-C₆ alkylamine is the same. In other embodiments, each C₁-C₆ alkyl in the di-C₁-C₆ alkylamine is different. Examples of suitable di-C₁-C₆ alkylamine groups include, but are not limited to di-methylamine, di-ethylamine, (methyl)(ethyl)amine, (methyl)(isopropyl)amine and (ethyl)(isopropyl)amine.

In some embodiments, m can be 0. When m is 0, those skilled in the art understand that the ring to which R² is attached is unsubstituted. In some embodiments, m can be 1. In some embodiments, m can be 2. In some embodiments, m can be 3.

In some embodiments, one R² can be an unsubstituted C₁-C₆ alkyl (for example, methyl, ethyl, n-propyl, isopropyl, n-butyl, isobutyl, tert-butyl, pentyl (branched and straight-chained) and hexyl (branched and straight-chained)) and any other R², if present, can be independently selected from halogen (for example, fluoro or chloro), a substituted or unsubstituted C₁-C₆ alkyl (such as those described herein), a substituted or unsubstituted C₁-C₆haloalkyl (such as those described herein) and a substituted or unsubstituted monocyclic or bicyclic C₃-C₆ cycloalkyl (such as those described herein). In some embodiments, each R² can be independently selected from an unsubstituted C₁-C₆ alkyl, such as those described herein.

In some embodiments, m can be 2; and each R² can be geminal. In some embodiments, m can be 2; and each R² can be vicinal. In some embodiments, m can be 2; and each R² can be an unsubstituted methyl. In some embodiments, m can be 2; and each R² can be a geminal unsubstituted methyl.

In some embodiments, two R² groups can be taken together with the atom(s) to which they are attached to form a substituted or unsubstituted monocyclic C₃-C₆ cycloalkyl. For example, in some embodiments, two R² groups can be taken together with the atom(s) to which they are attached to form a substituted monocyclic C₃-C₆ cycloalkyl, such as those described herein. In other embodiments, two R² groups can be taken together with the atom(s) to which they are attached to form an unsubstituted monocyclic C₃-C₆ cycloalkyl, such as those described herein. In some embodiments, two R² groups can be taken together with the atom to which they are attached to form an unsubstituted cyclopropyl or an unsubstituted cyclobutyl.

In some embodiments, two R² groups can be taken together with the atom(s) to which they are attached to form a substituted or unsubstituted monocyclic 3 to 6 membered heterocyclyl. For example, in some embodiments, two R² groups can be taken together with the atom(s) to which they are attached to form a substituted monocyclic 3 to 6 membered heterocyclyl. In other embodiments, two R² groups can be taken together with the atom(s) to which they are attached to form an unsubstituted monocyclic 3 to 6 membered monocyclic heterocyclyl. In some embodiments, the substituted monocyclic 3 to 6 membered heterocyclyl can be substituted on one or more nitrogen atoms. Examples of suitable substituted or unsubstituted monocyclic 3 to 6 membered heterocyclyl groups include, but are not limited to azidirine, oxirane, azetidine, oxetane, pyrrolidine, tetrahydrofuran, imidazoline, pyrazolidine, piperidine, tetrahydropyran, piperazine, morpholine, thiomorpholine and dioxane.

In some embodiments, R³ can be

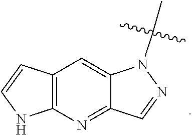 [SMILES: *n1ncc2nc3[nH]ccc3cc21]

In some embodiments, R³ can be

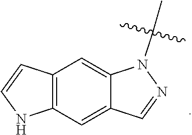 [SMILES: *n1ncc2cc3[nH]ccc3cc21]

In some embodiments, R³ can be X—R^{3A}. In some embodiments, X can be —O—. In some embodiments, X can be —S—. In some embodiments, X can be —NH—. In some embodiments, R^{3A} can be

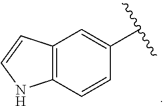 [SMILES: *c1ccc2[nH]ccc2c1]

In some embodiments, R^{3A} can be

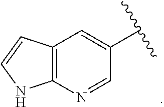 [SMILES: *c1cnc2[nH]ccc2c1]

In some embodiments, R^{3A} can be a substituted or unsubstituted 5 to 10 membered heteroaryl. In some embodiments, R^{3A} can be a substituted 5 to 10 membered monocyclic heteroaryl. In other embodiments, R^{3A} can be a substituted 5 to 10 membered bicyclic heteroaryl. In some embodiments, R^{3A} can be an unsubstituted 5 to 10 membered monocyclic heteroaryl. In other embodiments, R^{3A} can be an unsubstituted 5 to 10 membered bicyclic heteroaryl. Examples of suitable substituted or unsubstituted monocyclic or bicyclic 5 to 10 membered heteroaryl groups include, but are not limited to pyrrole, furan, thiophene, imidazole, pyrazole, oxazole, isoxazole, thiazole, isothiazole, triazole, pyridine, pyridazine, pyrimidine, pyrazine, pyrrolo-pyrroles, pyrrolo-furans, pyrrolo-thiophenes, indole, isoindole, indolizine, indazole, benzimidazole, azaindoles, azaindazoles, purine, benzofuran, isobenzofuran, benzothiophene, isobenzothiophene, quinoline, isoquinoline, quinoxaline, phthalazine, quinazoline, cinnoline, 1,8-naphthyridine, pyrido-pyrimidines and pteridine.

In some embodiments, R³ can be hydrogen. In some embodiments, R³ can be halogen. In some embodiments, R³ can be fluoro or chloro.

In some embodiments, R⁴ can be NO₂. In some embodiments, R⁴ can be cyano. In some embodiments, R⁴ can be halogen.

In some embodiments, R⁴ can be an unsubstituted C₁-C₆ haloalkyl, such as those described herein. In some embodiments, R⁴ can be —CF₃.

In some embodiments, R⁴ can be S(O)R⁶. In some embodiments, R⁴ can be SO₂R⁶. In some embodiments, R⁴ can be SO₂CF₃.

In some embodiments, R⁶ can be a substituted or unsubstituted C₁-C₆ alkyl. For example, in some embodiments, R⁶ can be a substituted C₁-C₆ alkyl, such as those described herein. In other embodiments, R⁶ can be an unsubstituted C₁-C₆ alkyl, such as those described herein.

In some embodiments, R⁶ can be a substituted or unsubstituted monocyclic or bicyclic C₃-C₆ cycloalkyl. For example, in some embodiments, R⁶ can be a substituted monocyclic or bicyclic C₃-C₆ cycloalkyl. In other embodiments, R⁶ can be an unsubstituted monocyclic or bicyclic C₃-C₆ cycloalkyl. Examples of suitable monocyclic or bicyclic C₃-C₆ cycloalkyl groups include, but are not limited to cyclopropyl, cyclobutyl, cyclopentyl, [1.1.1]bicyclopentyl and cyclohexyl.

In some embodiments, R⁶ can be a substituted or unsubstituted C₁-C₆ haloalkyl, such as those described herein. In some embodiments, R⁶ can be —CF₃.

In some embodiments, R⁵ can be —X¹-(Alk¹)ₙ-R⁷. In some embodiments, X¹ can be —O—. In some embodiments, X¹ can be —S—. In some embodiments, X¹ can be —NH—.

In some embodiments, Alk¹ can be unsubstituted —(CH₂)₁₋₄—* for which “*” represents the point of attachment to R⁷. In some embodiments, Alk¹ can be

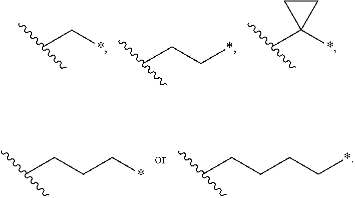

In some embodiments, Alk¹ can be a substituted

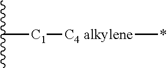

for which “*” represents the point of attachment to R⁷. For example, in some embodiments, Alk¹ can be a substituted methylene, a substituted ethylene, a substituted propylene or a substituted butylene. In some embodiments, Alk¹ can be mono-substituted, di-substituted or tri-substituted. In some embodiments, Alk¹ can be mono-substituted with a halogen (such as fluoro or chloro) or unsubstituted C₁-C₃ alkyl, such as those described herein. In other embodiments, Alk¹ can be mono-substituted unsubstituted C₁-C₃ haloalkyl, such as those described herein. In some embodiments, Alk¹ can be mono-substituted with fluoro or unsubstituted methyl. In some embodiments, Alk¹ can be di-substituted with one fluoro and one unsubstituted C₁-C₃ alkyl, such as those described herein. In other embodiments, Alk¹ can be di-substituted with one unsubstituted C₁-C₃ haloalkyl, such as those described herein, and one unsubstituted C₁-C₃ alkyl, such as those described herein. In some embodiments, Alk¹ can be di-substituted with one fluoro and one unsubstituted methyl. In some embodiments, Alk¹ can be di-substituted with two independently selected unsubstituted C₁-C₃alkyl groups, such as those described herein. In some embodiments, Alk¹ can be di-substituted with unsubstituted methyl.

In some embodiments, Alk¹ can be selected from:

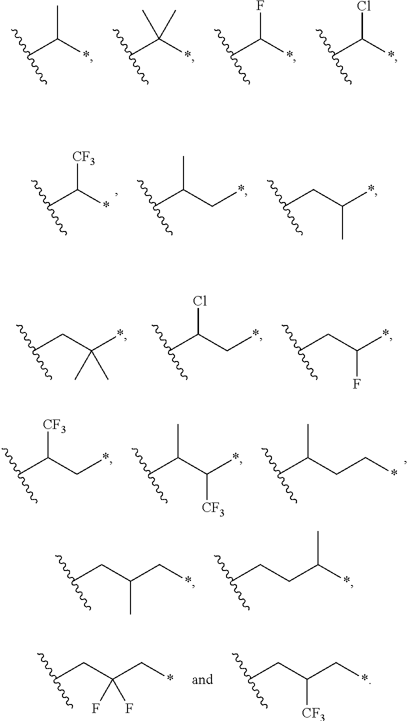

In some embodiments, n can be 0. When n is 0, those skilled in the art understand that X¹ is directly connected to R⁷. In some embodiments, n can be 1.

In some embodiments, R⁷ can be a substituted or unsubstituted mono-substituted amine group. For example, R⁷ can be an amino group mono-substituted with a substituted or unsubstituted C₁-C₆ alkyl, a substituted or unsubstituted C₂-C₆ alkenyl, a substituted or unsubstituted C₂-C₆ alkynyl, a substituted or unsubstituted monocyclic or bicyclic C₃-C₆ cycloalkyl, a substituted or unsubstituted monocyclic or bicyclic C₆-C₁₀ aryl, a substituted or unsubstituted monocyclic or bicyclic 5 to 10 membered heteroaryl, a substituted or unsubstituted monocyclic or bicyclic 3 to 10 membered heterocyclyl, a substituted or unsubstituted monocyclic or bicyclic C₃-C₆ cycloalkyl(unsubstituted C₁-C₆ alkyl), a substituted or unsubstituted monocyclic or bicyclic C₆-C₁₀ aryl(unsubstituted C₁-C₆ alkyl), a substituted or unsubstituted monocyclic or bicyclic 5 to 10 membered heteroaryl(unsubstituted C₁-C₆ alkyl) or a substituted or unsubstituted monocyclic or bicyclic 3 to 10 membered heterocyclyl(unsubstituted C₁-C₆ alkyl). Examples of suitable mono-substituted amine groups include, but are not limited to —NH(methyl), —NH(isopropyl), —NH(cyclopropyl), —NH(phenyl), —NH(benzyl) and —NH(pyridine-3-yl).

In some embodiments, R⁷ can be a substituted or unsubstituted di-substituted amine group. For example, R⁷ can be an amino group substituted with two substituents independently selected from a substituted or unsubstituted C₁-C₆ alkyl, a substituted or unsubstituted C₂-C₆ alkenyl, a substituted or unsubstituted C₂-C₆ alkynyl, a substituted or unsubstituted monocyclic or bicyclic C₃-C₆ cycloalkyl, a substituted or unsubstituted monocyclic or bicyclic C₆-C₁₀ aryl, a substituted or unsubstituted monocyclic or bicyclic 5 to 10 membered heteroaryl, a substituted or unsubstituted monocyclic or bicyclic 3 to 10 membered heterocyclyl, a substituted or unsubstituted monocyclic or bicyclic C₃-C₆ cycloalkyl(unsubstituted C₁-C₆ alkyl), a substituted or unsubstituted monocyclic or bicyclic C₆-C₁₀ aryl(unsubstituted C₁-C₆ alkyl), a substituted or unsubstituted monocyclic or bicyclic 5 to 10 membered heteroaryl(unsubstituted C₁-C₆ alkyl) or a substituted or unsubstituted monocyclic or bicyclic 3 to 10 membered heterocyclyl(unsubstituted C₁-C₆ alkyl). In some embodiments the two substituents can be the same. In other embodiments the two substituents can be different. Examples of suitable di-substituted amine groups include, but are not limited to, —N(methyl)₂, —N(ethyl)₂, —N(isopropyl)₂, —N(benzyl)₂, —N(ethyl)(methyl), —N(isopropyl)(methyl), —N(ethyl)(isopropyl), —N(phenyl)(methyl) and —N(benzyl)(methyl).

In some embodiments, R⁷ can be selected from a substituted or unsubstituted N-carbamyl, a substituted or unsubstituted C-amido and a substituted or unsubstituted N-amido.

In some embodiments, R⁷ can be a substituted or unsubstituted C₃-C₁₀ cycloalkyl. In some embodiments, R⁷ can be a substituted or unsubstituted monocyclic C₃-C₁₀ cycloalkyl. In other embodiments, R⁷ can be a substituted or unsubstituted bicyclic C₃-C₁₀ cycloalkyl, for example, a bridged, fused or spiro C₃-C₁₀ cycloalkyl. Suitable substituted or unsubstituted monocyclic or bicyclic C₃-C₁₀ cycloalkyl groups include, but are not limited to, cyclopropyl, cyclobutyl, cyclopentyl, cyclohexyl, cycloheptyl, cyclooctyl, cyclononyl, cyclodecyl, spiro[3.3]heptyl, spiro[2.3]hexyl, spiro[3.4]octyl, spiro[3.5]nonyl, spiro[3.6]decyl, spiro[2.4]heptyl, spiro[4.4]nonyl, spiro[4.5]decyl, spiro[2.5]octyl, spiro[3.5]nonyl, bicyclo[1.1.1]pentyl, bicyclo[2.1.1]hexyl, bicyclo[2.2.1]heptyl, decahydronaphthalenyl, octahydro-1H-indenyl, octahydropentalenyl, bicyclo[4.2.0]octyl, bicyclo[2.1.0]pentyl and bicyclo[3.2.0]heptyl.

In some embodiments, R⁷ can be a substituted or unsubstituted C₆-C₁₀ spirocycloalkyl. In some embodiments, R⁷ can be a substituted C₆-C₁₀ spirocycloalkyl. In other embodiments, R⁷ can be an unsubstituted C₆-C₁₀ spirocycloalkyl. In some embodiments, R⁷ can be a substituted or unsubstituted -cyclopropyl-cyclobutyl spiroalkyl, -cyclopropyl-cyclopentyl spiroalkyl, -cyclopropyl-cyclohexyl spiroalkyl, -cyclopropyl-cycloheptyl spiroalkyl, -cyclopropyl-cyclooctyl spiroalkyl, -cyclobutyl-cyclopropyl spiroalkyl, -cyclobutyl-cyclobutyl spiroalkyl, -cyclobutyl-cyclopentyl spiroalkyl, -cyclobutyl-cyclohexyl spiroalkyl, -cyclobutyl-cycloheptyl spiroalkyl, -cyclopentyl-cyclopropyl spiroalkyl, -cyclopentyl-cyclobutyl spiroalkyl, -cyclopentyl-cyclopentyl spiroalkyl, cyclopentyl-cyclohexyl spiroalkyl, -cyclohexyl-cyclopropyl spiroalkyl, -cyclohexyl-cyclobutyl spiroalkyl, -cyclohexyl-cyclopentyl spiroalkyl, -cycloheptyl-cyclopropyl spiroalkyl, -cycloheptyl-cyclobutyl spiroalkyl or -cyclooctyl-cyclopropyl spiroalkyl.

In some embodiments, R⁷ can be a substituted or unsubstituted 3 to 10 membered heterocyclyl. In some embodiments, R⁷ can be a substituted 3 to 10 membered heterocyclyl. In other embodiments, R⁷ can be an unsubstituted 3 to 10 membered heterocyclyl. In some embodiments, R⁷ can be a substituted or unsubstituted monocyclic 3 to 10 membered heterocyclyl. In other embodiments, R⁷ can be a substituted or unsubstituted bicyclic 5 to 10 membered heterocyclyl, for example, a fused, bridged or spiro 5 to 10 membered heterocyclyl. Suitable substituted or unsubstituted 3 to 10 membered heterocyclyl groups include, but are not limited to, azidirine, oxirane, azetidine, oxetane, pyrrolidine, tetrahydrofuran, imidazoline, pyrazolidine, piperidine, tetrahydropyran, piperazine, morpholine, thiomorpholine, dioxane, 2-azaspiro[3.3]heptane, 2-oxaspiro[3.3]heptane, 2,6-diazaspiro[3.3]heptane, 2-oxa-6-azaspiro[3.3]heptane, 2-azaspiro[3.4]octane, 6-oxaspiro[3.4]octane, 6-oxa-2-azaspiro[3.4]octane, 7-oxa-2-azaspiro[3.5]nonane, 7-oxaspiro[3.5]nonane and 2-oxa-8-azaspiro[4.5]decane. In some embodiments, the substituted or unsubstituted monocyclic or bicyclic 3 to 10 membered heterocyclyl can be connected to the rest of the molecule through a nitrogen atom. In other embodiments, the substituted or unsubstituted monocyclic or bicyclic 3 to 10 membered heterocyclyl can be connected to the rest of the molecule through a carbon atom. In some embodiments, the substituted monocyclic or bicyclic 3 to 10 membered heterocyclyl can be substituted on one or more nitrogen atoms.

In some embodiments, R⁷ can be a substituted or unsubstituted 6 to 10 membered spiro heterocyclyl. In some embodiments, R⁷ can be a substituted 6 to 10 membered spiro heterocyclyl. In other embodiments, R⁷ can be an unsubstituted 6 to 10 membered spiro heterocyclyl. In some embodiments, R⁷ can be a substituted or unsubstituted azaspirohexane, azaspiroheptane, azaspirooctane, oxaspirohexane, oxaspiroheptane, oxaspirooctane, diazaspirohexane, diazaspiroheptane, diazaspirooctane, dioxaspirohexane, dioxaspiroheptane, dioxaspirooctane, oxa-azaspirohexane, oxa-azaspiroheptane or oxa-azaspirooctane. Suitable substituted or unsubstituted 3 to 10 membered heterocyclyl groups include, but are not limited to, 2-azaspiro[3.3]heptane, 2-oxaspiro[3.3]heptane, 2,6-diazaspiro[3.3]heptane, 2-oxa-6-azaspiro[3.3]heptane, 2-azaspiro[3.4]octane, 6-oxaspiro[3.4]octane, 6-oxa-2-azaspiro[3.4]octane, 7-oxa-2-azaspiro[3.5]nonane, 7-oxaspiro[3.5]nonane and 2-oxa-8-azaspiro[4.5]decane. In some embodiments, the substituted or unsubstituted 6 to 10 membered spiro heterocyclyl can be connected to the rest of the molecule through a nitrogen atom. In other embodiments, the substituted or unsubstituted 6 to 10 membered spiro heterocyclyl can be connected to the rest of the molecule through a carbon atom. In some embodiments, the substituted 6 to 10 membered spiroheterocyclyl can be substituted on one or more nitrogen atoms.

In some embodiments, R⁷ can be hydroxy or amino.

In some embodiments, R⁷ can be unsubstituted. In other embodiments, R⁷ can be substituted. In some embodiments, R⁷ can be substituted with 1 or 2 substituents independently selected from an unsubstituted C₁-C₆ alkyl (such as those described herein), an unsubstituted C₁-C₆ alkoxy (such as those described herein), fluoro, chloro, hydroxy and —SO₂-(unsubstituted C₁-C₆ alkyl). For example, the C₁-C₆ alkoxy, C₃-C₁₀ cycloalkyl, 3 to 10 membered heterocyclyl, mono-substituted amine group, di-substituted amine group, N-carbamyl, C-amido and N-amido groups of R⁷ can be substituted with 1 or 2 substituents independently selected from any of the aforementioned substituents.

In some embodiments, R⁷ can be

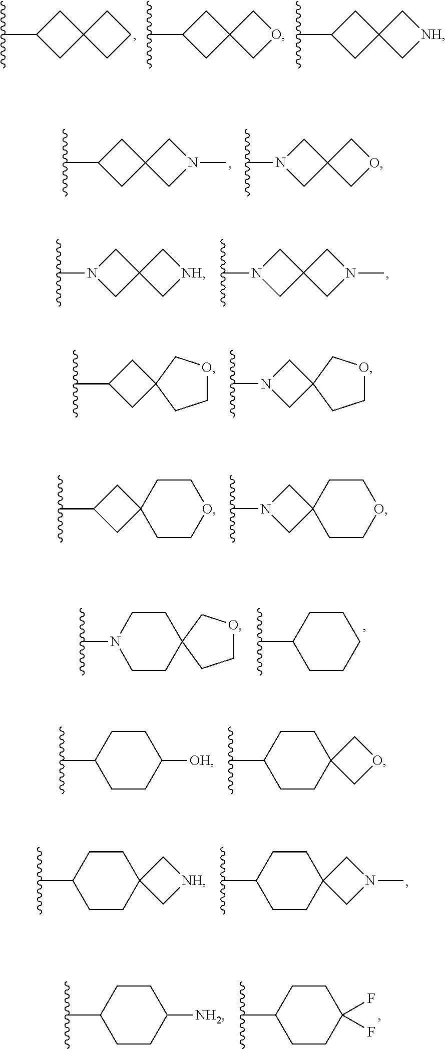

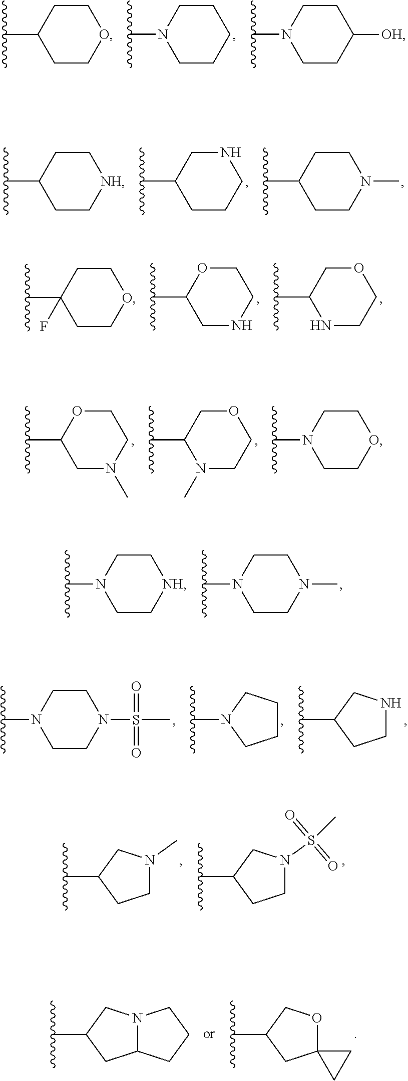

In some embodiments, R⁷ can be

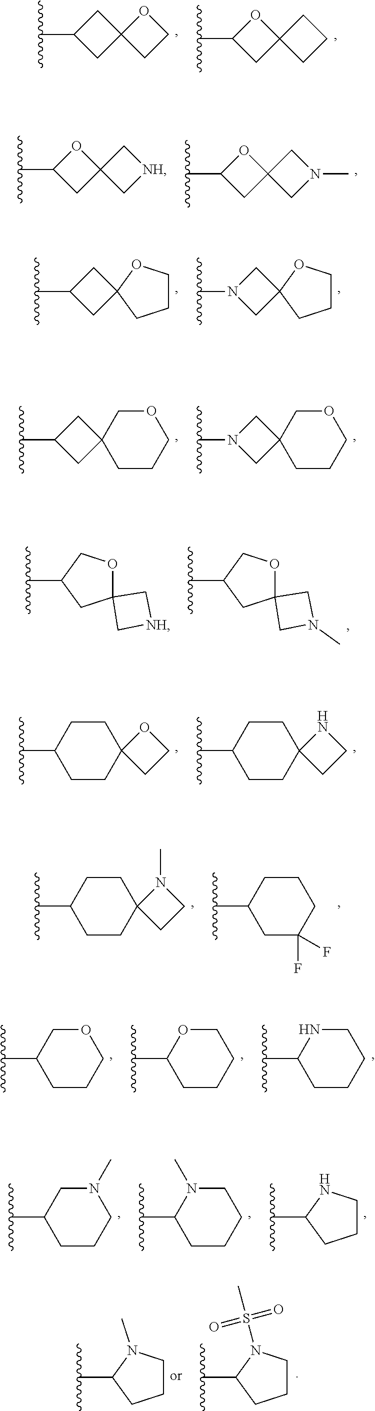

In some embodiments, R⁷ can be

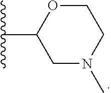 [SMILES: *C1CN(C)CCO1]

For example, in some embodiments R⁷ can be

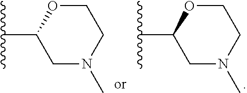

In some embodiments R⁷ can be

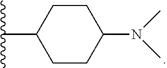 [SMILES: *C1CCC(N(C)C)CC1]

For example, in some embodiments R⁷ can be

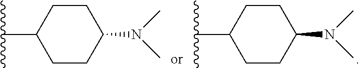

In some embodiments R⁷ can be

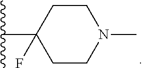 [SMILES: *C1(F)CCN(C)CC1]

In some embodiments R⁷ can be

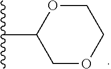 [SMILES: *C1COCCO1]

For example, in some embodiments R⁷ can be

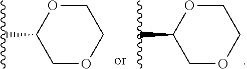

In some embodiments R⁷ can be

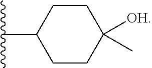 [SMILES: *C1CCC(C)(O)CC1]

For example, in some embodiments R⁷ can be

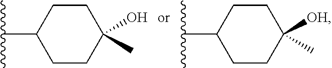

such as

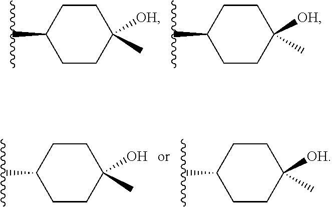

In some embodiments, R⁵ can be —X²—(CHR⁸)-(Alk²)ₚ-X³-R⁹. In some embodiments, X² can be —O—. In some embodiments, X² can be —S—. In some embodiments, X² can be —NH—. In some embodiments, X³ can be —O—. In some embodiments, X³ can be —S—. In some embodiments, X³ can be —NH—. In some embodiments, X² can be —NH— and X³ can be —S—. In some embodiments, X² can be —O— and X³ can be —S—. In some embodiments, X² can be —NH— and X³ can be —O—. In some embodiments, X² can be —O— and X³ can be —O—.

In some embodiments, Alk² can be unsubstituted —(CH₂)₁₋₄—* for which “*” represents the point of attachment to X³. In some embodiments, Alk² can be an unsubstituted methylene, an unsubstituted ethylene, an unsubstituted propylene or an unsubstituted butylene. In some embodiments, Alk² can be * or

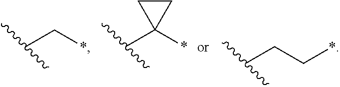

In some embodiments, Alk² can be a substituted

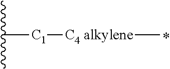

for which “*” represents the point of attachment to X³. In some embodiments, Alk² can be a substituted methylene, a substituted ethylene, a substituted propylene or a substituted butylene. In some embodiments, Alk² can be mono-substituted, di-substituted or tri-substituted. In some embodiments, Alk² can be mono-substituted with fluoro or unsubstituted C₁-C₃ alkyl, such as those described herein. In some embodiments, Alk² can be mono-substituted with fluoro or unsubstituted methyl. In some embodiments, Alk² can be di-substituted with one fluoro and one unsubstituted C₁-C₃ alkyl, such as those described herein. In some embodiments, Alk² can be di-substituted with one fluoro and one unsubstituted methyl. In some embodiments, Alk² can be di-substituted with two independently selected unsubstituted C₁-C₃ alkyl, such as those described herein. In some embodiments, Alk² can be di-substituted with unsubstituted methyl.

In some embodiments, Alk² can be selected from:

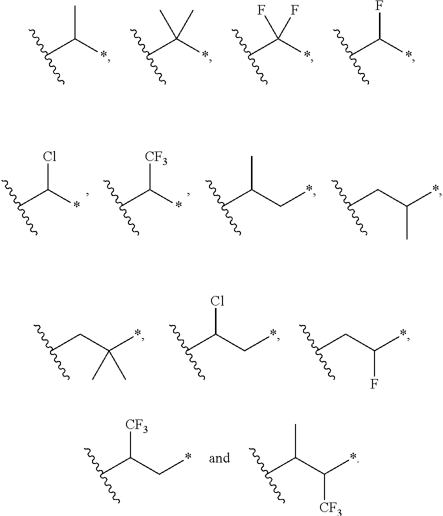

In some embodiments, p can be 0. When p is 0, those skilled in the art understand that the (CHR⁸) group is directly connected to X³. In some embodiments, p can be 1.

In some embodiments, the C₁-C₆ alkyl of the substituted or unsubstituted 3 to 10 membered heterocyclyl(C₁-C₆ alkyl) of R⁸ can be a substituted or unsubstituted C₁-C₆ alkyl such as those described herein. In some embodiments, the 3 to 10 membered heterocyclyl of the substituted or unsubstituted 3 to 10 membered heterocyclyl(C₁-C₆ alkyl) of R⁸ can be monocyclic. In some embodiments, the 3 to 10 membered heterocyclyl of the substituted or unsubstituted 3 to 10 membered heterocyclyl(C₁-C₆ alkyl) can be bicyclic. In other embodiments, the 3 to 10 membered heterocyclyl of the substituted or unsubstituted 3 to 10 membered heterocyclyl(C₁-C₆ alkyl) can be connected to the C₁-C₆ alkyl of the substituted or unsubstituted 3 to 10 membered heterocyclyl(C₁-C₆ alkyl) through a carbon atom. In some embodiments, the 3 to 10 membered heterocyclyl of the substituted or unsubstituted 3 to 10 membered heterocyclyl(C₁-C₆ alkyl) can be unsubstituted. In other embodiments, the 3 to 10 membered heterocyclyl of the substituted or unsubstituted 3 to 10 membered heterocyclyl(C₁-C₆ alkyl) can be substituted. In some embodiments, the 3 to 10 membered heterocyclyl of the substituted or unsubstituted 3 to 10 membered heterocyclyl(C₁-C₆ alkyl) can be substituted on one or more nitrogen atoms. Examples of suitable substituted or unsubstituted monocyclic or bicyclic 3 to 10 membered heterocyclyl groups of R⁸ include, but are not limited to azidirine, oxirane, azetidine, oxetane, pyrrolidine, tetrahydrofuran, imidazoline, pyrazolidine, piperidine, tetrahydropyran, piperazine, morpholine, thiomorpholine, dioxane, 2-azaspiro[3.3]heptane, 2-oxaspiro[3.3]heptane, 2,6-diazaspiro[3.3]heptane, 2-oxa-6-azaspiro[3.3]heptane, 2-azaspiro [3.4]octane, 6-oxaspiro[3.4]octane, 6-oxa-2-azaspiro [3.4]octane, 7-oxa-2-azaspiro[3.5]nonane, 7-oxaspiro[3.5]nonane and 2-oxa-8-azaspiro[4.5]decane. In some embodiments, the C₁-C₆ alkyl of R⁸ can be an unsubstituted methyl or an unsubstituted ethyl and the substituted or unsubstituted 3 to 10 membered heterocyclyl of R⁸ can be a piperidine, tetrahydropyran, piperazine, morpholine, thiomorpholine, dioxane, 2-azaspiro[3.3]heptane, 2-oxaspiro[3.3]heptane, 2,6-diazaspiro[3.3]heptane, 2-oxa-6-azaspiro[3.3]heptane, 2-azaspiro[3.4]octane, 6-oxaspiro[3.4]octane, 6-oxa-2-azaspiro[3.4]octane, 7-oxa-2-azaspiro[3.5]nonane, 7-oxaspiro[3.5]nonane or 2-oxa-8-azaspiro[4.5]decane.

In some embodiments, R⁸ can be a substituted or unsubstituted 6 to 10 membered spiro heterocyclyl(C₁-C₆ alkyl). In some embodiments, the C₁-C₆ alkyl of the substituted or unsubstituted 6 to 10 membered spiro heterocyclyl(C₁-C₆ alkyl) of R⁸ can be a substituted or unsubstituted C₁-C₆ alkyl, such as those described herein. In some embodiments, the C₁-C₆ alkyl of the substituted or unsubstituted 6 to 10 membered spiro heterocyclyl(C₁-C₆ alkyl) can be unsubstituted. In some embodiments, the 6 to 10 membered spiro heterocyclyl of the substituted or unsubstituted 6 to 10 membered spiro heterocyclyl(C₁-C₆ alkyl) can be connected to the C₁-C₆ alkyl of R⁸ through a nitrogen atom. In other embodiments, the 6 to 10 membered spiro heterocyclyl of the substituted or unsubstituted 6 to 10 membered spiro heterocyclyl(C₁-C₆ alkyl) can be connected to the C₁-C₆ alkyl of the substituted or unsubstituted 6 to 10 membered spiro heterocyclyl(C₁-C₆ alkyl) through a carbon atom. In some embodiments, the substituted or unsubstituted 6 to 10 membered spiro heterocyclyl of the substituted or unsubstituted 6 to 10 membered spiro heterocyclyl(C₁-C₆ alkyl) can be unsubstituted. In other embodiments, 6 to 10 membered spiro heterocyclyl of the substituted or unsubstituted 6 to 10 membered spiro heterocyclyl(C₁-C₆ alkyl) can be substituted. In some embodiments, the 6 to 10 membered spiro heterocyclyl of the substituted or unsubstituted 6 to 10 membered spiro heterocyclyl(C₁-C₆ alkyl) can be substituted on one or more nitrogen atoms. In some embodiments, the substituted or unsubstituted 6 to 10 membered spiro heterocyclyl of the substituted or unsubstituted 6 to 10 membered spiro heterocyclyl(C₁-C₆ alkyl) can be an azaspirohexane, azaspiroheptane, azaspirooctane, oxaspirohexane, oxaspiroheptane, oxaspirooctane, diazaspirohexane, diazaspiroheptane, diazaspirooctane, dioxaspirohexane, dioxaspiroheptane, dioxaspirooctane, oxa-azaspirohexane, oxa-azaspiroheptane or oxa-azaspirooctane. Examples of suitable substituted or unsubstituted 6 to 10 membered spiro heterocyclyl of R⁸ include, but are not limited to, 2-azaspiro[3.3]heptane, 2-oxaspiro[3.3]heptane, 2,6-diazaspiro[3.3]heptane, 2-oxa-6-azaspiro[3.3]heptane, 2-azaspiro[3.4]octane, 6-oxaspiro[3.4]octane, 6-oxa-2-azaspiro[3.4]octane, 7-oxa-2-azaspiro[3.5]nonane, 7-oxaspiro[3.5]nonane and 2-oxa-8-azaspiro[4.5]decane. In some embodiments, the C₁-C₆ alkyl of the substituted or unsubstituted 6 to 10 membered spiro heterocyclyl(C₁-C₆ alkyl) can be an unsubstituted methyl or an unsubstituted ethyl and the 6 to 10 membered spiro heterocyclyl of R⁸ can be an azaspirohexane, azaspiroheptane, azaspirooctane, oxaspirohexane, oxaspiroheptane, oxaspirooctane, diazaspirohexane, diazaspiroheptane, diazaspirooctane, dioxaspirohexane, dioxaspiroheptane, dioxaspirooctane, oxa-azaspirohexane, oxa-azaspiroheptane or oxa-azaspirooctane, for example, 2-azaspiro[3.3]heptane, 2-oxaspiro[3.3]heptane, 2,6-diazaspiro[3.3]heptane, 2-oxa-6-azaspiro[3.3]heptane, 2-azaspiro[3.4]octane, 6-oxaspiro[3.4]octane, 6-oxa-2-azaspiro[3.4]octane, 7-oxa-2-azaspiro[3.5]nonane, 7-oxaspiro[3.5]nonane or 2-oxa-8-azaspiro[4.5]decane.

In some embodiments, R⁸ can be a substituted or unsubstituted di-C₁-C₆ alkylamine(C₁-C₆ alkyl), for example, a di-C₁-C₆ alkylamine(ethyl), di-C₁-C₆ alkylamine(propyl), di-C₁-C₆ alkylamine(butyl), di-C₁-C₆ alkylamine(pentyl) or di-C₁-C₆ alkylamine(hexyl). In some embodiments, each C₁-C₆ alkyl group in the di-C₁-C₆ alkylamine can be the same. In other embodiments, each C₁-C₆ alkyl group in the di-C₁-C₆ alkylamine can be different. Suitable substituted or unsubstituted di-C₁-C₆ alkylamine(C₁-C₆ alkyl) include, but are not limited to, —N(methyl)₂, —N(ethyl)₂, —N(n-propyl)₂, —N(isopropyl)₂, —N(t-butyl)₂, —N(ethyl)(methyl), —N(isopropyl)(methyl), —N(t-butyl)(methyl) and —N(isopropyl)(ethyl); each connected to a substituted or unsubstituted C₁-C₆ alkyl group.

In some embodiments, R⁸ can be a substituted or unsubstituted di-methylamine(C₁-C₆ alkyl), for example,

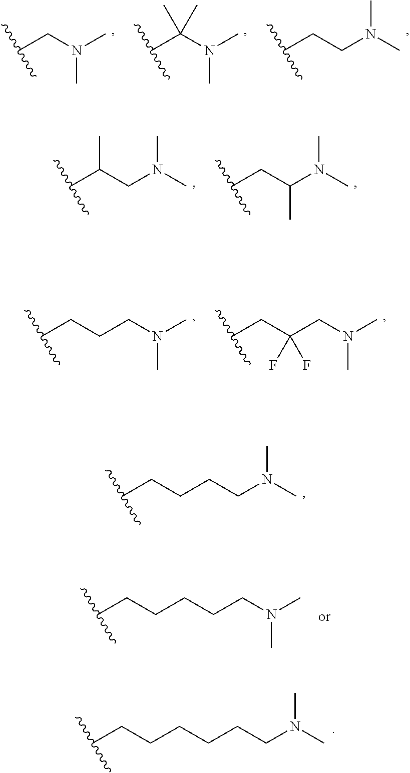

In some embodiments, R⁸ can be a substituted or unsubstituted mono-C₁-C₆ alkylamine(C₁-C₆ alkyl), for example, a substituted or unsubstituted mono-C₁-C₆ alkylamine(ethyl), mono-C₁-C₆ alkylamine(propyl), mono-C₁-C₆ alkylamine(butyl), mono-C₁-C₆ alkylamine(pentyl) or mono-C₁-C₆ alkylamine(hexyl). In some embodiments, the C₁-C₆alkyl of the unsubstituted mono-C₁-C₆ alkylamine(C₁-C₆ alkyl) group can be an unsubstituted C₁-C₆ alkyl, such as those described herein.

In some embodiments, R⁸ can be unsubstituted. In other embodiments, R⁸ can be substituted. In some embodiments, R⁸ can be substituted with 1 or 2 substituents independently selected from an unsubstituted C₁-C₆ alkyl (such as those described herein), an unsubstituted C₁-C₆ alkoxy (such as those described herein), an unsubstituted di-C₁-C₆ alkylamine (such as those described herein), an unsubstituted acyl(C₁-C₆ alkyl) (for example, acetyl or benzoyl), an unsubstituted C-carboxy (for example, —CO₂H, —CO₂—C₁-C₆ alkyl, —CO₂—C₃-C₆ cycloalkyl or —CO₂—C₆-C₁₀ aryl), fluoro, chloro and hydroxy. For example, the 3 to 10 membered heterocyclyl(C₁-C₆ alkyl), di-C₁-C₆ alkylamine(C₁-C₆ alkyl) and mono-C₁-C₆ alkylamine(C₁-C₆ alkyl) groups of R⁸ can be substituted with 1 or 2 substituents independently selected from any of the aforementioned substituents.

In some embodiments, R⁸ can be:

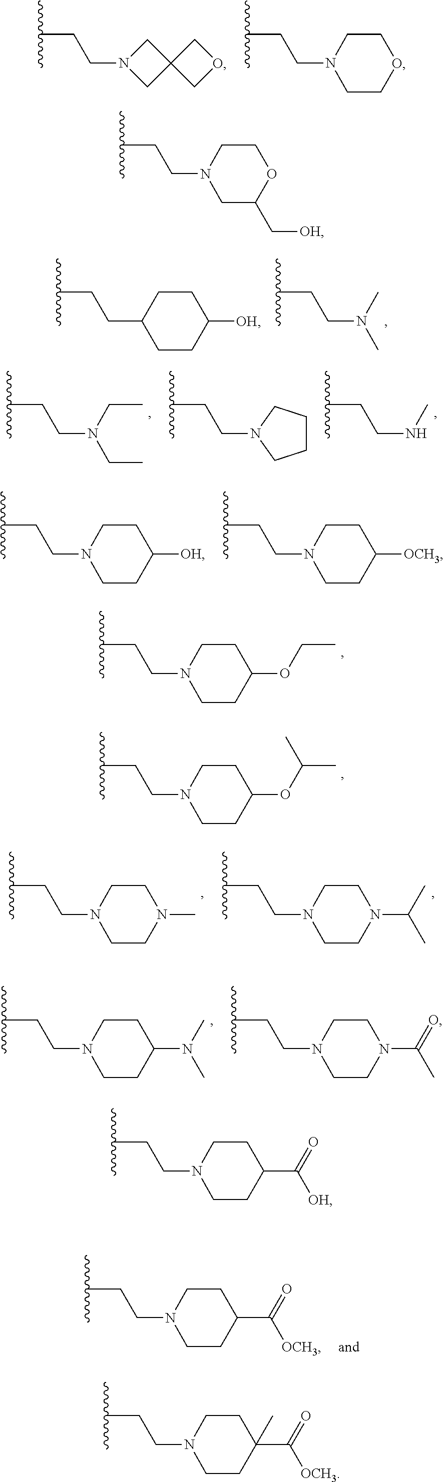

In some embodiments, R⁸ can be

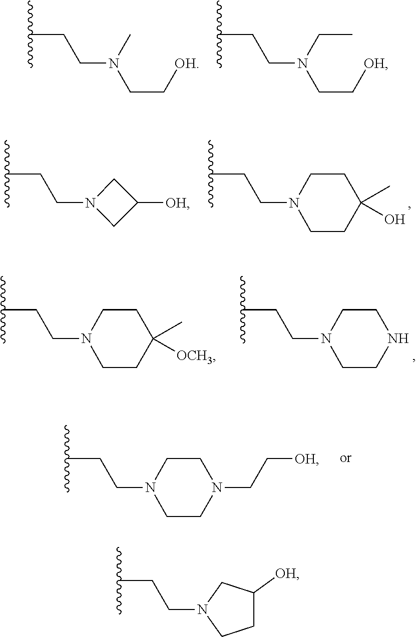

In some embodiments, R⁹ can be a substituted or unsubstituted monocyclic or bicyclic C₆-C₁₀ aryl. In some embodiments, R⁹ can be a substituted monocyclic or bicyclic C₆-C₁₀ aryl. In other embodiments, R⁹ can be an unsubstituted monocyclic or bicyclic C₆-C₁₀ aryl. In some embodiments, R⁹ can be a substituted phenyl or a substituted naphthyl. In some embodiments, R⁹ can be an unsubstituted phenyl or an unsubstituted naphthyl.

In some embodiments, R⁹ can be a substituted or unsubstituted 5 to 10 membered heteroaryl. In some embodiments, R⁹ can be a substituted 5 to 10 membered heteroaryl. In other embodiments, R⁹ can be an unsubstituted 5 to 10 membered heteroaryl. In some embodiments, R⁹ can be a monocyclic substituted or unsubstituted 5 to 10 membered heteroaryl. In other embodiments, R⁹ can be a bicyclic substituted or unsubstituted 5 to 10 membered heteroaryl. Suitable substituted or unsubstituted monocyclic or bicyclic 5 to 10 membered heteroaryl include, but are not limited to, pyrrole, furan, thiophene, imidazole, pyrazole, oxazole, isoxazole, thiazole, isothiazole, triazole, pyridine, pyridazine, pyrimidine, pyrazine, pyrrolo-pyrroles, pyrrolo-furans, pyrrolo-thiophenes, indole, isoindole, indolizine, indazole, benzimidazole, azaindoles, purine, benzofuran, isobenzofuran, benzothiophene, isobenzothiophene, quinoline, isoquinoline, quinoxaline, phthalazine, quinazoline, cinnoline, 1,8-naphthyridine, pyrido-pyrimidines and pteridine.

In some embodiments, R³ is hydrogen or halogen. For example, an embodiment provides a pharmaceutical composition comprising a compound of Formula (I), or a pharmaceutically acceptable salt thereof, and a pharmaceutically acceptable carrier comprising albumin, wherein:

R¹ is selected from the group consisting of hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamine and an unsubstituted di-C₁-C₆ alkylamine;

each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl; or

when m is 2 or 3, each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl, or two R² groups taken together with the atom(s) to which they are attached form a substituted or unsubstituted C₃-C₆ cycloalkyl or a substituted or unsubstituted 3 to 6 membered heterocyclyl;

R³ is hydrogen or halogen;

R⁴ is selected from the group consisting of NO₂, S(O)R⁶, SO₂R⁶, halogen, cyano and an unsubstituted C₁-C₆ haloalkyl;

R⁵ is selected from the group consisting of —X¹-(Alk¹)ₙ-R⁷ and —X²(CHR⁸)-(Alk²)ₚ-X³-R⁹;

Alk¹ and Alk² are independently selected from an unsubstituted C₁-C₄ alkylene and a C₁-C₄ alkylene substituted with 1, 2 or 3 substituents independently selected from fluoro, chloro, an unsubstituted C₁-C₃ alkyl and an unsubstituted C₁-C₃ haloalkyl;

R⁶ is selected from the group consisting of a substituted or unsubstituted C₁-C₆ alkyl, a substituted or unsubstituted C₁-C₆ haloalkyl and a substituted or unsubstituted C₃-C₆ cycloalkyl;

R⁷ is selected from a substituted or unsubstituted C₁-C₆ alkoxy, a substituted or unsubstituted C₃-C₁₀ cycloalkyl, a substituted or unsubstituted 3 to 10 membered heterocyclyl, hydroxy, amino, a substituted or unsubstituted mono-substituted amine group, a substituted or unsubstituted di-substituted amine group, a substituted or unsubstituted N-carbamyl, a substituted or unsubstituted C-amido and a substituted or unsubstituted N-amido;

R⁸ is selected from a substituted or unsubstituted 3 to 10 membered heterocyclyl(C₁-C₆ alkyl), a substituted or unsubstituted di-C₁-C₆ alkylamine(C₁-C₆ alkyl) and a substituted or unsubstituted mono-C₁-C₆ alkylamine(C₁-C₆ alkyl);

R⁹ is selected from a substituted or unsubstituted 5 to 10 membered heteroaryl and a substituted or unsubstituted C₆-C₁₀ aryl;

m is 0, 1, 2 or 3;

n and p are independently selected from 0 and 1; and

X¹, X² and X³ are independently selected from the group consisting of —O—, —S— and —NH—.

In some embodiments, R³ is selected from the group consisting of X—R^{3A},

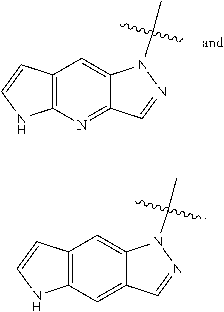 [SMILES: *n1ncc2nc3[nH]ccc3cc21 ; *n1ncc2cc3[nH]ccc3cc21]

For example, an embodiment provides a pharmaceutical composition comprising a compound of Formula (I), or a pharmaceutically acceptable salt thereof, and a pharmaceutically acceptable carrier comprising albumin, wherein:

R¹ is selected from the group consisting of hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamine and an unsubstituted di-C₁-C₆ alkylamine;

each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl; or

when m is 2 or 3, each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl, or two R² groups taken together with the atom(s) to which they are attached form a substituted or unsubstituted C₃-C₆ cycloalkyl or a substituted or unsubstituted 3 to 6 membered heterocyclyl;

R³ is selected from the group consisting of X—R^{3A},

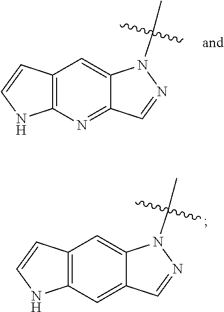 [SMILES: *n1ncc2nc3[nH]ccc3cc21 ; *n1ncc2cc3[nH]ccc3cc21]

R^{3A} is a substituted or unsubstituted 5 to 10 membered heteroaryl;

R⁴ is selected from the group consisting of NO₂, S(O)R⁶, SO₂R⁶, halogen, cyano and an unsubstituted C₁-C₆ haloalkyl;

R⁵ is selected from the group consisting of —X¹-(Alk¹)ₙ-R⁷ and —X²(CHR⁸)-(Alk²)ₚ-X³-R⁹;

Alk¹ and Alk² are independently selected from an unsubstituted C₁-C₄ alkylene and a C₁-C₄ alkylene substituted with 1, 2 or 3 substituents independently selected from fluoro, chloro, an unsubstituted C₁-C₃ alkyl and an unsubstituted C₁-C₃ haloalkyl;

R⁶ is selected from the group consisting of a substituted or unsubstituted C₁-C₆ alkyl, a substituted or unsubstituted C₁-C₆ haloalkyl and a substituted or unsubstituted C₃-C₆ cycloalkyl;

R⁷ is selected from a substituted or unsubstituted C₁-C₆ alkoxy, a substituted or unsubstituted C₃-C₁₀ cycloalkyl, a substituted or unsubstituted 3 to 10 membered heterocyclyl, hydroxy, amino, a substituted or unsubstituted mono-substituted amine group, a substituted or unsubstituted di-substituted amine group, a substituted or unsubstituted N-carbamyl, a substituted or unsubstituted C-amido and a substituted or unsubstituted N-amido;

R⁸ is selected from a substituted or unsubstituted 3 to 10 membered heterocyclyl(C₁-C₆ alkyl), a substituted or unsubstituted di-C₁-C₆ alkylamine(C₁-C₆ alkyl) and a substituted or unsubstituted mono-C₁-C₆ alkylamine(C₁-C₆ alkyl);

R⁹ is selected from a substituted or unsubstituted 5 to 10 membered heteroaryl and a substituted or unsubstituted C₆-C₁₀ aryl;

m is 0, 1, 2 or 3;

n and p are independently selected from 0 and 1;

X, X¹, X² and X³ are independently selected from the group consisting of —O—, —S— and —NH—.

In some embodiments, the compound of Formula (I), or a pharmaceutically acceptable salt thereof, can be selected from a compound of Formula (Ia), Formula (Ib), Formula (Ic) Formula (Id), Formula (Ie), or Formula (If):

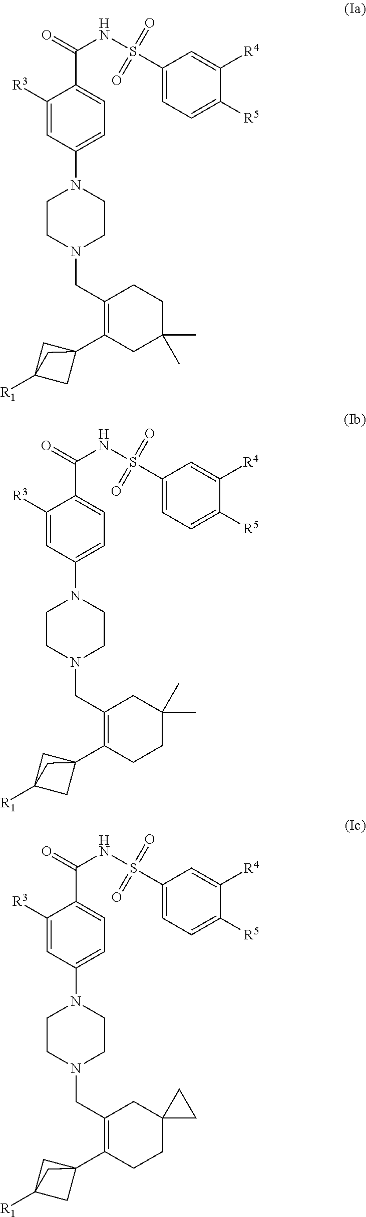

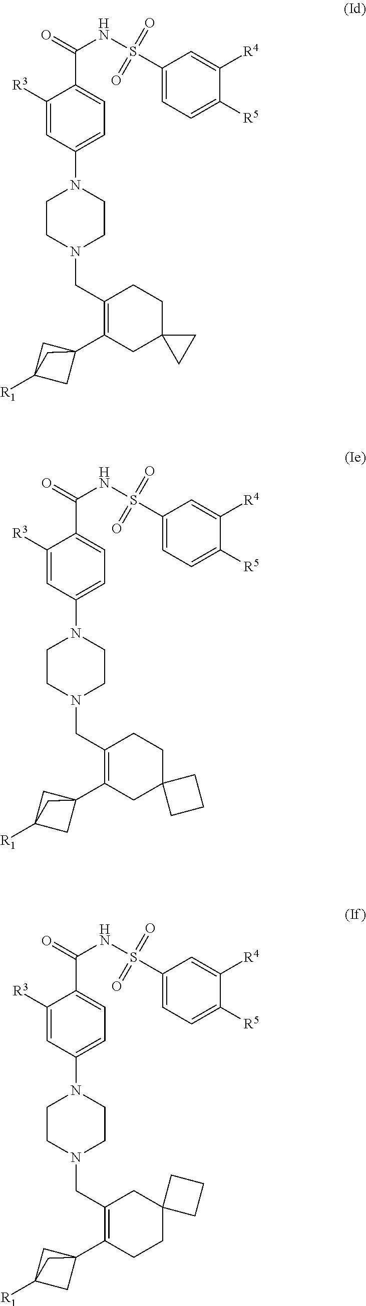

or pharmaceutically acceptable salts of any of the foregoing.

In some embodiments of Formulae (Ia), (Ib), (Ic), (Id), (Ie) and/or (If), R³ can be hydrogen,

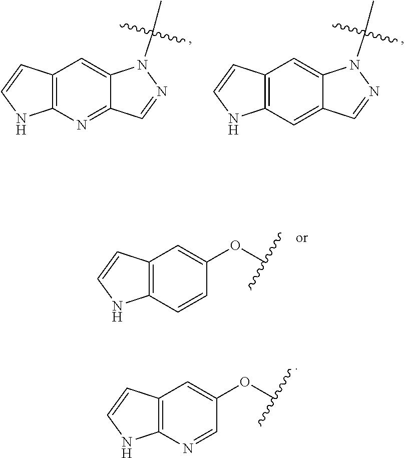 [SMILES: *n1ncc2nc3[nH]ccc3cc21 ; *n1ncc2cc3[nH]ccc3cc21 ; *Oc1ccc2[nH]ccc2c1 ; *Oc1cnc2[nH]ccc2c1]

In some embodiments of Formulae (Ia), (Ib), (Ic), (Id), (Ie) and/or (If), R⁴ can be nitro or —SO₂CF₃. In some embodiments of Formulae (Ia), (Ib), (Ic), (Id), (Ie) and/or (If), R¹ can be fluoro, chloro, —CH₃, —CH₂CH₃, —CHF₂, —CF₃, —CH₂CF₃, —CF₂CH₃, —CF₂CF₃—OCH₃, —OCH₂CH₃, —NHCH₃, —NHCH₂CH₃, —N(CH₃)₂ or —N(CH₂CH₃)₂. In some embodiments of Formulae (Ia), (Ib), (Ic), (Id), (Ie) and/or (If), R⁵ can be —O—R⁷ or —NH—R⁷. In some embodiments Formulae (Ia), (Ib), (Ic), (Id), (Ie) and/or (If), R⁵ can be —O-Alk¹-R⁷ or —NH-Alk¹-R⁷. In some embodiments of Formulae (Ia), (Ib), (Ic), (Id), (Ie) and/or (If), Alk¹ can be an unsubstituted methylene, an unsubstituted ethylene, or an ethylene mono-substituted with —CH₃. In some embodiments of Formulae (Ia), (Ib), (Ic), (Id), (Ie) and/or (If), R⁷ can be an unsubstituted cyclohexanyl or a cyclohexanyl substituted with one or two substituents independently selected from hydroxy, amino, fluoro and unsubstituted C₁-C₃ alkyl (such as those described herein). In some embodiments of this paragraph, R⁷ can be a substituted or unsubstituted monocyclic 5 or 6 membered heterocyclyl, for example, pyrrolidine, piperidine, morpholine, piperazine or tetrahydropyran; wherein each of the aforementioned substituted groups can be substituted with 1 or 2 substituents independently selected from hydroxy, amino, fluoro, an unsubstituted C₁-C₃ alkyl (such as those described herein), an unsubstituted C₁-C₃ alkoxy (such as those described herein), or —SO₂CH₃. In some embodiments of Formulae (Ia), (Ib), (Ic), (Id), (Ie) and/or (If), R⁷ can be connected to Alk¹ by a nitrogen atom. In some embodiments of Formulae (Ia), (Ib), (Ic), (Id), (Ie) and/or (If), R⁷ can be connected to Alk¹ by a carbon atom. In some embodiments of Formulae (Ia), (Ib), (Ic), (Id), (Ie) and/or (If), R⁷ can be substituted on one or more nitrogen atoms. In some embodiments of Formulae (Ia), (Ib), (Ic), (Id), (Ie) and/or (If), R⁵ can be —NH—(CHR⁸-Alk²-S—R⁹, —O—(CHR⁸-Alk²-S—R⁹, —NH—(CHR⁸-Alk²-O—R⁹ or —O—(CHR⁸-Alk²-O—R⁹. In some embodiments of Formulae (Ia), (Ib), (Ic), (Id), (Ie) and/or (If), Alk² can be an unsubstituted methylene, an unsubstituted ethylene, a methylene mono-substituted with —CH₃ or a methylene di-substituted with —CH₃. In some embodiments of Formulae ((Ia), (Ib), (Ic), (Id), (Ie) and/or (If), R⁸ can be an unsubstituted di-C₁-C₃ alkylamine(methyl) or an unsubstituted di-C₁-C₃ alkylamine(ethyl). In some embodiments of Formulae (Ia), (Ib), (Ic), (Id), (Ie) and/or (If), R⁸ can be a substituted or unsubstituted 5 to 7 membered heterocyclyl(C₁-C₆ alkyl); wherein the C₁-C₆ alkyl can be an unsubstituted methyl, an unsubstituted ethyl or an unsubstituted n-propyl; the 5 to 7 membered heterocyclyl can (a) be monocyclic or spiro, (b) include 1 oxygen atom, 1 nitrogen atom, or 1 oxygen atom and one nitrogen atom, (c) be unsubstituted or substituted with 1 or 2 substituents independently selected from an unsubstituted C₁-C₃ alkyl (such as those described herein), —N(CH₃)₂, —N(CH₂CH₃)₂, an unsubstituted acetyl, —CO₂H, fluoro or hydroxy. In some embodiments of Formulae (Ia), (Ib), (Ic), (Id), (le) and/or (If), R⁹ can be unsubstituted phenyl.

Examples of a compound of Formula (I) include:

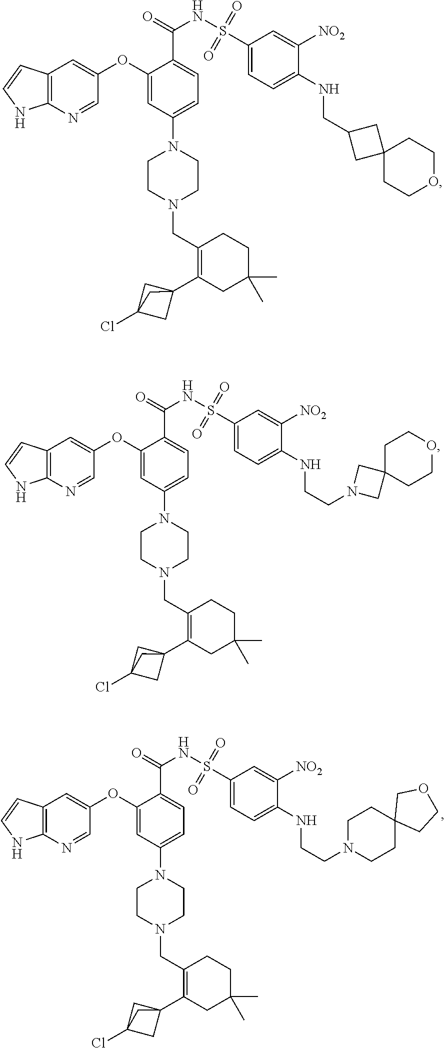 [SMILES: CC1(C)CCC(CN2CCN(c3ccc(C(=O)NS(=O)(=O)c4ccc(NCC5CC6(CCOCC6)C5)c([N+](=O)[O-])c4)c(Oc4cnc5[nH]ccc5c4)c3)CC2)=C(C23CC(Cl)(C2)C3)C1 ; CC1(C)CCC(CN2CCN(c3ccc(C(=O)NS(=O)(=O)c4ccc(NCCN5CC6(CCOCC6)C5)c([N+](=O)[O-])c4)c(Oc4cnc5[nH]ccc5c4)c3)CC2)=C(C23CC(Cl)(C2)C3)C1 ; CC1(C)CCC(CN2CCN(c3ccc(C(=O)NS(=O)(=O)c4ccc(NCCN5CCC6(CCOC6)CC5)c([N+](=O)[O-])c4)c(Oc4cnc5[nH]ccc5c4)c3)CC2)=C(C23CC(Cl)(C2)C3)C1]

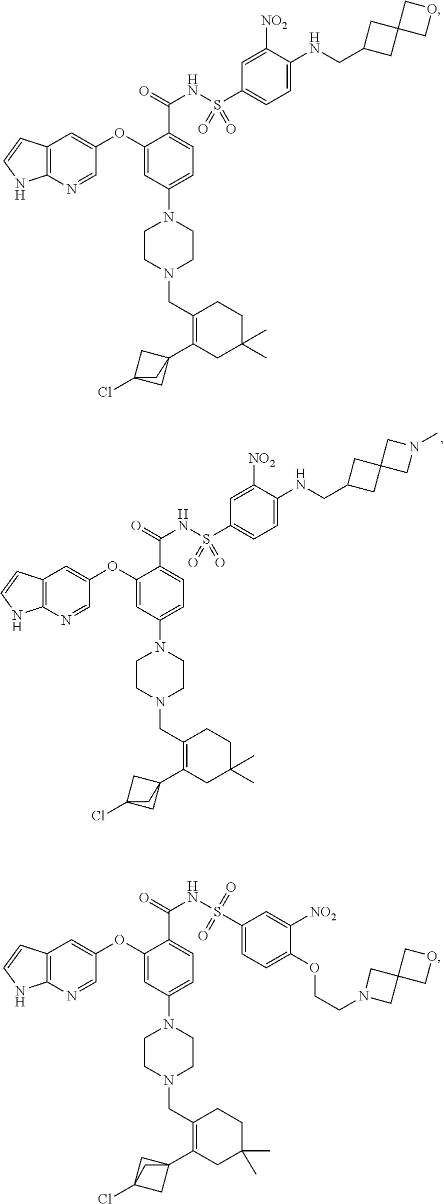 [SMILES: CC1(C)CCC(CN2CCN(c3ccc(C(=O)NS(=O)(=O)c4ccc(NCC5CC6(COC6)C5)c([N+](=O)[O-])c4)c(Oc4cnc5[nH]ccc5c4)c3)CC2)=C(C23CC(Cl)(C2)C3)C1 ; CN1CC2(CC(CNc3ccc(S(=O)(=O)NC(=O)c4ccc(N5CCN(CC6=C(C78CC(Cl)(C7)C8)CC(C)(C)CC6)CC5)cc4Oc4cnc5[nH]ccc5c4)cc3[N+](=O)[O-])C2)C1 ; CC1(C)CCC(CN2CCN(c3ccc(C(=O)NS(=O)(=O)c4ccc(OCCN5CC6(COC6)C5)c([N+](=O)[O-])c4)c(Oc4cnc5[nH]ccc5c4)c3)CC2)=C(C23CC(Cl)(C2)C3)C1]

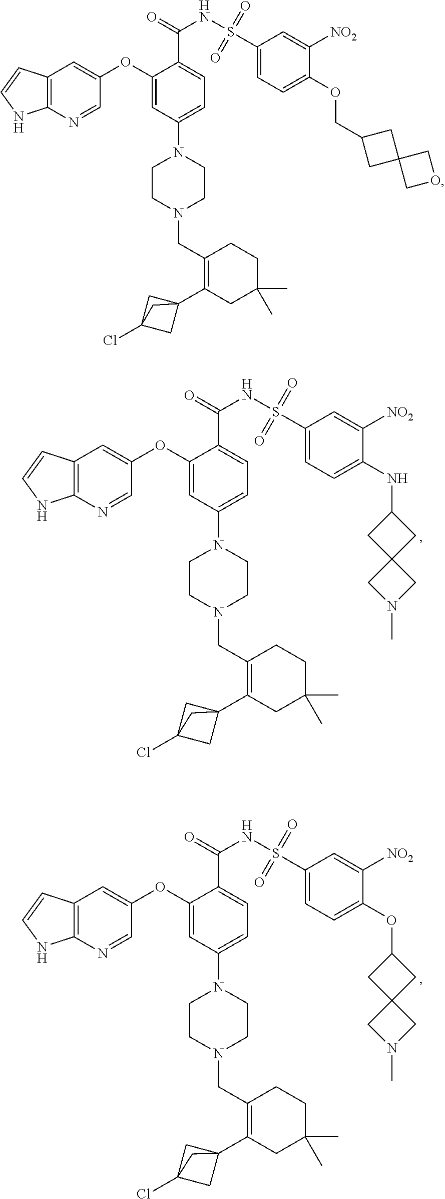 [SMILES: CC1(C)CCC(CN2CCN(c3ccc(C(=O)NS(=O)(=O)c4ccc(OCC5CC6(COC6)C5)c([N+](=O)[O-])c4)c(Oc4cnc5[nH]ccc5c4)c3)CC2)=C(C23CC(Cl)(C2)C3)C1 ; CN1CC2(CC(Nc3ccc(S(=O)(=O)NC(=O)c4ccc(N5CCN(CC6=C(C78CC(Cl)(C7)C8)CC(C)(C)CC6)CC5)cc4Oc4cnc5[nH]ccc5c4)cc3[N+](=O)[O-])C2)C1 ; CN1CC2(CC(Oc3ccc(S(=O)(=O)NC(=O)c4ccc(N5CCN(CC6=C(C78CC(Cl)(C7)C8)CC(C)(C)CC6)CC5)cc4Oc4cnc5[nH]ccc5c4)cc3[N+](=O)[O-])C2)C1]

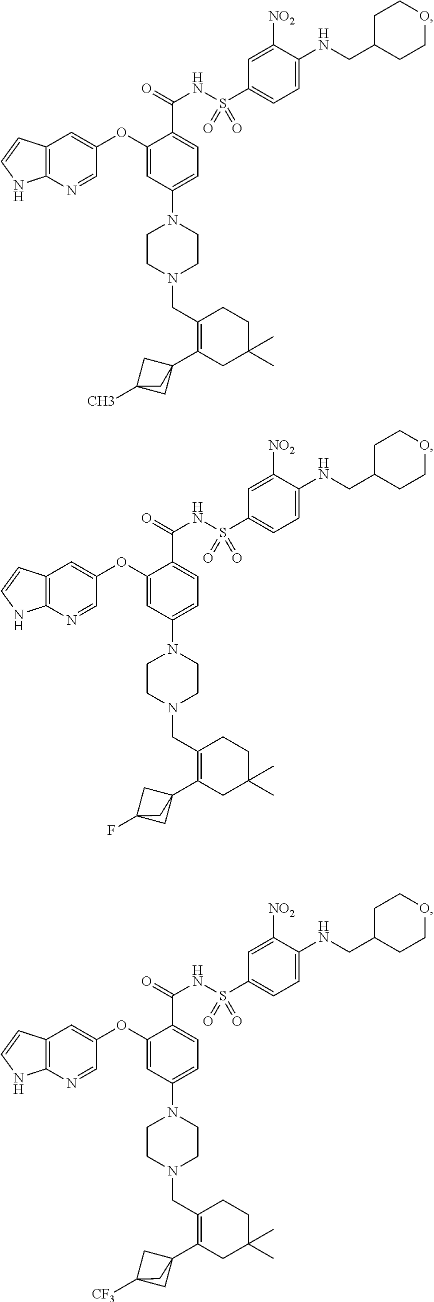 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(C23CC(C)(C2)C3)C1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(C23CC(F)(C2)C3)C1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(C23CC(C(F)(F)F)(C2)C3)C1]

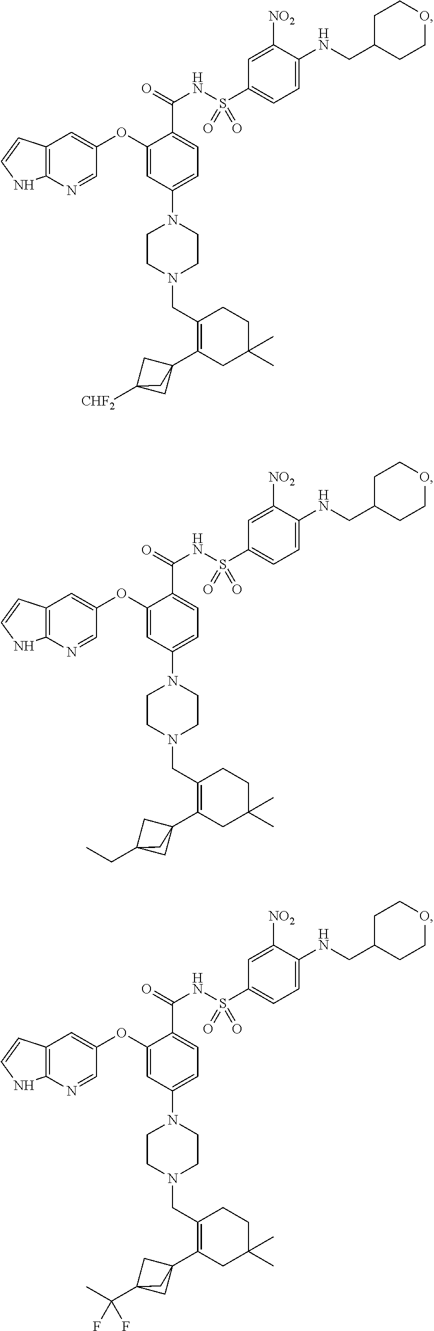 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(C23CC(C(F)F)(C2)C3)C1 ; CCC12CC(C3=C(CN4CCN(c5ccc(C(=O)NS(=O)(=O)c6ccc(NCC7CCOCC7)c([N+](=O)[O-])c6)c(Oc6cnc7[nH]ccc7c6)c5)CC4)CCC(C)(C)C3)(C1)C2 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(C23CC(C(C)(F)F)(C2)C3)C1]

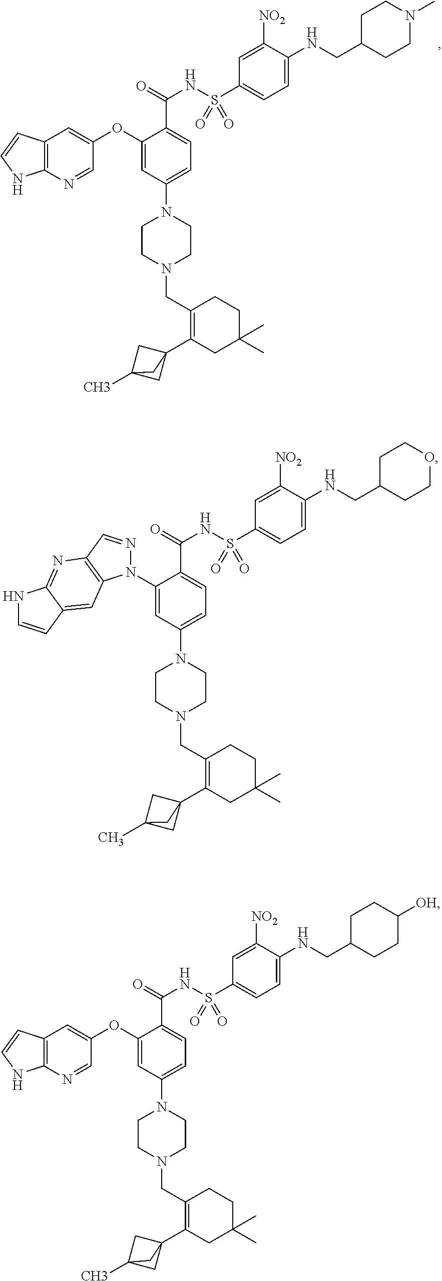 [SMILES: CN1CCC(CNc2ccc(S(=O)(=O)NC(=O)c3ccc(N4CCN(CC5=C(C67CC(C)(C6)C7)CC(C)(C)CC5)CC4)cc3Oc3cnc4[nH]ccc4c3)cc2[N+](=O)[O-])CC1 ; CC1(C)CCC(CN2CCN(c3ccc(C(=O)NS(=O)(=O)c4ccc(NCC5CCOCC5)c([N+](=O)[O-])c4)c(-n4ncc5nc6[nH]ccc6cc54)c3)CC2)=C(C23CC(C)(C2)C3)C1 ; CC1(C)CCC(CN2CCN(c3ccc(C(=O)NS(=O)(=O)c4ccc(NCC5CCC(O)CC5)c([N+](=O)[O-])c4)c(Oc4cnc5[nH]ccc5c4)c3)CC2)=C(C23CC(C)(C2)C3)C1]

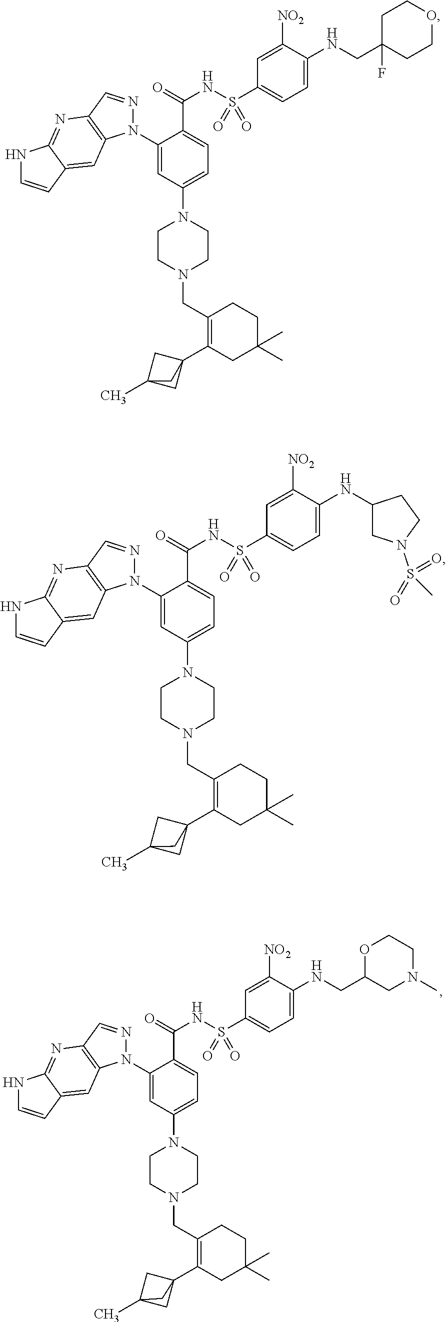 [SMILES: CC1(C)CCC(CN2CCN(c3ccc(C(=O)NS(=O)(=O)c4ccc(NCC5(F)CCOCC5)c([N+](=O)[O-])c4)c(-n4ncc5nc6[nH]ccc6cc54)c3)CC2)=C(C23CC(C)(C2)C3)C1 ; CC1(C)CCC(CN2CCN(c3ccc(C(=O)NS(=O)(=O)c4ccc(NC5CCN(S(C)(=O)=O)C5)c([N+](=O)[O-])c4)c(-n4ncc5nc6[nH]ccc6cc54)c3)CC2)=C(C23CC(C)(C2)C3)C1 ; CN1CCOC(CNc2ccc(S(=O)(=O)NC(=O)c3ccc(N4CCN(CC5=C(C67CC(C)(C6)C7)CC(C)(C)CC5)CC4)cc3-n3ncc4nc5[nH]ccc5cc43)cc2[N+](=O)[O-])C1]

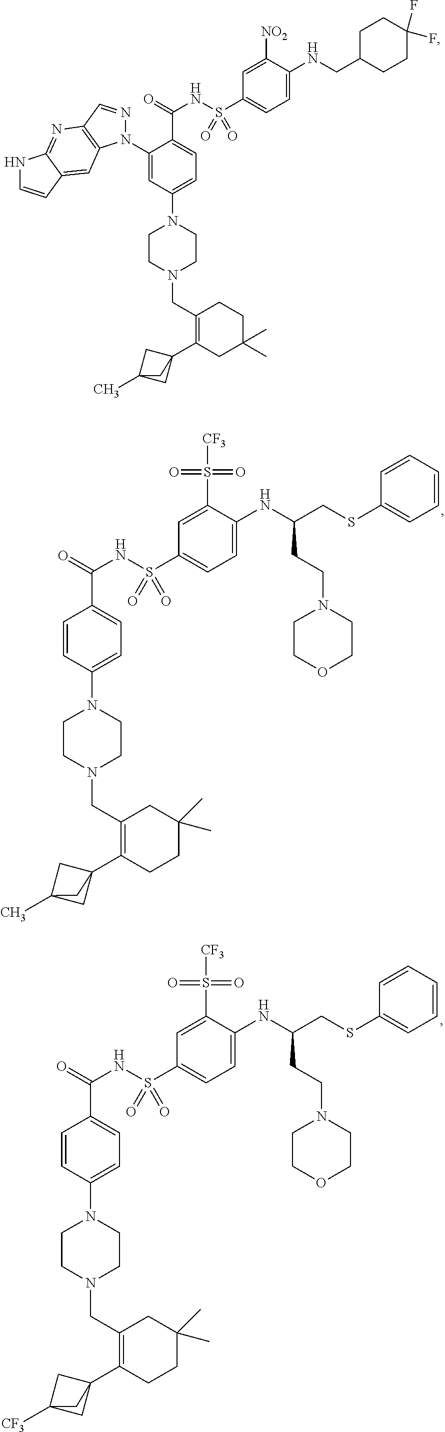 [SMILES: CC1(C)CCC(CN2CCN(c3ccc(C(=O)NS(=O)(=O)c4ccc(NCC5CCC(F)(F)CC5)c([N+](=O)[O-])c4)c(-n4ncc5nc6[nH]ccc6cc54)c3)CC2)=C(C23CC(C)(C2)C3)C1 ; CC1(C)CCC(C23CC(C)(C2)C3)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1 ; CC1(C)CCC(C23CC(C(F)(F)F)(C2)C3)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

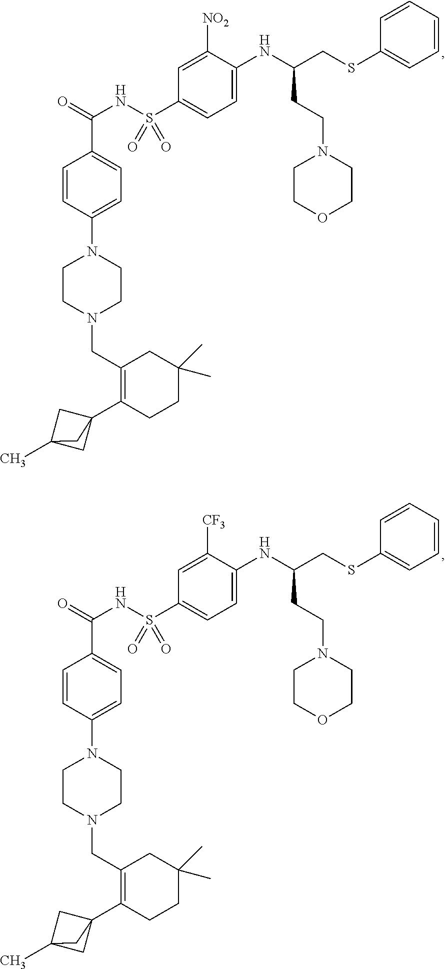 [SMILES: CC1(C)CCC(C23CC(C)(C2)C3)=C(CN2CCN(c3ccc(C(=O)NS(=O)(=O)c4ccc(N[C@H](CCN5CCOCC5)CSc5ccccc5)c([N+](=O)[O-])c4)cc3)CC2)C1 ; CC1(C)CCC(C23CC(C)(C2)C3)=C(CN2CCN(c3ccc(C(=O)NS(=O)(=O)c4ccc(N[C@H](CCN5CCOCC5)CSc5ccccc5)c(C(F)(F)F)c4)cc3)CC2)C1]

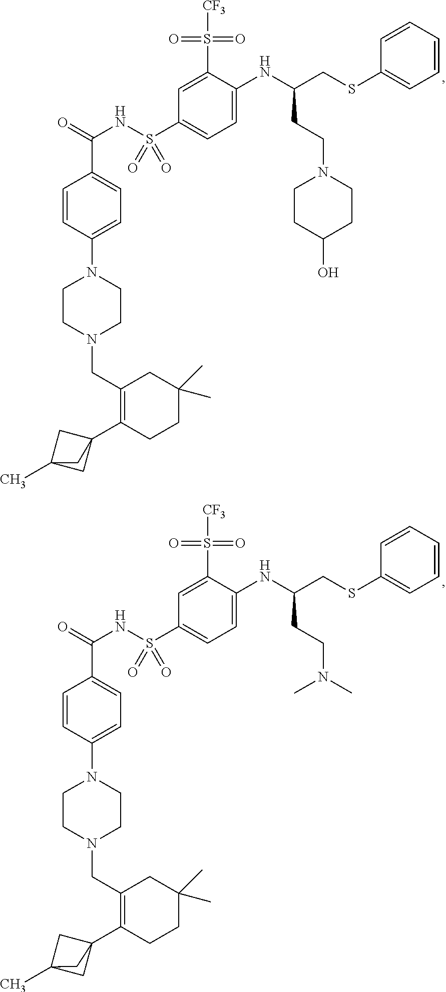 [SMILES: CC1(C)CCC(C23CC(C)(C2)C3)=C(CN2CCN(c3ccc(C(=O)NS(=O)(=O)c4ccc(N[C@H](CCN5CCC(O)CC5)CSc5ccccc5)c(S(=O)(=O)C(F)(F)F)c4)cc3)CC2)C1 ; CN(C)CC[C@H](CSc1ccccc1)Nc1ccc(S(=O)(=O)NC(=O)c2ccc(N3CCN(CC4=C(C56CC(C)(C5)C6)CCC(C)(C)C4)CC3)cc2)cc1S(=O)(=O)C(F)(F)F]

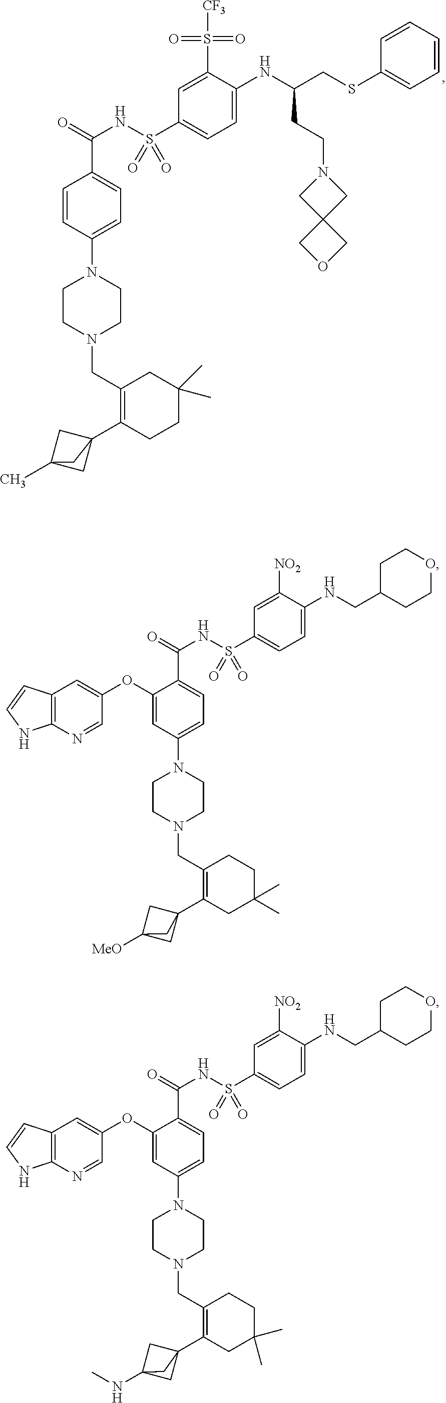 [SMILES: CC1(C)CCC(C23CC(C)(C2)C3)=C(CN2CCN(c3ccc(C(=O)NS(=O)(=O)c4ccc(N[C@H](CCN5CC6(COC6)C5)CSc5ccccc5)c(S(=O)(=O)C(F)(F)F)c4)cc3)CC2)C1 ; COC12CC(C3=C(CN4CCN(c5ccc(C(=O)NS(=O)(=O)c6ccc(NCC7CCOCC7)c([N+](=O)[O-])c6)c(Oc6cnc7[nH]ccc7c6)c5)CC4)CCC(C)(C)C3)(C1)C2 ; CNC12CC(C3=C(CN4CCN(c5ccc(C(=O)NS(=O)(=O)c6ccc(NCC7CCOCC7)c([N+](=O)[O-])c6)c(Oc6cnc7[nH]ccc7c6)c5)CC4)CCC(C)(C)C3)(C1)C2]

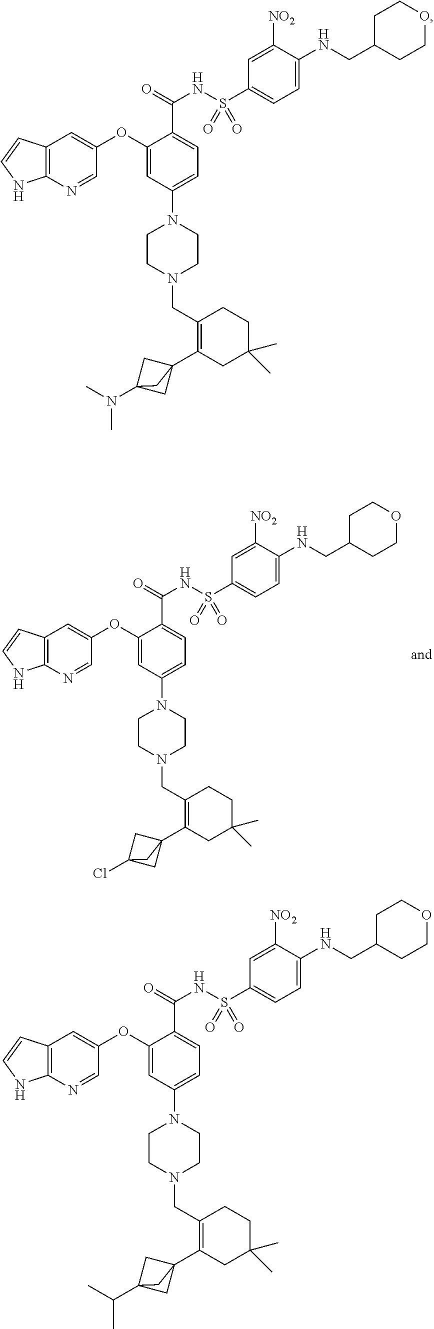 [SMILES: CN(C)C12CC(C3=C(CN4CCN(c5ccc(C(=O)NS(=O)(=O)c6ccc(NCC7CCOCC7)c([N+](=O)[O-])c6)c(Oc6cnc7[nH]ccc7c6)c5)CC4)CCC(C)(C)C3)(C1)C2 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(C23CC(Cl)(C2)C3)C1 ; "and" ; CC(C)C12CC(C3=C(CN4CCN(c5ccc(C(=O)NS(=O)(=O)c6ccc(NCC7CCOCC7)c([N+](=O)[O-])c6)c(Oc6cnc7[nH]ccc7c6)c5)CC4)CCC(C)(C)C3)(C1)C2]

or a pharmaceutically acceptable salt of any of the foregoing.

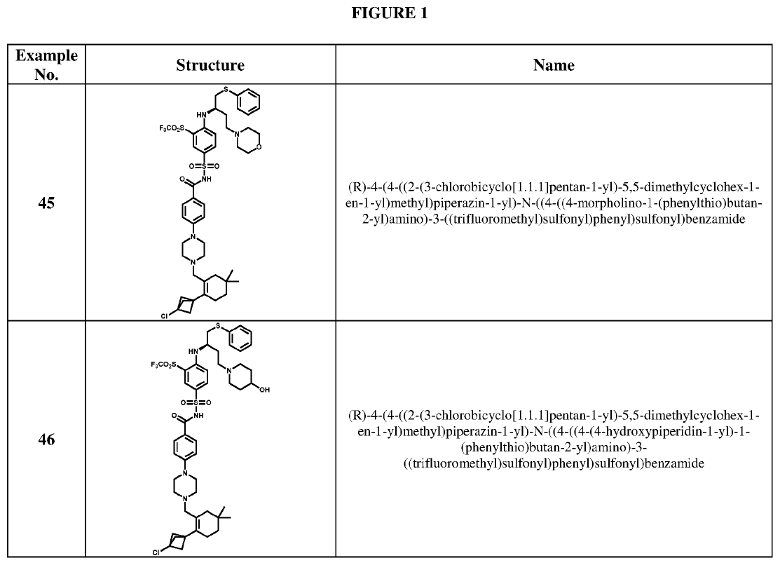

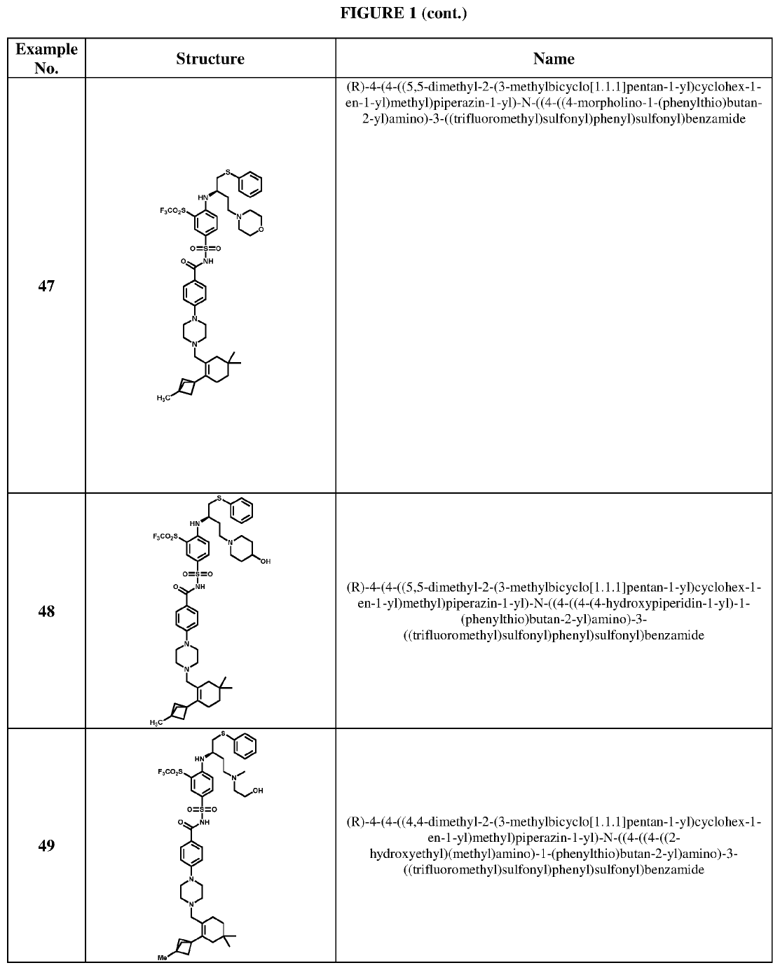

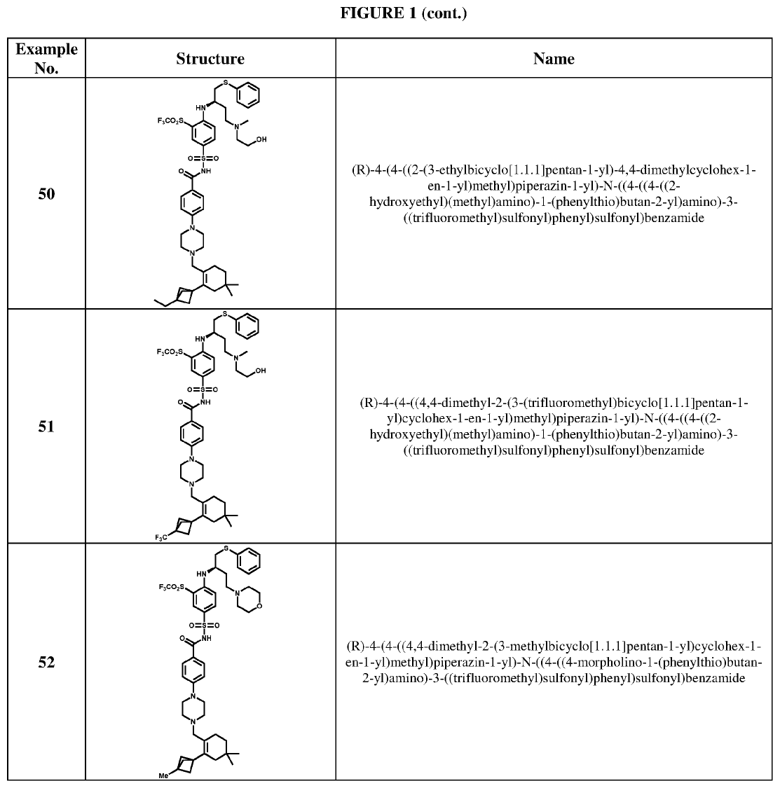

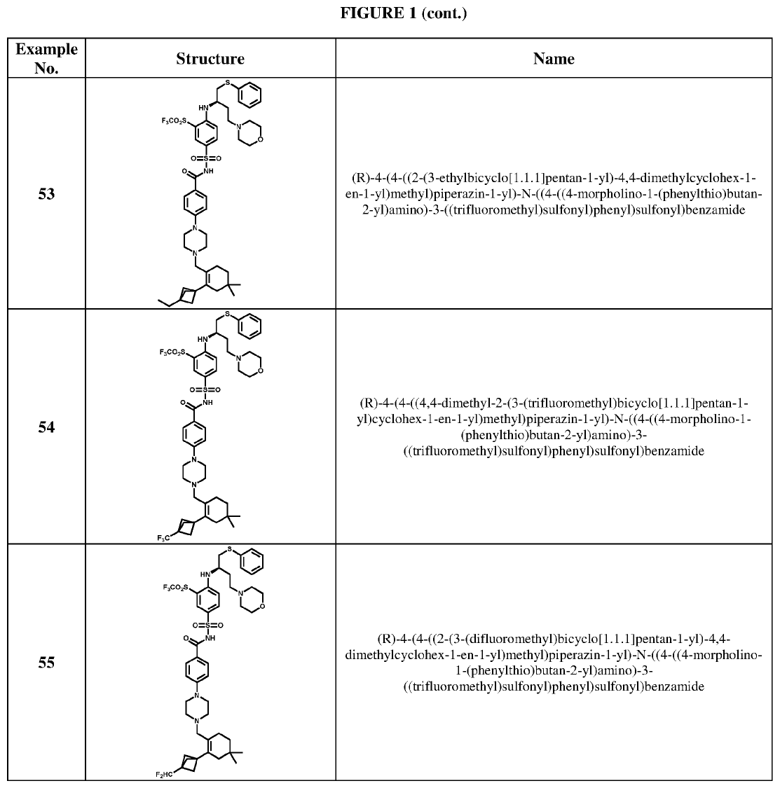

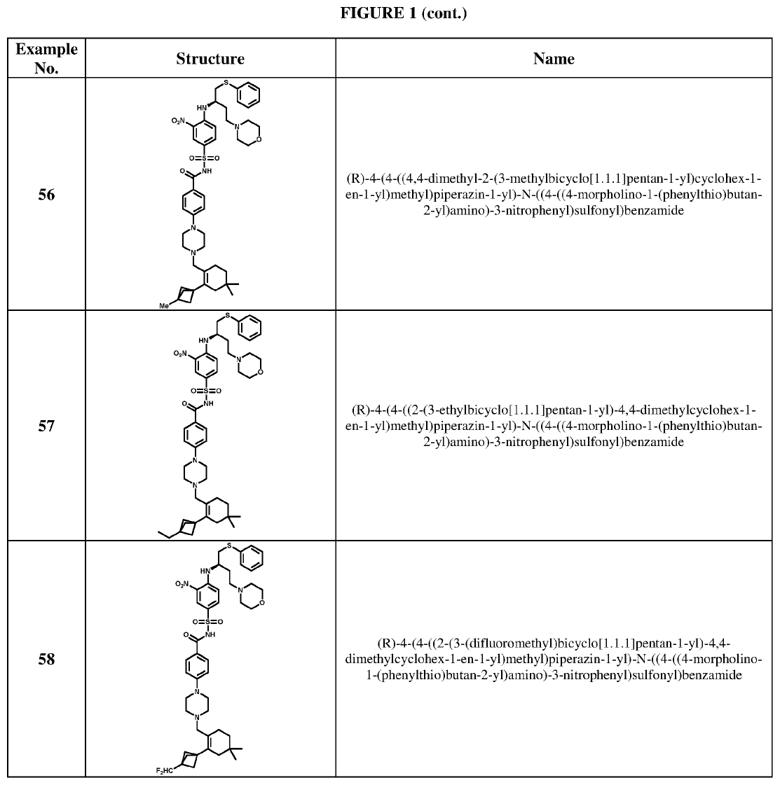

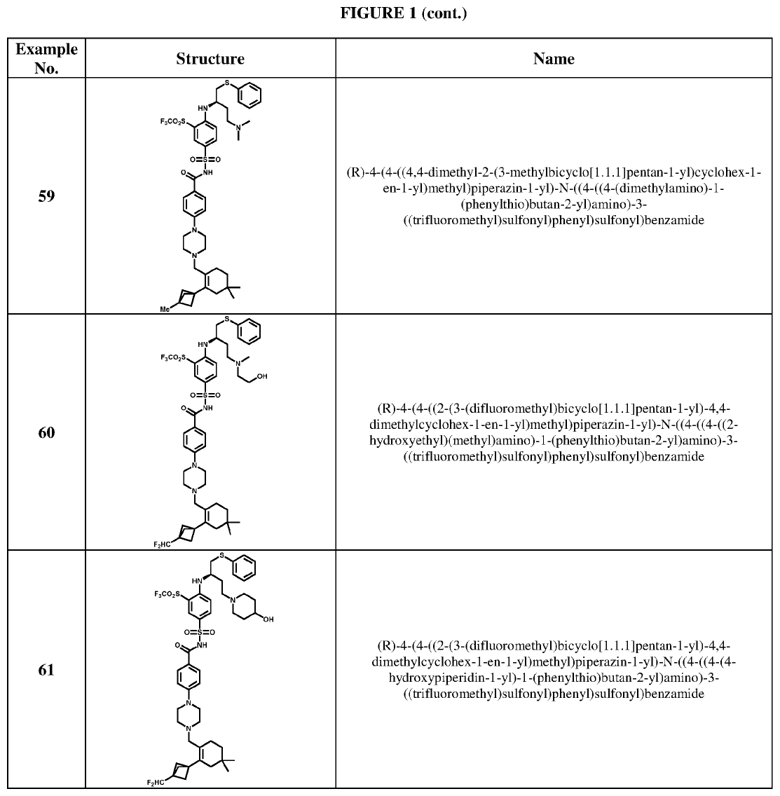

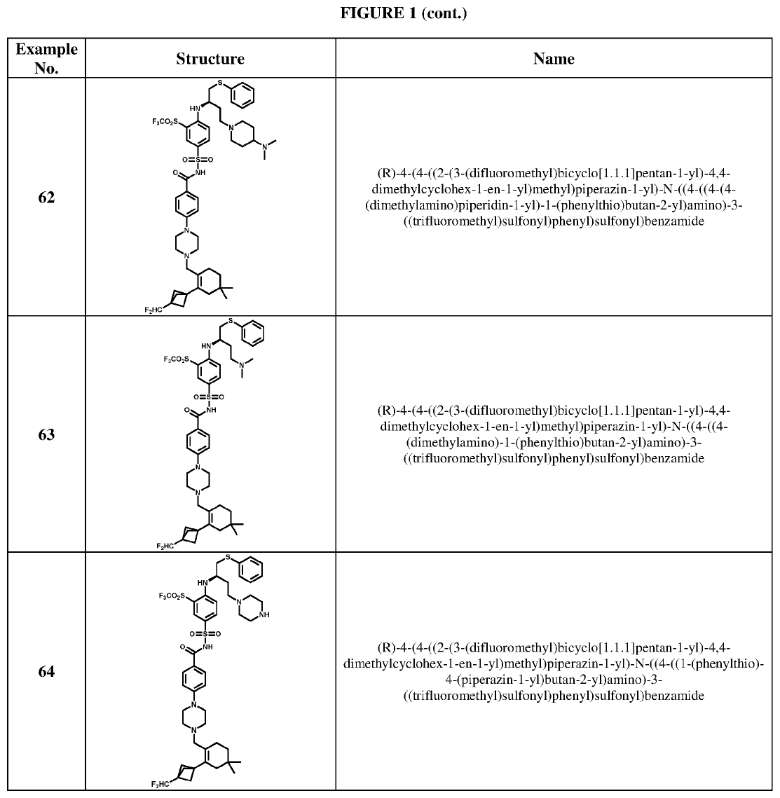

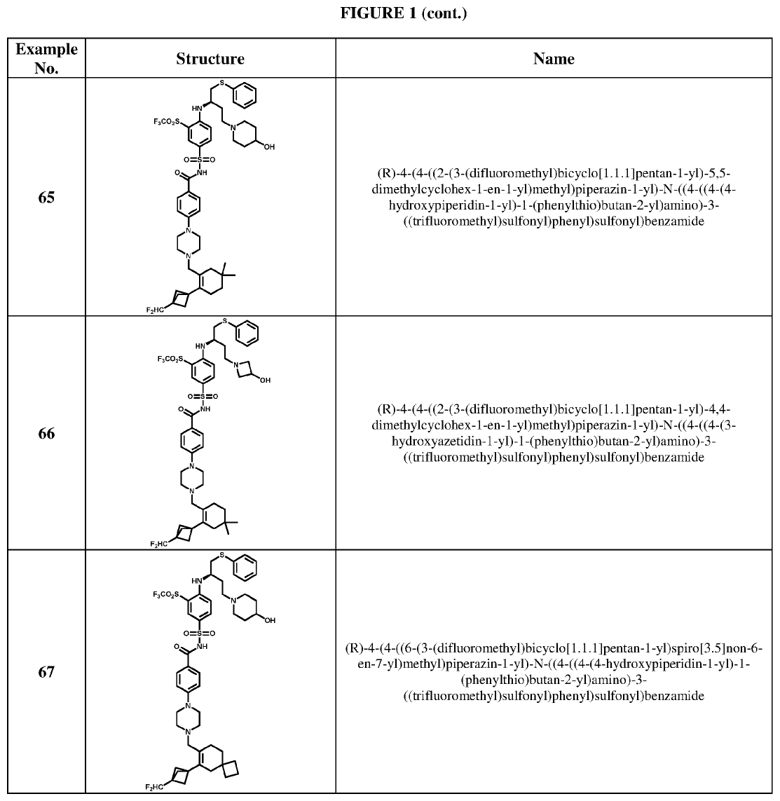

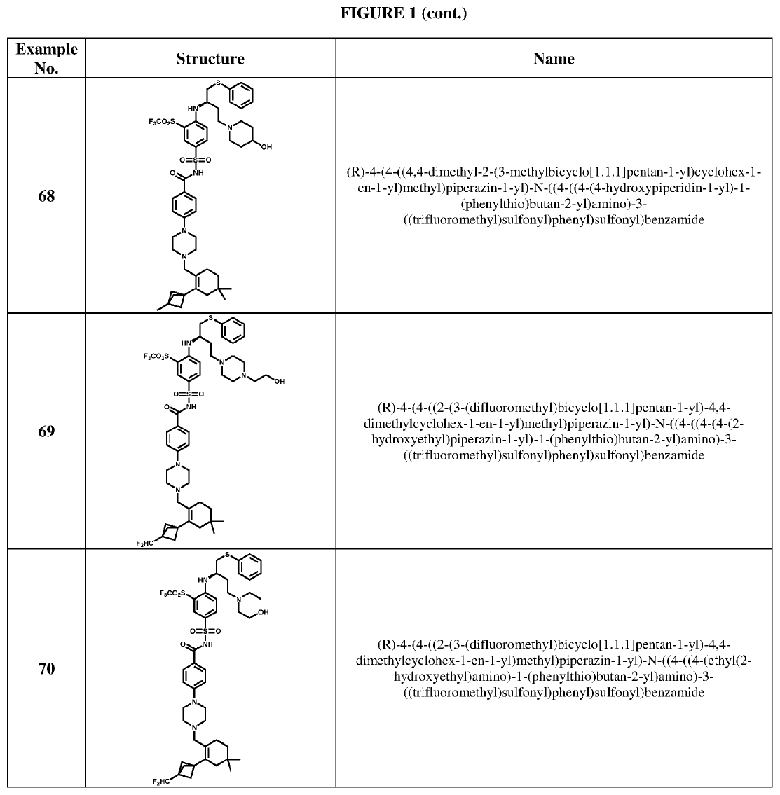

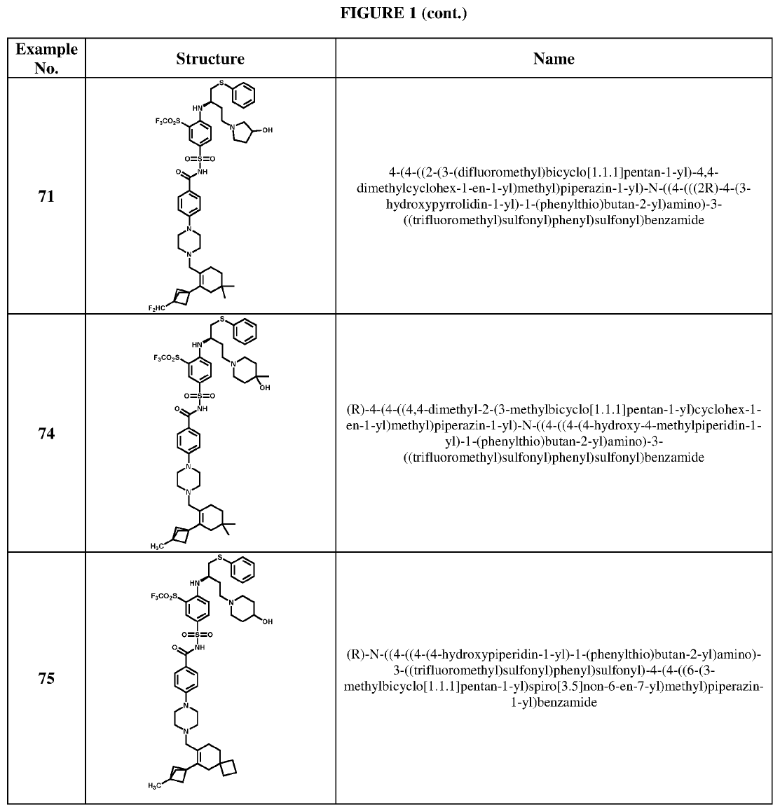

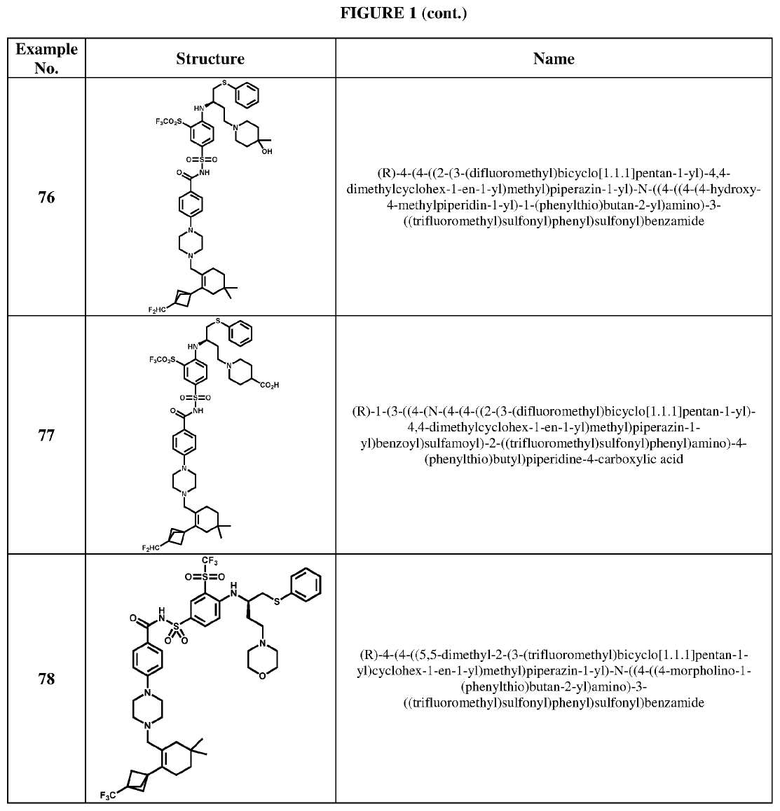

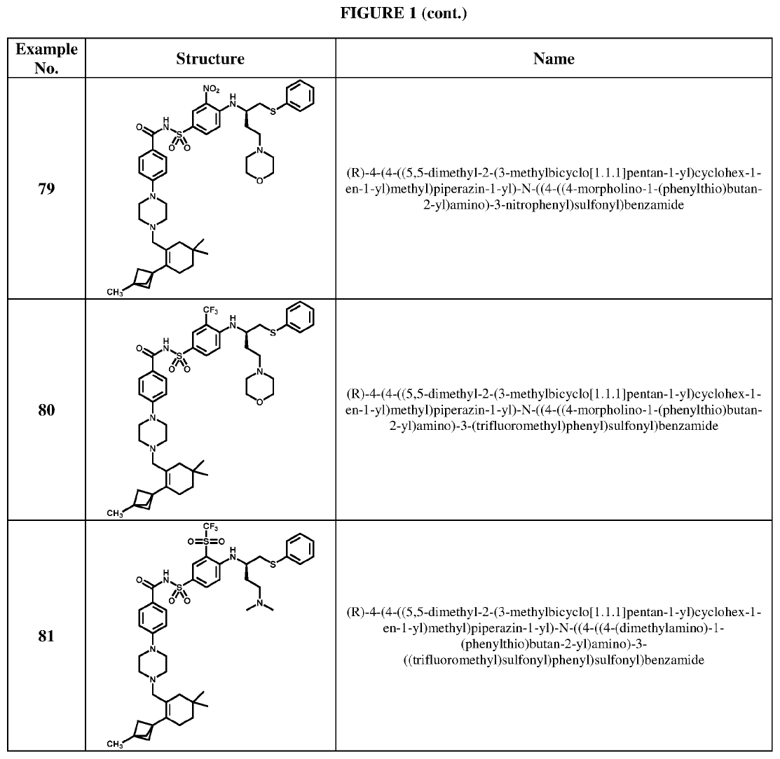

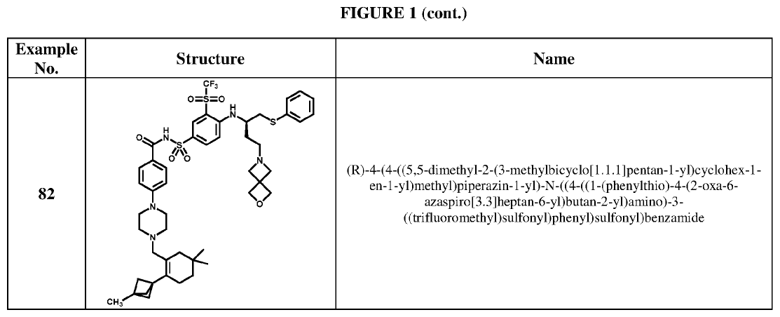

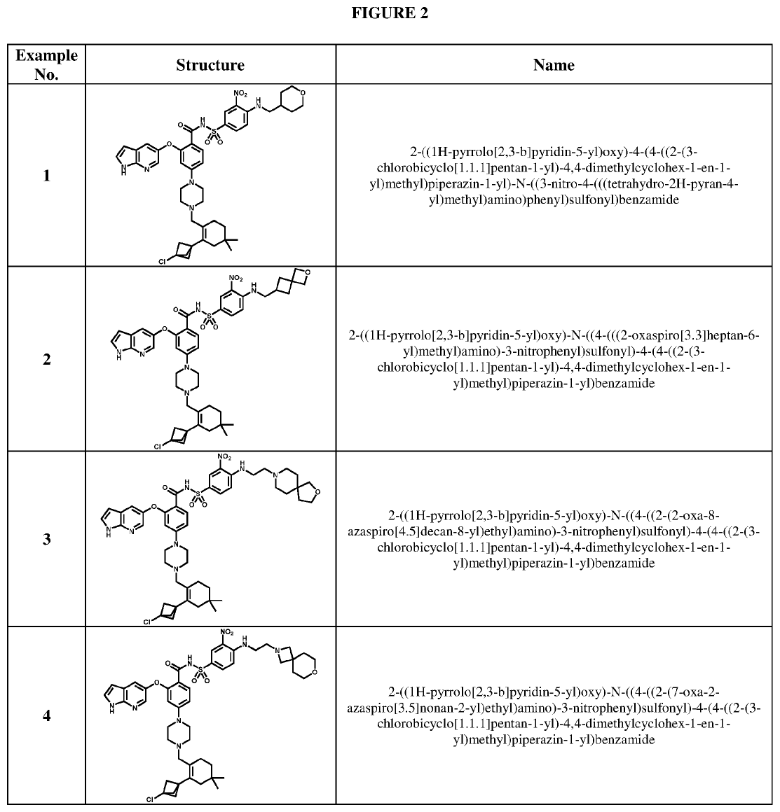

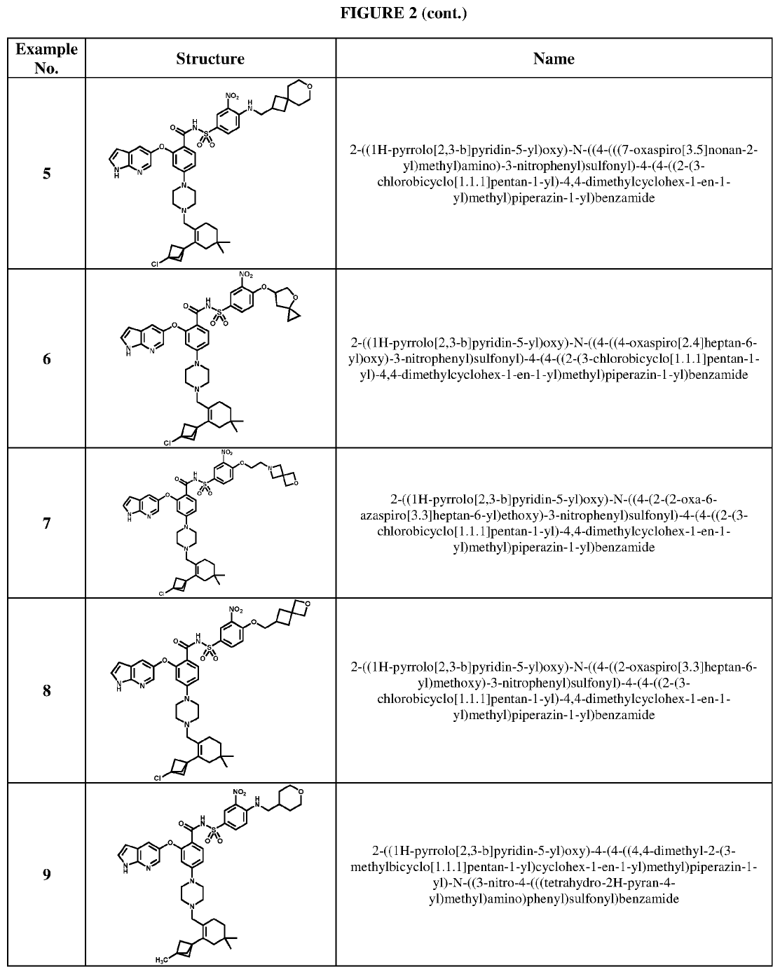

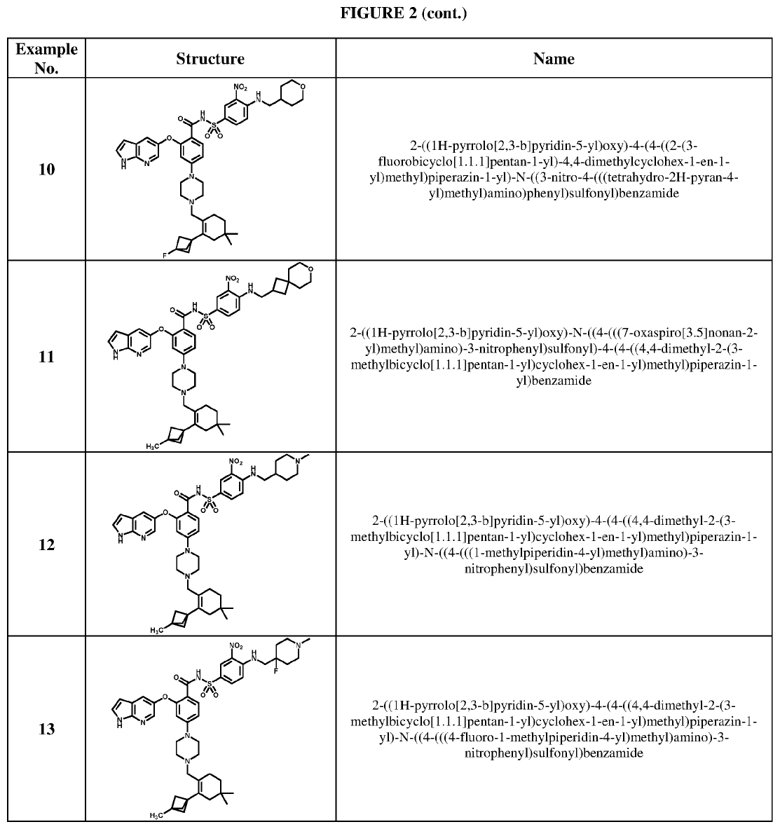

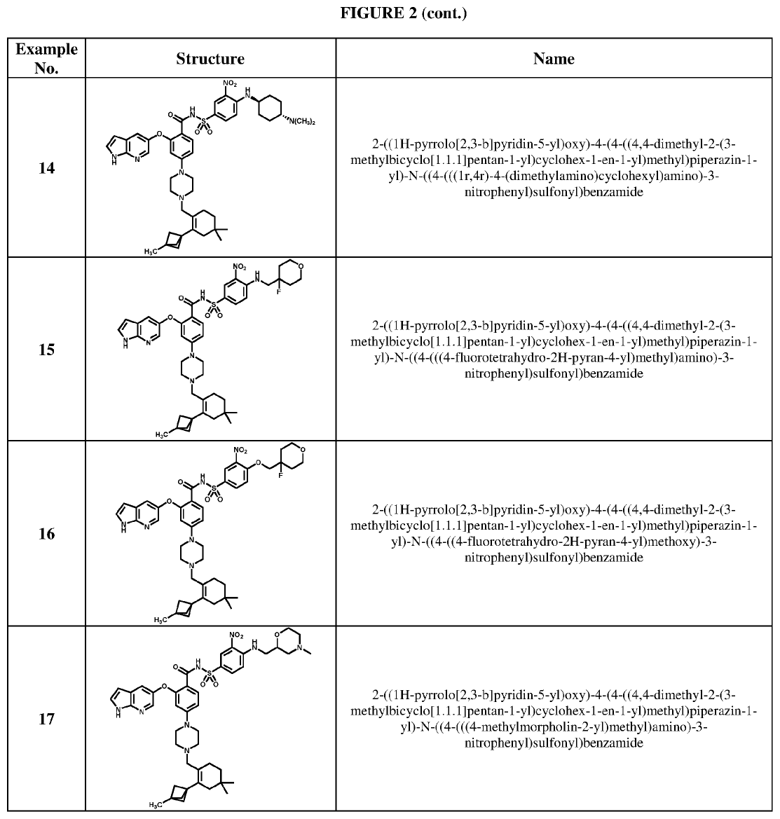

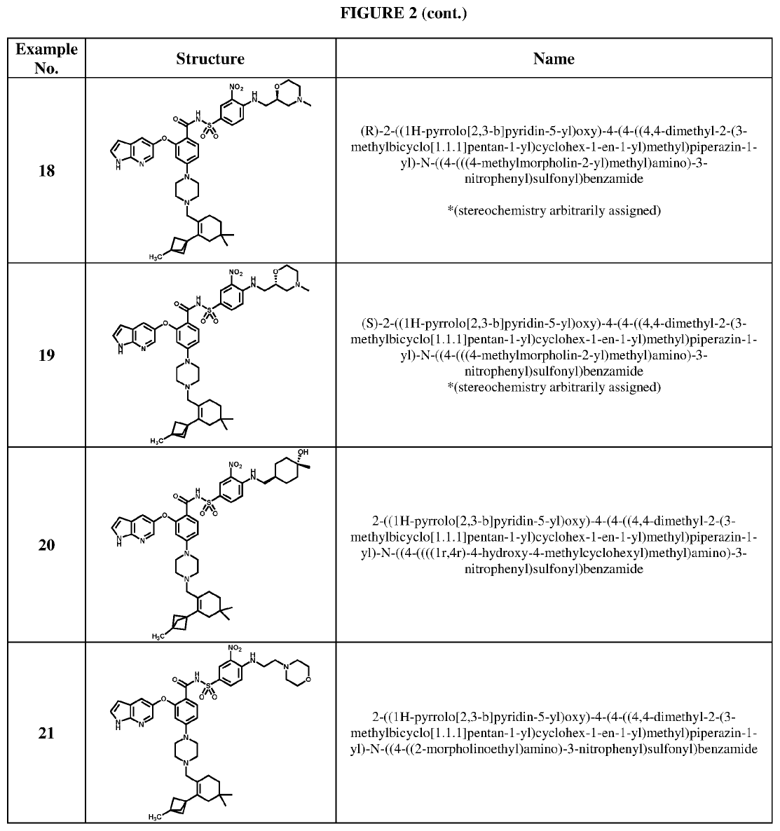

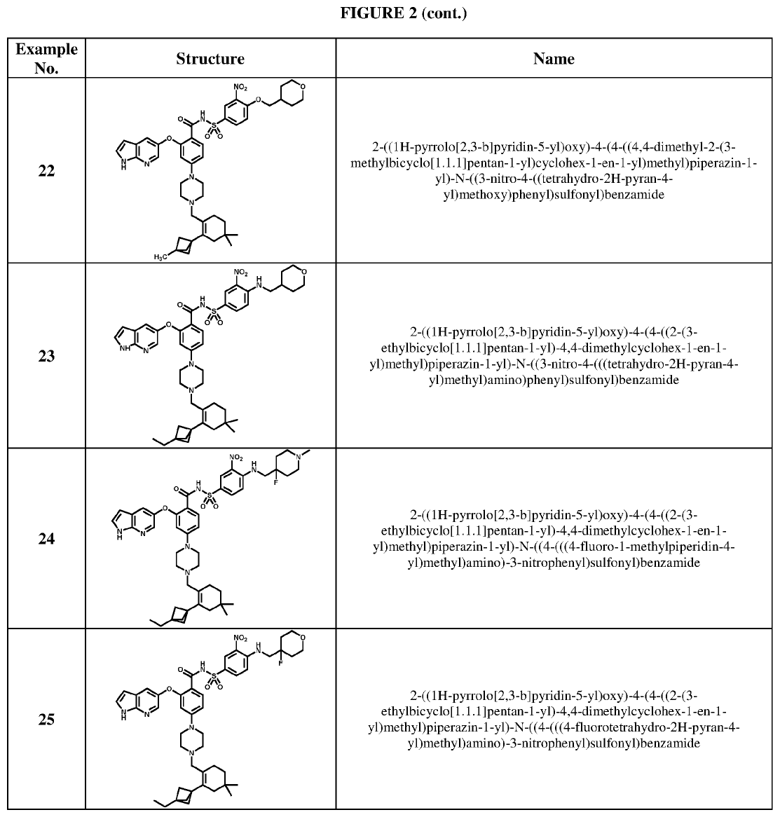

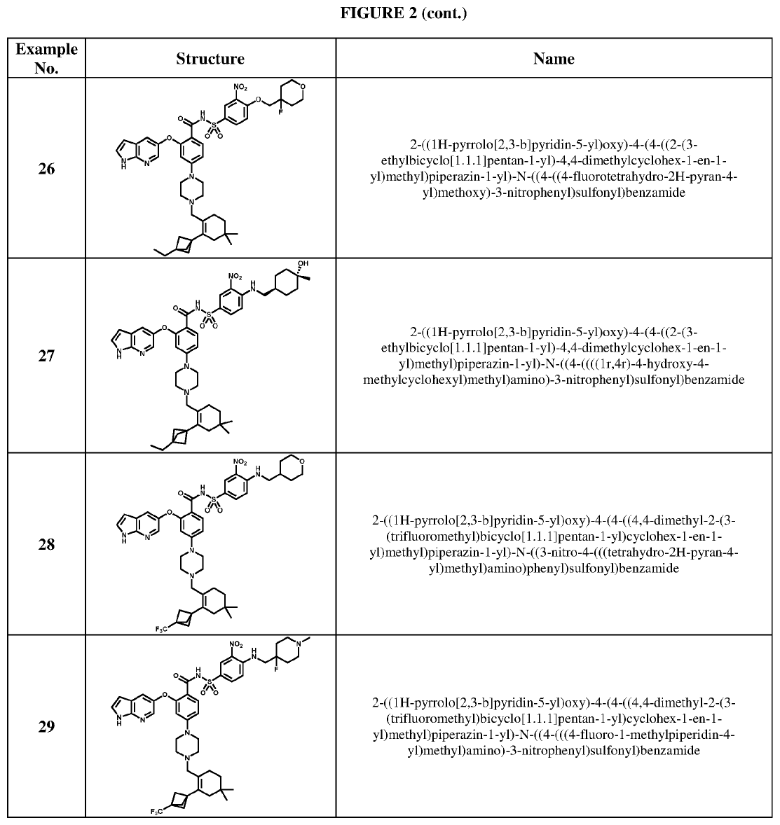

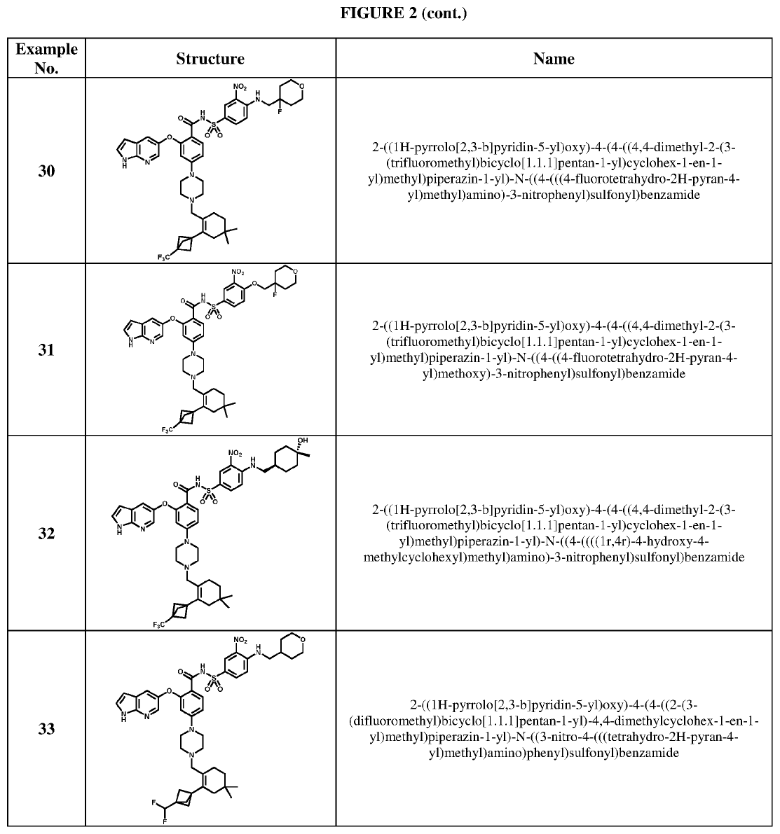

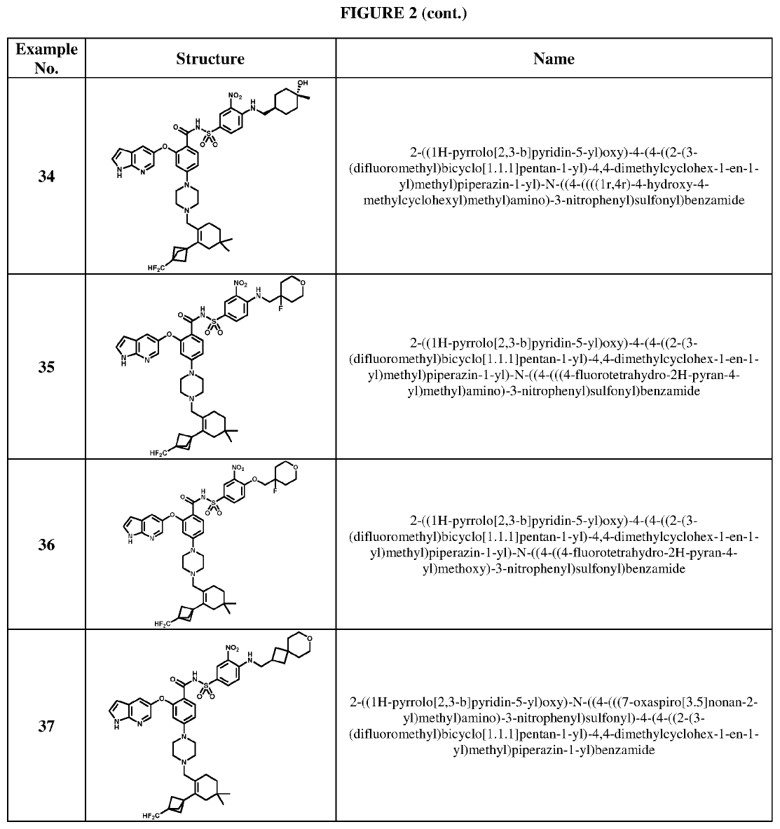

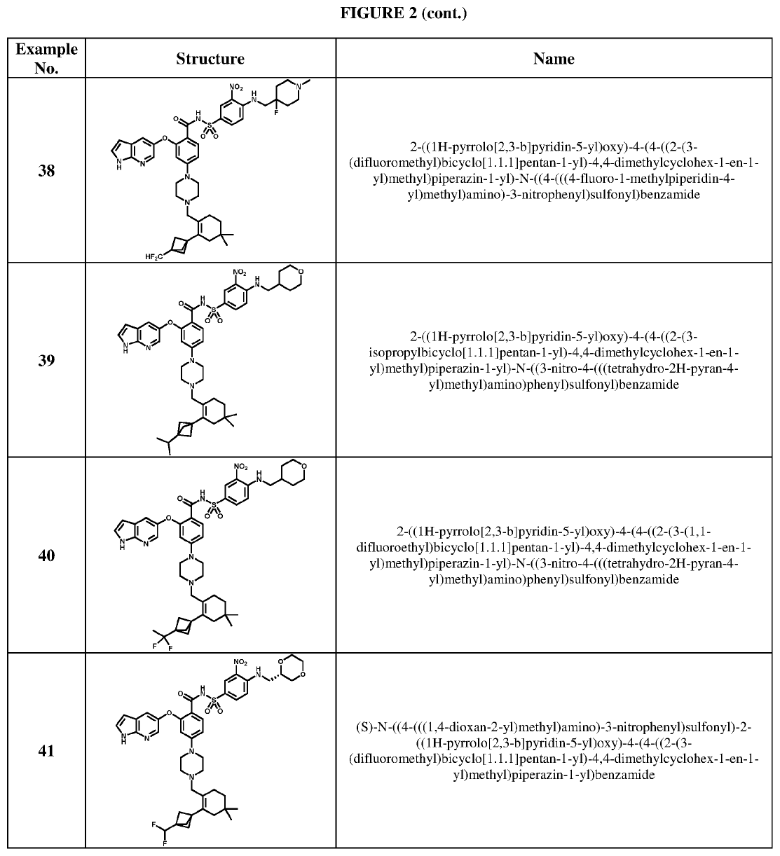

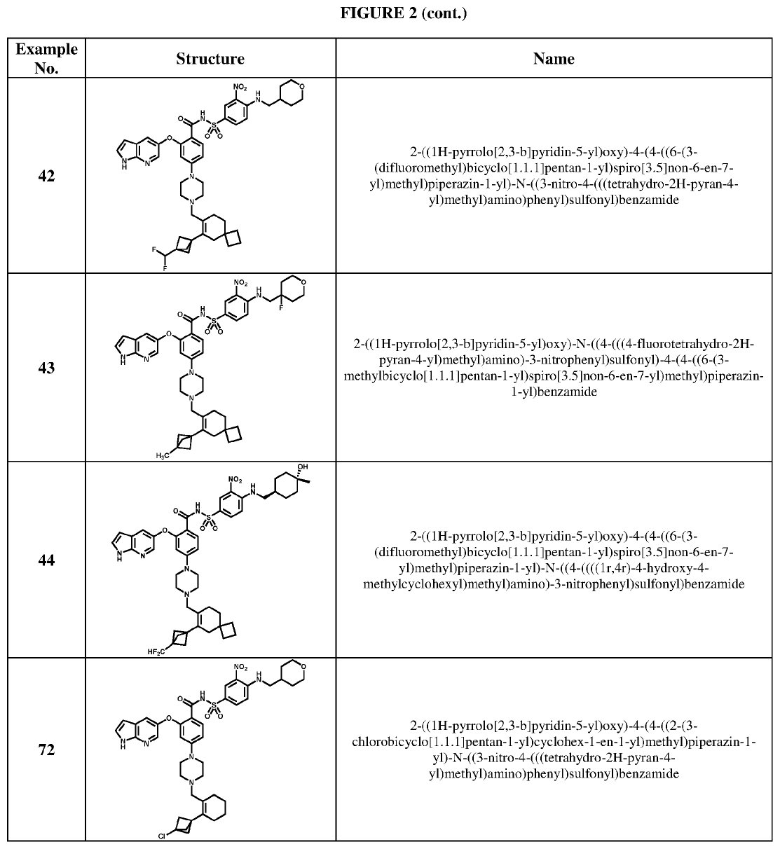

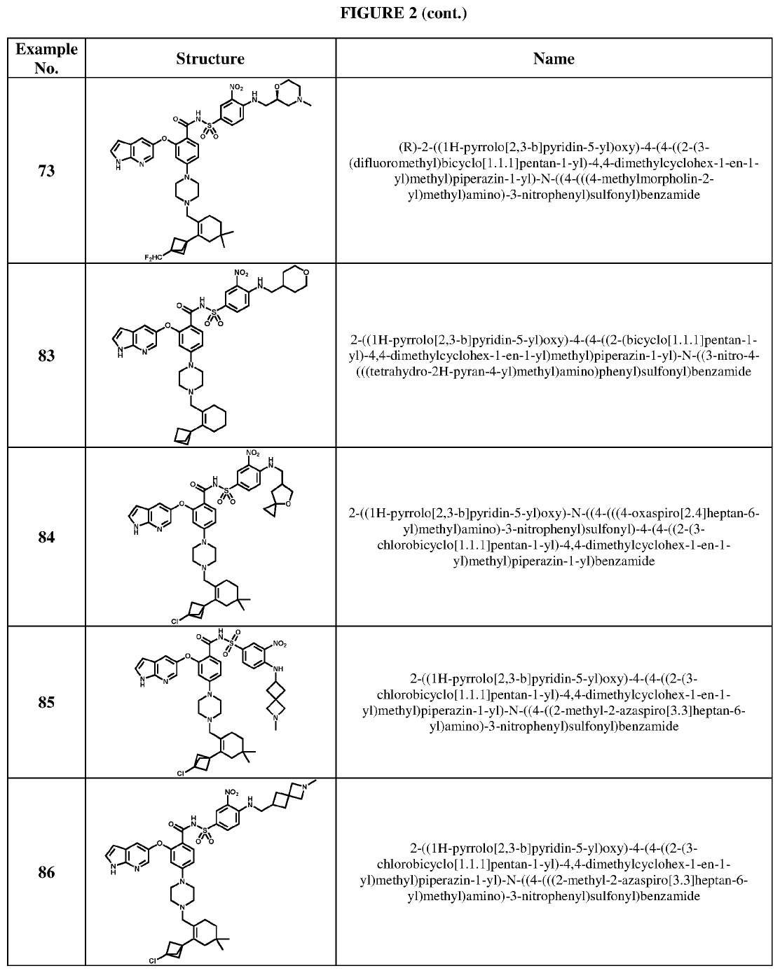

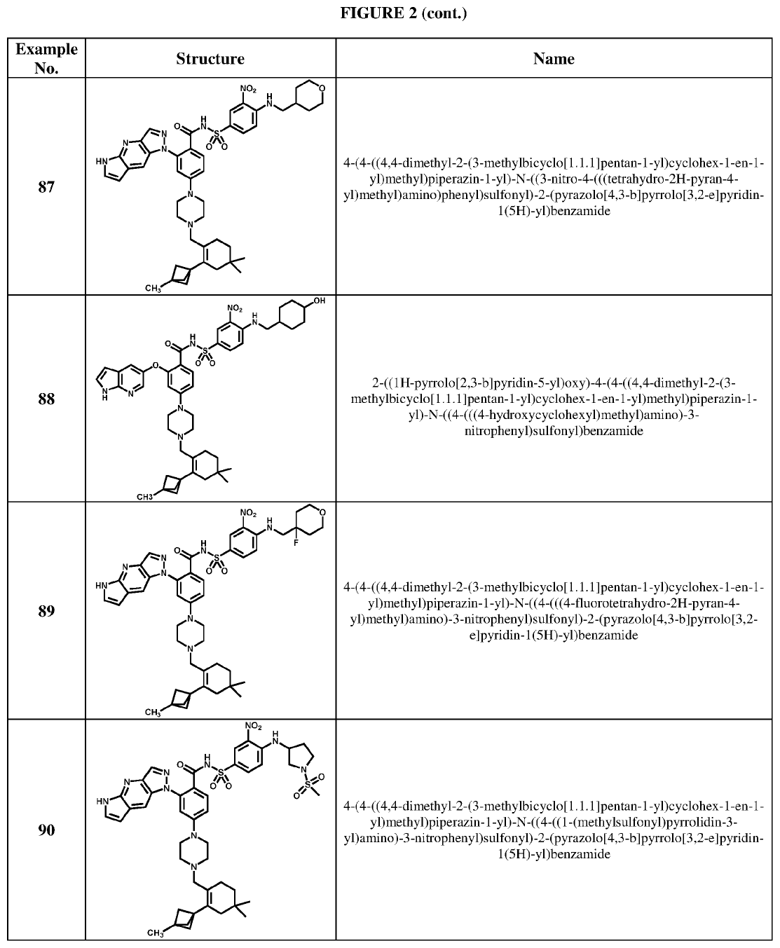

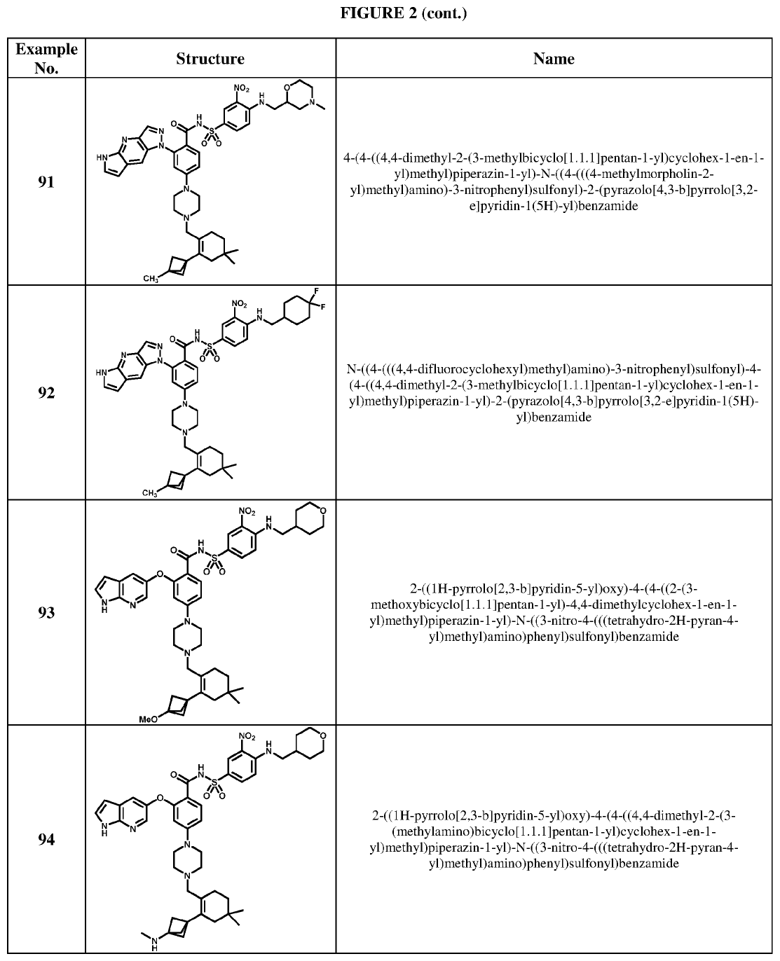

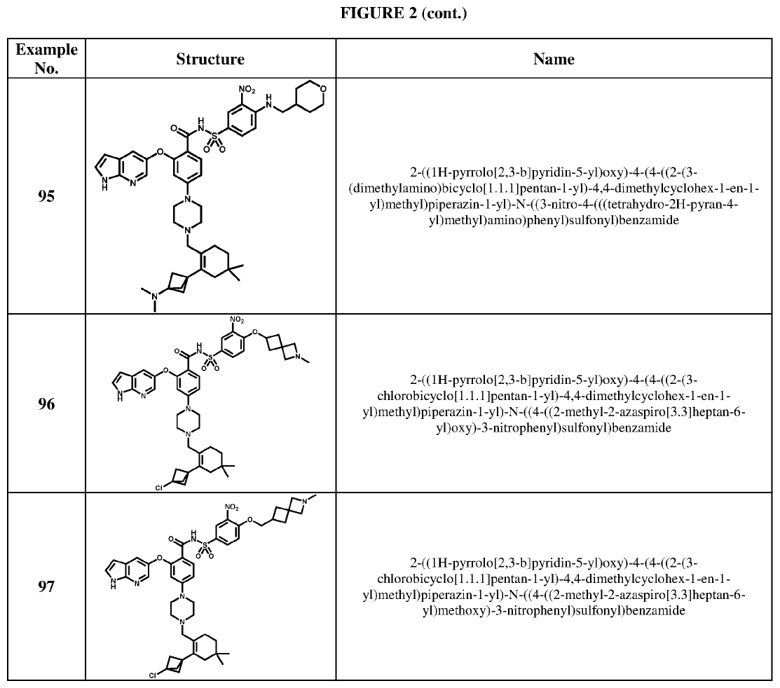

FIG. 1 provides the chemical names and structures for examples of the compounds of Formula (I) listed above in which R³ is hydrogen or halogen, along with other examples of such compounds. In an embodiment, the compound of Formula (I) is a compound selected from FIG. 1, or a pharmaceutically acceptable salt of any of the compounds listed in FIG. 1. FIG. 2 provides the chemical names and structures for examples of the compounds of Formula (I) listed above in which R³ is X—R^{3A},

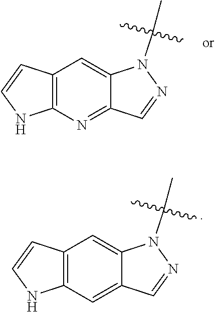 [SMILES: *n1ncc2nc3[nH]ccc3cc21 ; *n1ncc2cc3[nH]ccc3cc21]

In an embodiment, the compound of Formula (I) is a compound selected from FIG. 2, or a pharmaceutically acceptable salt of any of the compounds listed in FIG. 2.

In some embodiments, a compound of Formula (I), or a pharmaceutically acceptable salt thereof, can have increased metabolic and/or plasma stability. In some embodiments, a compound of Formula (I), or a pharmaceutically acceptable salt thereof, can be more resistant to hydrolysis and/or more resistant to enzymatic transformations. In some embodiments, a compound of Formula (I), or a pharmaceutically acceptable salt thereof, can have improved properties. A non-limiting list of example properties include, but are not limited to, increased biological half-life, increased bioavailability, increase potency, a sustained in vivo response, increased dosing intervals, decreased dosing amounts, decreased cytotoxicity, reduction in required amounts for treating disease conditions, a reduction of morbidity or mortality in clinical outcomes, decrease in or prevention of opportunistic infections, increased subject compliance and increased compatibility with other medications. In some embodiments, a compound of Formula (I), or a pharmaceutically acceptable salt thereof, can have more potent anticancer activity (for example, a lower EC₅₀ in a cell replication assay) as compared to the current standard of care (such as venetoclax). In some embodiments, a compound of Formula (I), or a pharmaceutically acceptable salt thereof, can have more potent antiviral activity (for example, a lower EC₅₀ in a HIV replicon assay) as compared to the current standard of care (such as dolutegravir).

### Synthesis

Compounds of the Formula (I), or pharmaceutically acceptable salts thereof, can be made in various ways by those skilled using known techniques as guided by the detailed teachings provided herein. For example, in an embodiment, compounds of the Formula (I) are prepared in accordance with General Scheme 1 as shown herein. In another embodiment, compounds of the Formula (I), or pharmaceutically acceptable salts thereof, can be made as described in PCT Publication Nos. WO 2019/139899, WO 2019/139900, WO 2019/139902, and WO 2019/139907, each of which is hereby incorporated herein by reference and particularly for the purpose of describing compounds of the Formula (I), pharmaceutically acceptable salts thereof, and methods of making them.

In general, the coupling reaction reactions between compounds of the general Formulae A and B to form compounds of the Formula (I) as illustrated in General Scheme 1 can be carried out in a manner similar to the reactions as described herein in the Examples, by appropriate adjustment of the reagents and conditions described in the Examples. Any preliminary reaction steps required to form starting compounds of the general Formula A and B, or other precursors, can be carried out by those skilled in the art. In General Scheme 1, R¹, R², R³ R⁴, R⁵ and m can be as described herein.

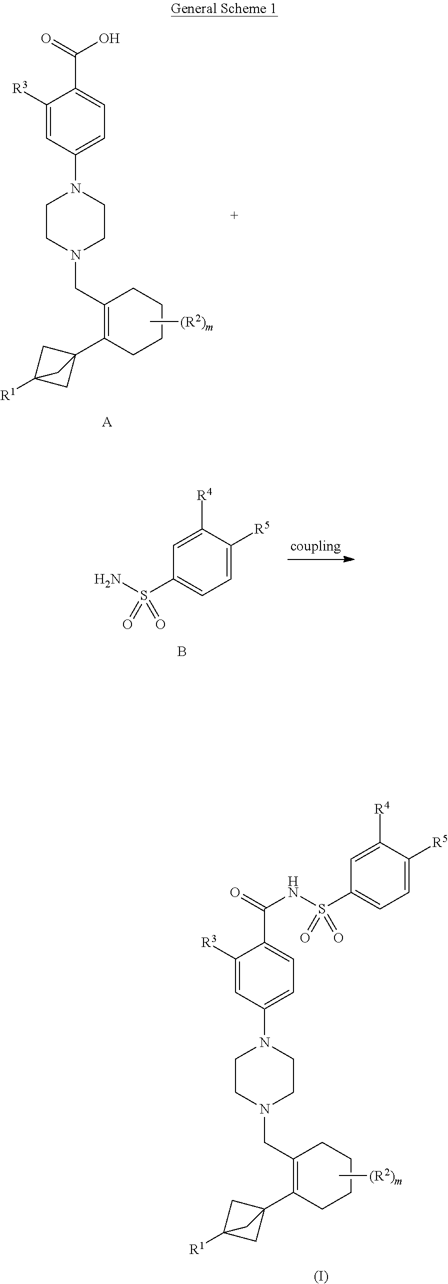

### Pharmaceutical Compositions

Some embodiments described herein relate to a pharmaceutical composition, that can include an effective amount of one or more compounds described herein (for example, a compound of Formula (I), or a pharmaceutically acceptable salt thereof) and a pharmaceutically acceptable carrier comprising albumin. In various embodiments such a pharmaceutical composition can further include another pharmaceutically acceptable carrier, a diluent, an excipient and/or combination thereof. The pharmaceutical compositions described herein can be formulated to be or to contain albumin carriers, such as albumin nanostructures, albumin microparticles or albumin nanoparticles, in which the albumin facilitates in vivo delivery of the compound of Formula (I), or a pharmaceutically acceptable salt thereof. Those skilled in the art recognize that various albumin carriers and methods of making them are known. See, e.g., M. Karimi et al., “Albumin nanostructures as advanced drug delivery systems”, Expert Opin Drug Deliv. 2016 November; 13(11): 1609-1623; see also U.S. Pat. No. 7,820,788 and PCT Publication WO 2008/109163, all of which are hereby incorporated herein by reference and particularly for the purpose of describing albumin carriers that contain active pharmaceutical ingredients (APIs), and methods of making them.

The term “pharmaceutical composition” refers to a mixture of one or more compounds of Formula (I) and/or salts as described herein with albumin and optionally other chemical components, such as diluents, excipients and/or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, albumin is a carrier that facilitates the delivery of many APIs to cells or tissues of a subject. Various types of albumin can be used to make albumin carriers, such as ovalbumin (OVA) (derived from egg white), human serum albumin (HSA), bovine serum albumin (BSA), and rat serum albumin (RSA). Various methods are known for making such albumin carriers, such as emulsion-based methods, coacervation methods, self-assembly, nanoparticle albumin-bound technology (Nab-technology) processes, gelation and spray drying. Albumin carriers can be formulated into particles having various shapes, structures and sizes, such as albumin nanoparticles, albumin microspheres, albumin-coated liposomes, albumin microbubbles, and albumin nanocapsules. The compound of Formula (I), or a pharmaceutically acceptable salt thereof, can be incorporated into the pharmaceutical composition in various ways known to those skilled in the art. For example, the compound of Formula (I), or a pharmaceutically acceptable salt thereof, can be in the form of an albumin-drug conjugate. See, e.g., M. Karimi et al., “Albumin nanostructures as advanced drug delivery systems”, Expert Opin Drug Deliv. 2016 November; 13(11): 1609-1623.

In various embodiments, the albumin and the compound of Formula (I), or a pharmaceutically acceptable salt thereof, in the pharmaceutical composition are formulated as particles. The particles can have various sizes. For example, in some embodiments the particles have an average diameter of less than 10 μm, less than 1 μm, less than 800 nm, less than 500 nm, less than 200 nm, or less than 100 nm.

The relative amounts of the albumin and the compound of Formula (I), or a pharmaceutically acceptable salt thereof in the pharmaceutical composition can vary over a broad range. For example, in an embodiment, the ratio (w/w) of the albumin to the compound of Formula (I), or a pharmaceutically acceptable salt thereof, in the pharmaceutical composition is in a range from about 1:50 to about 100:1, from about 1:10 to about 100:1, from about 1:5 to about 100:1, from about 1:1 to about 100:1, from about 1:1 to about 90:1, from about 1:1 to about 80:1, from about 1:1 to about 70:1, from about 1:1 to about 60:1, or from about 1:1 to about 50:1. In another embodiment, the ratio (w/w) of the albumin to the compound of Formula (I), or a pharmaceutically acceptable salt thereof, in the pharmaceutical composition is in a range from 1:50 to 100:1, from 1:10 to 100:1, from 1:5 to 100:1, from 1:1 to 100:1, from 1:1 to 90:1, from 1:1 to 80:1, from 1:1 to 70:1, from 1:1 to 60:1, or from 1:1 to 50:1. In another embodiment, the ratio (w/w) of the albumin to the compound of Formula (I), or a pharmaceutically acceptable salt thereof, in the pharmaceutical composition is about 1:50, about 1:40, about 1:30, about 1:20, about 1:10, about 1:1, about 10:1, about 20:1, about 30:1, about 40:1, about 50:1, about 60:1, about 70:1, about 80:1, about 90:1 or about 100:1. In another embodiment, the ratio (w/w) of the albumin to the compound of Formula (I), or a pharmaceutically acceptable salt thereof, in the pharmaceutical composition is 1:50, 1:40, 1:30, 1:20, 1:10, 1:1, 10:1, 20:1, 30:1, 40:1, 50:1, 60:1, 70:1, 80:1, 90:1 or 100:1.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Albumin particles included in the pharmaceutical composition can be made by known methods, such as emulsion-based methods, coacervation methods, self-assembly, nanoparticle albumin-bound technology (Nab-technology) processes, gelation and spray drying. Additionally, the compound of Formula (I), or a pharmaceutically acceptable salt thereof, is contained in the pharmaceutical composition in an amount effective to achieve its intended purpose. Many of the compounds of Formula (I) used in the pharmaceutical compositions disclosed herein may be provided as salts with pharmaceutically compatible counterions.

Multiple techniques of administering a pharmaceutical composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a pharmaceutical composition can be administered intravenously, e.g., by injection into a vein.

One may also administer the pharmaceutical composition in a local rather than systemic manner, for example, via injection or implantation of the compound directly into the affected area, often in a depot or sustained release formulation. Furthermore, one may administer the compound of Formula (I) in a targeted drug delivery system, for example, contained in an albumin particle coated with a tissue-specific antibody. The albumin particle will be targeted to and taken up selectively by the organ. In some cases, intranasal or pulmonary delivery to target a respiratory disease or condition may be desirable.

The pharmaceutical compositions may, if desired, be presented in a pack or dispenser device which may contain one or more unit dosage forms containing the API. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container and labeled for treatment of an indicated condition.

### Uses and Methods of Treatment

Some embodiments described herein relate to a method for treating a cancer or a tumor described herein that can include administering an effective amount of a pharmaceutical composition as described herein (which includes albumin and a compound of Formula (I), or a pharmaceutically acceptable salt thereof) to a subject having a cancer described herein. Other embodiments described herein relate to the use of an effective amount of such a pharmaceutical composition as described herein in the manufacture of a medicament for treating a cancer or a tumor described herein. Still other embodiments described herein relate to an effective amount of such a pharmaceutical composition as described herein for treating a cancer or a tumor described herein.

Some embodiments described herein relate to a method for inhibiting replication of a malignant growth or a tumor described herein that can include contacting the growth or the tumor with an effective amount of a pharmaceutical composition as described herein (which includes albumin and a compound of Formula (I), or a pharmaceutically acceptable salt thereof). Other embodiments described herein relate to the use of an effective amount of such a pharmaceutical composition in the manufacture of a medicament for inhibiting replication of a malignant growth or a tumor described herein. In some embodiments, the use can include contacting the growth or the tumor with the medicament. Still other embodiments described herein relate to an effective amount of such a pharmaceutical composition for inhibiting replication of a malignant growth or a tumor described herein.

Some embodiments described herein relate to a method for treating a cancer described herein that can include contacting a malignant growth or a tumor described herein with an effective amount of a pharmaceutical composition as described herein (which includes albumin and a compound of Formula (I), or a pharmaceutically acceptable salt thereof). Other embodiments described herein relate to the use of an effective amount of such a pharmaceutical composition in the manufacture of a medicament for treating a cancer described herein. In some embodiments, the use can include contacting the malignant growth or a tumor described herein with the medicament. Still other embodiments described herein relate to an effective amount of such a pharmaceutical composition for contacting a malignant growth or a tumor described herein, wherein the malignant growth or tumor is due to a cancer described herein.

Examples of suitable malignant growths, cancers and tumors include, but are not limited to: bladder cancers, brain cancers, breast cancers, bone marrow cancers, cervical cancers, colorectal cancers, esophageal cancers, hepatocellular cancers, lymphoblastic leukemias, follicular lymphomas, lymphoid malignancies of T-cell or B-cell origin, melanomas, myelogenous leukemias, Hodgkin's lymphoma, Non-Hodgkin's lymphoma, head and neck cancers (including oral cancers), ovarian cancers, non-small cell lung cancer, chronic lymphocytic leukemias, myelomas (including multiple myelomas), prostate cancer, small cell lung cancer, spleen cancers, polycythemia vera, thyroid cancers, endometrial cancer, stomach cancers, gallbladder cancer, bile duct cancers, testicular cancers, neuroblastomas, osteosarcomas, Ewings's tumor and Wilm's tumor.

As described herein, a malignant growth, cancer or tumor, can become resistant to one or more anti-proliferative agents. In some embodiments, a pharmaceutical composition as described herein (which includes albumin and a compound of Formula (I), or a pharmaceutically acceptable salt thereof) can be used to treat and/or ameliorate a malignant growth, cancer or tumor, that has become resistant to one or more anti-proliferative agents (such as one or more Bcl-2 inhibitors). Examples of anti-proliferative agents that a subject may have developed resistance to include, but are not limited to, Bcl-2 inhibitors (such as venetoclax, navitoclax, obatoclax, S55746, APG-1252, APG-2575 and ABT-737). In some embodiments, the malignant growth, cancer or tumor, that has become resistant to one or more anti-proliferative agents can be a malignant growth, cancer or tumor, described herein.

Some embodiments described herein relate to a method for inhibiting the activity of Bcl-2 that can include administering an effective amount of a pharmaceutical composition as described herein (which includes albumin and a compound of Formula (I), or a pharmaceutically acceptable salt thereof) to a subject and can also include contacting a cell that expresses Bcl-2 with an effective amount of such a pharmaceutical composition. Other embodiments described herein relate to the use of an effective amount of such a pharmaceutical composition in the manufacture of a medicament for inhibiting the activity of Bcl-2 in a subject or, in the manufacture of a medicament for inhibiting the activity of Bcl-2, wherein the use comprises contacting a cell that expresses Bcl-2. Still other embodiments described herein relate to an effective amount of such a pharmaceutical composition for inhibiting the activity of Bcl-2 in a subject; or for inhibiting the activity of Bcl-2 by contacting a cell that expresses Bcl-2.

In some embodiments, the Bcl protein inhibitor of Formula (I) can be a selective Bcl-2 inhibitor, a selective Bcl-X_{L} inhibitor, a selective Bcl-W inhibitor, a selective Mcl-1 inhibitor or a selective Bcl-2A1 inhibitor. In some embodiments, the Bcl protein inhibitor of Formula (I) can inhibit more than one Bcl protein. In some embodiments, the Bcl protein inhibitor can be an inhibitor of the activity of Bcl-2 and one of Bcl-X_{L}, Bcl-W, Mcl-1 and Bcl-2A1. In some embodiments, the Bcl protein inhibitor can be an inhibitor of the activity of Bcl-X_{L} and one of Bcl-W, Mcl-1 and Bcl-2A1. In some embodiments, the Bcl protein inhibitor of Formula (I) can inhibit both Bcl-2 and Bcl-X_{L}.

In some embodiments, a pharmaceutical composition as described herein (which includes albumin and a compound of Formula (I), or a pharmaceutically acceptable salt thereof) can be in a method or use described herein in combination with another Bcl protein inhibitor, e.g., venetoclax, navitoclax, obatoclax, ABT-737, 555746, AT-101, APG-1252, APG-2575, AMG176 or AZD5991, or a combination of any of the foregoing. Such methods and uses include simultaneous and sequential administrations of the multiple Bcl protein inhibitors to the subject.

Several known Bcl-2 inhibitors can cause one or more undesirable side effects in the subject being treated. Examples of undesirable side effects include, but are not limited to, thrombocytopenia, neutropenia, anemia, diarrhea, nausea and upper respiratory tract infection. In some embodiments, a pharmaceutical composition as described herein (which includes albumin and a compound of Formula (I), or a pharmaceutically acceptable salt thereof) can decrease the number and/or severity of one or more side effects associated with administration of known Bcl-2 inhibitors. In some embodiments, such a pharmaceutical composition can result in a severity of a side effect (such as one of those described herein) that is at least 25% less than compared to the severity of the same side effect experienced by a subject receiving a known Bcl-2 inhibitors (such as venetoclax, navitoclax, obatoclax, ABT-737, 555746, AT-101, APG-1252 and APG-2575). In some embodiments, such a pharmaceutical composition results in a number of side effects that is at least 25% less than compared to the number of side effects experienced by a subject receiving a known Bcl-2 inhibitors (for example, venetoclax, navitoclax, obatoclax, ABT-737, 555746, AT-101, APG-1252 and APG-2575). In some embodiments, such a pharmaceutical composition results in a severity of a side effect (such as one of those described herein) that is less in the range of about 10% to about 30% compared to the severity of the same side effect experienced by a subject receiving a known Bcl-2 inhibitors (for example, venetoclax, navitoclax, obatoclax, ABT-737, 555746, AT-101, APG-1252 and APG-2575). In some embodiments, such a pharmaceutical composition results in a number of side effects that is in the range of about 10% to about 30% less than compared to the number of side effects experienced by a subject receiving a known Bcl-2 inhibitors (for example, venetoclax, navitoclax, obatoclax, ABT-737, S55746, APG-1252 and APG-2575).

The pharmaceutical composition as described herein (which includes albumin and a compound of Formula (I), or a pharmaceutically acceptable salt thereof) that can be used to treat, ameliorate and/or inhibit the replication of a cancer, malignant growth, or tumor wherein inhibiting the activity of Bcl-2 is beneficial is provided in any of the embodiments described above under the heading titled “Pharmaceutical Compositions” For example, in various embodiments, the methods and uses described above in the Uses and Methods of Treatment section of this disclosure are carried out in the described manner (generally involving cancer, malignant growth, and/or tumor) using a compound of Formula (I) in which R³ is hydrogen or halogen, or a pharmaceutically acceptable salt thereof.

In other embodiments, the methods and uses described above in the Uses and Methods of Treatment section are carried out in the described manner (generally involving cancer, malignant growth, and/or tumor) using a compound of Formula (I) in which R³ is X—R^{3A},

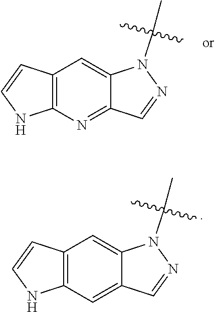 [SMILES: *n1ncc2nc3[nH]ccc3cc21 ; *n1ncc2cc3[nH]ccc3cc21]

In other embodiments, the methods and uses described above in the Uses and Methods of Treatment section of this disclosure are carried out in the described manner (generally involving cancer, malignant growth, and/or tumor) using a compound of Formula (I) in which R³ is X—R^{3A},

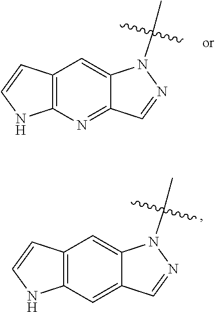 [SMILES: *n1ncc2nc3[nH]ccc3cc21 ; *n1ncc2cc3[nH]ccc3cc21]

and in which X^{t} and X² are —NH—.

In other embodiments, the methods and uses described above in the Uses and Methods of Treatment section are carried out in the described manner (generally involving cancer, malignant growth, and/or tumor) using a compound of Formula (I) in which R¹ is selected from the group consisting of hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamine and an unsubstituted di-C₁-C₆ alkylamine, with the proviso that R¹ is not —CH₂F, —CHF₂ or —CF₃; R³ is X—R^{3A},

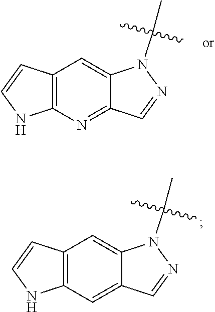 [SMILES: *n1ncc2nc3[nH]ccc3cc21 ; *n1ncc2cc3[nH]ccc3cc21]

nd X¹ and X² are —NH—.

In other embodiments, the methods and uses described above in the Uses and Methods of Treatment section are carried out in the described manner (generally involving cancer, malignant growth, and/or tumor) using a compound of Formula (I) in which R¹ is selected from the group consisting of hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamine and an unsubstituted di-C₁-C₆ alkylamine; R³ is X—R^{3A},

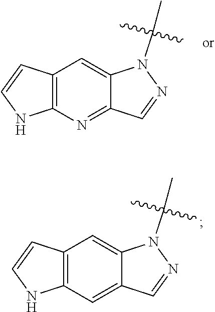 [SMILES: *n1ncc2nc3[nH]ccc3cc21 ; *n1ncc2cc3[nH]ccc3cc21]

and X¹ and X² are —O—.

In other embodiments, the methods and uses described above in the Uses and Methods of Treatment section are carried out in the described manner (generally involving cancer, malignant growth, and/or tumor) using a compound of Formula (I) in which R¹ is —CH₂F, —CHF₂ or —CF₃; R³ is X—R^{3A},

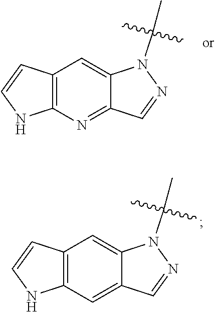 [SMILES: *n1ncc2nc3[nH]ccc3cc21 ; *n1ncc2cc3[nH]ccc3cc21]

and X¹ and X² are —NH—.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical composition that contains it, that elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer), a therapeutically effective amount is that amount that alleviates or eliminates cough, shortness of breath and/or pain. As another example, an effective amount, or a therapeutically effective amount of a Bcl-2 inhibitor is the amount which results in the reduction in Bcl-2 activity and/or an increase in apoptosis. The reduction in Bcl-2 activity is known to those skilled in the art and can be determined by the analysis of Bcl-2 binding and relatives levels of cells undergoing apoptosis.

The amount of the pharmaceutical composition as described herein (which includes albumin and a compound of Formula (I), or a pharmaceutically acceptable salt thereof) required for use in treatment will vary not only with the particular pharmaceutical composition selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compound of Formula (I), or pharmaceutically acceptable salts thereof, can be determined by comparing their in vitro activity and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### Intermediate 1

### 3-Chloro-N-methoxy-N-methylbicyclo[1.1.1]pentane-1-carboxamide

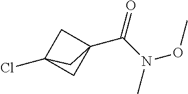

To a stirred solution of 3-chlorobicyclo[1.1.1]pentane-1-carboxylate (10.0 g, 62.3 mmol) and N,O-dimethylhydroxylamine hydrochloride (12.15 g, 124.5 mmol) in anhydrous THF (200 mL) at −78° C. was added i-PrMgCl (2 M in THF, 124.5 mL, 249 mmol). The temperature was then raised to −50° C. and stirred for 2 h. The reaction was quenched with sat. aq. NH₄Cl and extracted with EtOAc (3×150 mL). The combined organic layers were washed with water, brine, dried over Na₂SO₄, filtered, and concentrated. The crude product was purified by column chromatography (SiO₂, EtOAc/pet. ether) to provide Intermediate 1 (7.30 g, 62%) as an oil. ¹H NMR (300 MHz, CDCl₃) δ 3.67 (s, 3H), 3.18 (s, 3H), 2.47 (s, 6H).

### Intermediate 2

### tert-Butyl 2-(1H-pyrrolo[2,3-b]pyridin-5-yloxy)-4-(piperazin-1-yl)benzoate

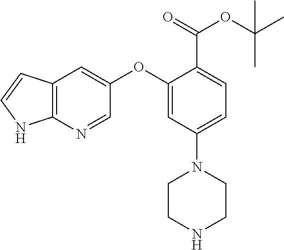 [SMILES: CC(C)(C)OC(=O)c1ccc(N2CCNCC2)cc1Oc1cnc2[nH]ccc2c1]

A solution of tert-butyl 2-(1H-pyrrolo[2,3-b]pyridin-5-yloxy)-4-fluorobenzoate (3.5 g, 10.67 mmol) in DMSO (35 mL) was treated with piperazine (2.33 ml, 32.0 mmol) at rt and stirred at 100° C. for 4 h. The reaction was cooled to rt and water (50 mL) was added. The mixture was extracted with EtOAc (3×50 ml) and the organic layers were concentrated and triturated with n-pentane to provide Intermediate 2 (3.0 g, 71%) as a white solid. LC/MS (ESI) m/z 395.5 [M+H]⁺.

### Intermediate 3

### 4-(2-oxaspiro[3.3]heptan-6-ylmethylamino)-3-nitrobenzenesulfonamide

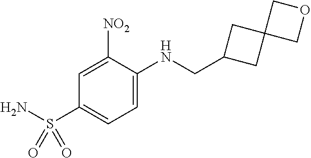 [SMILES: NS(=O)(=O)c1ccc(NCC2CC3(COC3)C2)c([N+](=O)[O-])c1]

A solution of 4-chloro-3-nitrobenzenesulfonamide (200 mg, 0.85 mmol) in CH₃CN (8 mL) was treated with (2-oxaspiro[3.3]heptan-6-yl)methanamine (129 mg, 1.01 mmol) and DIPEA (0.5 mL 2.95 mmol). The mixture was heated to 90° C. and stirred for 16 h. The reaction was cooled to rt, diluted with EtOAc, and washed with water and brine. The organic layer was dried over Na₂SO₄, filtered and concentrated. The crude product was purified by column chromatography (SiO₂, EtOAc/hexanes) to afford Intermediate 3 (120 mg, 43%) as a yellow solid. ¹H NMR (400 MHz, DMSO-d₆) δ 8.47-8.43 (m, 2H), 7.83-7.80 (m, 1H), 7.30 (br s, 2H), 7.22 (d, J=9.6 Hz, 1H), 4.56 (s, 2H), 4.49 (s, 2H), 3.42-3.38 (m, 2H), 2.45-2.39 (m, 1H), 2.33-2.27 (m, 2H), 1.99-1.94 (m, 2H).

### Intermediate 4

### 4-(2-(2-oxa-8-azaspiro[4.5]decan-8-yl)ethylamino)-3-nitrobenzenesulfonamide

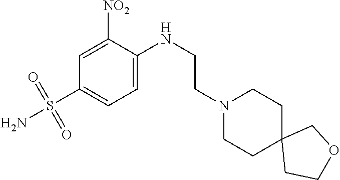 [SMILES: NS(=O)(=O)c1ccc(NCCN2CCC3(CCOC3)CC2)c([N+](=O)[O-])c1]

Step 1: A solution of 2-oxa-8-azaspiro[4.5] decane hydrochloride (500 mg, 2.81 mmol) in CH₃CN (20 mL) was treated with tert-butyl-2-bromoethylcarbamate (700 mg, 3.12 mmol) and K₂CO₃ (1.55 g, 11.24 mmol) and heated to 80° C. for 16 h. The reaction was concentrated, diluted with water (20 mL), and extracted with EtOAc (3×20 mL). The combined organic layers were dried over Na₂SO₄, filtered and concentrated. The residue was purified by column chromatography (SiO₂, EtOAc/pet. ether) to afford tert-butyl-2-(2-oxa-8-azaspiro[4.5]decan-8-yl)ethylcarbamate (Intermediate 4-1) (500 mg, 62%) as an oil. ¹H NMR (300 MHz, DMSO-d₆) δ 6.62 (br s, 1H), 3.70 (t, J=6.9 Hz, 2H), 3.40 (s, 2H), 3.04-2.98 (m, 2H), 2.40-2.25 (m, 4H), 1.64 (t, J=7.5 Hz, 2H), 1.56-1.40 (m, 4H), 1.37 (s, 9H), 1.24 (s, 2H).

Step 2: To a stirred solution of Intermediate 4-1 (500 mg, 1.76 mmol) in DCM (20 mL) was added HCl (4 M in dioxane, 10 mL) at 0° C. The reaction was warmed to rt, stirred for 2 h, concentrated and triturated with Et₂O to afford 2-(2-oxa-8-azaspiro[4.5]decan-8-yl)ethanamine dihydrochloride (Intermediate 4-2) (300 mg, 66%) as an off white solid which was used for the next step without further purification. ¹H NMR (300 MHz, DMSO-d₆) δ 10.84 (br s, 1H), 8.38 (br s, 3H), 3.85-3.70 (m, 2H), 3.59-3.40 (m, 8H), 3.12-2.90 (m, 2H), 2.05-1.60 (m, 6H).

Step 3: A solution of Intermediate 4-2 (300 mg, 1.17 mmol) in CH₃CN (15 mL) was treated with 4-chloro-3-nitrobenzenesulfonamide (276 mg, 1.17 mmol) followed by DIPEA (0.82 mL, 4.68 mmol) and then heated to 80° C. After 16 h, the reaction was cooled to rt and concentrated. The crude product was purified by column chromatography (SiO₂, MeOH (0.1% triethylamine)/DCM) to afford Intermediate 4 (300 mg, 66%) as a yellow solid. LC/MS (ESI) m/z 385.3 [M+H]⁺.

### Intermediate 5

### 2-(7-oxa-2-azaspiro[3.5]nonan-2-yl)ethanamine dihydrochloride

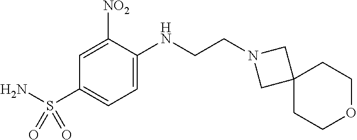 [SMILES: NS(=O)(=O)c1ccc(NCCN2CC3(CCOCC3)C2)c([N+](=O)[O-])c1]

Step 1: tert-butyl 2-(7-oxa-2-azaspiro[3.5]nonan-2-yl)ethylcarbamate (Intermediate 5-1) was prepared following the procedure described in Step 1 for Intermediate 4 using 7-oxa-2-azaspiro[3.5] nonane hemioxalic acid in place of 2-oxa-8-azaspiro[4.5] decane hydrochloride ¹H NMR (300 MHz, DMSO-d₆) δ 6.94 (br s, 1H), 3.74 (br s, 4H), 3.51-3.42 (m, 4H), 3.10 (br s, 4H), 1.76 (br s, 4H), 1.39 (s, 9H).

Step 2: 2-(7-oxa-2-azaspiro[3.5]nonan-2-yl)ethanamine dihydrochloride (Intermediate 5-2) was prepared following the procedure described in Step 2 for Intermediate 4 using Intermediate 5-1 in place of Intermediate 4-1. ¹H NMR (300 MHz, DMSO-d₆) δ 11.42 (br s, 1H), 8.3 (br s, 3H), 4.05-3.99 (m, 2H), 3.92-3.86 (m, 2H), 3.57-3.54 (m, 4H), 3.49-3.40 (m, 4H), 3.10-3.05 (m, 2H), 1.88 (br s, 2H), 1.72 (br s, 2H).

Step 3: A solution of Intermediate 5-2 (250 mg, 1.03 mmol) in CH₃CN (13 mL) was treated with 4-fluoro-3-nitrobenzenesulfonamide (226.8 mg, 1.03 mmol) followed by triethylamine (0.58 mL, 4.12 mmol) at rt. After 16 h, the reaction was concentrated to afford the crude product, which was purified by column chromatography (SiO₂, MeOH (containing 7N NH₃)/DCM) to obtain Intermediate 5 (200 mg, 52%) as a yellow solid. LC/MS (ESI) m/z 371.3 [M+H]⁺.

### Intermediate 6

### 4-(7-Oxaspiro[3.5]nonan-2-yl-methylamino)-3-nitrobenzenesulfonamide

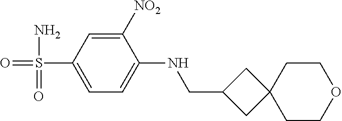 [SMILES: NS(=O)(=O)c1ccc(NCC2CC3(CCOCC3)C2)c([N+](=O)[O-])c1]

A solution of 7-oxaspiro[3.5]nonan-2-yl-methanamine (100 mg, 0.64 mmol) in THF (2 mL) was treated with 4-fluoro-3-nitrobenzenesulfonamide (157.6 mg, 0.72 mmol) and Et₃N (0.18 mL, 1.29 mmol) and the mixture was stirred at rt. After 16 h, the reaction was concentrated, and the residue was purified by column chromatography (SiO₂, MeOH/DCM) to provide Intermediate 6 (126 mg, 55%) as a yellow solid. LC/MS (ESI) m/z 356.1 [M+H]⁺.

### Intermediate 7

### 4-((4-oxaspiro[2.4]heptan-6-yl)oxy)-3-nitrobenzenesulfonamide

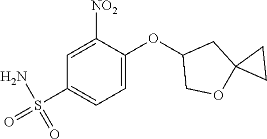 [SMILES: NS(=O)(=O)c1ccc(OC2COC3(CC3)C2)c([N+](=O)[O-])c1]

Step 1: To a stirred solution of 1-(3-hydroxy-2-(tetrahydro-2H-pyran-2-yloxy)propyl)cyclopropanol (prepared according to CN106565706) and triphenyl phosphine (9.10 g, 34.7 mmol) in THF (50 mL), was added diethyl azodicarboxylate (DEAD) (5.44 mL, 34.7 mmol) dropwise at rt. After 16 h, the reaction mixture was quenched with H₂O (50 mL) and extracted with EtOAc (3×50 mL). The combined organic layers were washed with water (50 mL), dried over Na₂SO₄ and concentrated. The crude product was purified by column chromatography (SiO2, EtOAc/pet. ether) to obtain 6-(tetrahydro-2H-pyran-2-yloxy)-4-oxaspiro[2.4]heptane (Intermediate 7-1) (3.2 g, 69% yield) as a clear yellow oil. ¹H NMR (400 MHz, CDCl₃) δ 4.65-4.63 (m, 1H), 4.59-4.56 (m, 1H), 4.02-3.85 (m, 3H), 3.53-3.48 (m, 1H), 2.25-1.95 (m, 2H), 1.89-1.76 (m, 1H), 1.72-1.68 (m, 1H), 1.62-1.49 (m, 4H), 0.92-0.89 (m, 1H), 0.81-0.75 (m, 1H), 0.65-0.53 (m, 1H), 0.48-0.39 (m, 1H).

Step 2: To a stirred solution of Intermediate 7-1 (3.2 g, 16.1 mmol) in MeOH (32 mL) was added pyridinium p-toluenesulfonate (811 mg, 3.23 mmol) and stirred at 40° C. for 5 h. The reaction mixture was concentrated, and the residue was purified by column chromatography (SiO₂, EtOAc/pet. ether) to obtain 4-oxaspiro[2.4]heptan-6-ol (Intermediate 7-2) (1.0 g, 54% yield) as colorless oil. GC/MS m/z 114.1 [M]⁺.

Step 3: To a stirred solution of Intermediate 7-2 was added sodium hydride (63% dispersion in oil, 1.05 g, 26.3 mmol) at 0° C. After 30 min, a solution of 4-fluoro-3-nitrobenzenesulfonamide (1.92 g, 8.76 mmol) in THF (5 mL) was added dropwise at 0° C. The reaction was warmed to rt and stirred for 6 h. The reaction was cooled to 0° C. and quenched with sat. aq. NH₄Cl and extracted with EtOAc (3×50 mL). The combined organic layers were dried over Na₂SO₄ and concentrated. The residue was triturated with Et₂O and n-pentane to afford Intermediate 7 (700 mg, 25% yield) as a white solid. LC/MS (ESI) m/z 313.0 [M−H]⁻.

### Intermediate 8

### 4-(2-(2-Oxa-6-azaspiro[3.3]heptan-6-yl)ethoxy)-3-nitrobenzenesulfonamide

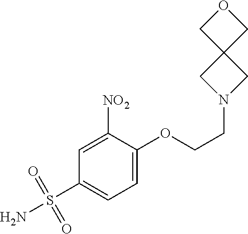 [SMILES: NS(=O)(=O)c1ccc(OCCN2CC3(COC3)C2)c([N+](=O)[O-])c1]

Intermediate 8 was prepared following the procedure described in Step 3 for Intermediate 7 by using 2-(2-oxa-6-azaspiro[3.3]heptan-6-yl)ethanol in place of Intermediate 7-2. LC/MS (ESI) m/z 344.2 [M+H]⁺.

### Intermediate 9

### 4-(2-oxaspiro[3.3]heptan-6-ylmethoxy)-3-nitrobenzenesulfonamide

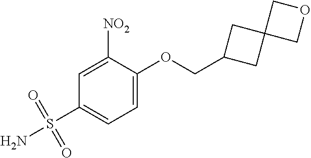 [SMILES: NS(=O)(=O)c1ccc(OCC2CC3(COC3)C2)c([N+](=O)[O-])c1]

Intermediate 9 was prepared following the procedure described in Step 3 for the synthesis of Intermediate 7 by using 2-oxaspiro[3.3]heptan-6-ylmethanol in place of Intermediate 7-2. LC/MS (ESI) m/z 327.4 [M−H]⁻.

### Intermediate 10

### N-methoxy-N,3-dimethylbicyclo[1.1.1]pentane-1-carboxamide

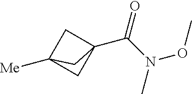

To a stirred solution of 3-methylbicyclo[1.1.1]pentane-1-carboxylic acid (3 g, 23.8 mmol) in DCM (100 mL) was added N,O-dimethylhydroxylamine hydrochloride (3.48 g, 35.7 mmol) and Et₃N (11.6 ml, 83.2 mmol) at rt. The mixture was then cooled to 0° C. and T₃P (50 wt. % in EtOAc, 6.43 g, 40.4 mmol) was added dropwise and reaction was warmed to rt. After 16 h, the reaction was quenched with water (100 mL) and extracted with DCM (3×100 mL). The combined organic layers were dried over Na₂SO₄, filtered and concentrated. The residue was purified by chromatography (SiO2, EtOAc/pet. ether) to provide Intermediate 10 as an oil (2.5 g, 62% yield). ¹H NMR (300 MHz, CDCl₃) δ 3.65 (s, 3H), 3.17 (s, 3H), 1.98 (s, 6H), 1.18 (s, 3H).

### Intermediate 11

### 3-Fluoro-N-methoxy-N-methylbicyclo[11.1.1]pentane-1-carboxamide

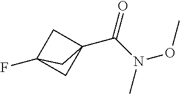

Intermediate 11 was prepared following the procedure described for the synthesis of Intermediate 10 by using 3-fluorobicyclo[1.1.1]pentane-1-carboxylic acid in place of 3-methylbicyclo[1.1.1]pentane-1-carboxylic acid. LC/MS (ESI) m/z 174.3 [M+H]⁺.

### Intermediate 12

### 3-isopropyl-N-methoxy-N-methylbicyclo[1.1.1]pentane-1-carboxamide

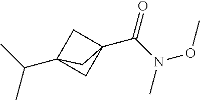

Intermediate 12 was prepared following the procedure described for the synthesis of Intermediate 10 by using 3-isopropylbicyclo[1.1.1]pentane-1-carboxylic acid in place of 3-methylbicyclo[1.1.1]pentane-1-carboxylic acid. LC/MS (ESI) m/z 198.4 [M+H]⁺.

### Intermediate 13

### 3-(1,1-Difluoroethyl)-N-methoxy-N-methylbicyclo[11.1.1]pentane-1-carboxamide

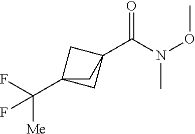

Step 1: To a stirred solution of 3-(methoxycarbonyl)bicyclo[1.1.1]pentane-1-carboxylic acid (10 g, 58.8 mmol), N,O-dimethylhydroxylamine hydrochloride (6.88 g, 42.4 mmol) and triethylamine (12.3 mL, 176.4 mmol) in DCM (200 mL) at 0° C. was added T₃P (50% solution in EtOAc, 18.8 g, 58.8 mmol). The resulting reaction mixture warmed to rt and stirred for 16 h. The reaction mixture was quenched with water (250 mL) and extracted with DCM (3×250 mL). The combined organic layers were dried over Na₂SO₄ and concentrated. The crude product was purified by column chromatography (SiO₂, EtOAc/pet. ether) to provide methyl-3-(methoxy(methyl)carbamoyl)bicyclo[1.1.1]pentane-1-carboxylate (Intermediate 13-1) (9.5 g, 76% yield) as a colorless oil. ¹H NMR (400 MHz, CDCl₃) δ 3.69 (s, 3H), 3.68 (s, 3H), 3.19 (s, 3H), 2.38 (s, 6H).

Step 2: To a stirred solution of Intermediate 13-1 (5 g, 23.5 mmol) in THF (100 mL) at −78° C. was added MeMgBr (3M in Et₂O, 31.3 mL, 93.8 mmol). After stirring for 2 h at −78° C., the reaction was quenched with sat. aq. NH₄Cl (100 mL) and extracted with EtOAc (3×100 mL). The combined organic layers were dried over Na₂SO₄, filtered and concentrated. The crude product was purified by column chromatography (SiO₂, EtOAc/pet. ether) to provide methyl-3-acetylbicyclo[1.1.1]pentane-1-carboxylate (Intermediate 13-2) (2 g, 51% yield) as a white solid. ¹H NMR (400 MHz, CDCl₃) δ 3.70 (s, 3H), 2.29 (s, 6H), 2.14 (s, 3H).

Step 3: A solution of the Intermediate 13-2 (2.3 g, 13.6 mmol) in DCM (50 mL) at −78° C. was treated dropwise with DAST (6.62 g, 41.0 mmol). After the addition, the temperature was raised to rt. After 16 h, the reaction mixture was cooled to −78° C. and carefully quenched with sat. aq. NaHCO₃ (100 mL). The mixture was extracted with DCM (3×100 mL) and the combined organic layers were dried over Na₂SO₄, filtered and concentrated. The crude product was purified by chromatography chromatography (SiO₂, EtOAc/pet. ether) to provide methyl-3-(1,1-difluoroethyl)bicyclo[1.1.1]pentane-1-carboxylate (Intermediate 13-3) (1.8 g, 69% yield) as a clear oil. ¹H NMR (400 MHz, CDCl₃) δ 3.70 (s, 3H), 2.12 (s, 6H), 1.55 (t, J=18.0 Hz, 3H).

Step 4: To a stirred solution of Intermediate 13-3 (1.8 g, 9.46 mmol) and N,O-dimethylhydroxylamine hydrochloride (0.923 g, 9.46 mmol) in anhydrous THF (40 mL) at −78° C. was added i-PrMgCl (2M in THF, 18.9 mL, 37.8 mmol). The reaction mixture was warmed −50° C. and stirred for 2 h. The reaction mixture was quenched with sat. aq. NH₄Cl (50 mL) and extracted with EtOAc (3×75 mL). The combined organic layers were dried over Na₂SO₄, filtered and concentrated. The crude product was purified by column chromatography (SiO₂, EtOAc/pet. ether) to provide Intermediate 13 (1.7 g, 82% yield) as a clear oil. LC/MS (ESI) m/z 220.4 [M+H]⁺.

### Intermediate 14

### 4-[[((1-Methyl-4-piperidinyl)methyl]amino]-3-nitrobenzenesulfonamide

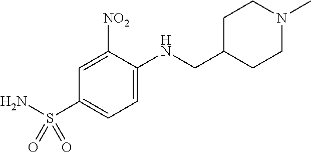 [SMILES: CN1CCC(CNc2ccc(S(N)(=O)=O)cc2[N+](=O)[O-])CC1]

To a solution of (1-methylpiperidin-4-yl)methanamine (1 g, 7.80 mmol) in THF (75 mL), was added 4-fluoro-3-nitrobenzenesulfonamide (1.71 g, 7.80 mmol) followed by triethylamine (3.15 g, 31.2 mmol) and the reaction was stirred at rt. After 16 h, the reaction was concentrated, diluted with water (50 mL) and extracted with 10% MeOH in DCM (3×50 mL). The combined organic layers were dried over Na₂SO₄, filtered and concentrated. The crude product was purified by column chromatography (C18, 0.1% HCO₂H(aq)/MeCN) to obtain 650 mg of 4-((1-methylpiperidin-4-yl)methylamino)-3-nitrobenzenesulfonamide as the formate salt. The compound was dissolved in 10% MeOH in DCM (50 mL) and washed with sat. aq. NaHCO₃. The organic layer was dried over Na₂SO₄, filtered and concentrated to afford Intermediate 14 as a yellow solid (510 mg, 20% yield). LC/MS (ESI) m/z 329.2 [M+H]⁺.

### Intermediate 15

### 4-(((4-fluoro-1-methylpiperidin-4-yl)methyl)amino)-3-nitrobenzenesulfonamide

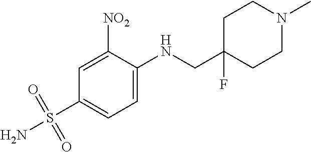 [SMILES: CN1CCC(F)(CNc2ccc(S(N)(=O)=O)cc2[N+](=O)[O-])CC1]

Step 1: To a stirred solution of tert-butyl 4-(aminomethyl)-4-fluoropiperidine-1-carboxylate (2.00 g, 8.61 mmol) in THF (30 mL), was added 4-fluoro-3-nitrobenzenesulfonamide (2.08 g, 9.47 mmol) followed by triethylamine (4.8 mL, 34.45 mmol). The resulting reaction mixture was stirred at rt for 16 h. The reaction was then concentrated, and the resulting residue was diluted with 10% MeOH-DCM (50 mL) and washed with ice-cold water (5×50 mL). The organic layer was dried over Na₂SO₄, filtered and concentrated. The crude product was purified by trituration with Et₂O to afford tert-butyl 4-fluoro-4-(((2-nitro-4-sulfamoylphenyl)amino)methyl)piperidine-1-carboxylate (Intermediate 15-1) (1.6 g, 43% yield). LC/MS (ESI) m/z 333.10 [M-C₅H₉O₂+H]⁺.

Step 2: To a stirred solution of Intermediate 15-1 (1.6 g, 3.70 mmol) in 1,4-dioxane (10 mL) at 0° C. was added HCl (4M HCl in 1,4-dioxane, 20 mL). The reaction was warmed to rt and stirred for 6 h. The reaction was concentrated and triturated with Et₂O to afford 4-(((4-fluoropiperidin-4-yl)methyl)amino)-3-nitrobenzenesulfonamide hydrochloride (Intermediate 15-2) (1.3 g, 96%) as a yellow solid. LC/MS (ESI) m/z 333.1 [C₁₂H₁₇FN₄O₄S+H]⁺.

Step 3: To a stirred solution of Intermediate 15-2 (430 mg, 1.35 mmol) in MeOH (15 mL) was added paraformaldehyde (81 mg, 2.71 mmol) at 0° C. After 15 min, NaCNBH₃ (128 mg, 2.03 mmol) was added and the reaction was warmed to rt. After 18 h, the reaction was quenched sat. aq. NaHCO₃ (15 mL) and the reaction was extracted with DCM (3×100 mL). The combined organic layers were dried over Na₂SO₄, filtered and concentrated. The crude product was triturated with Et₂O followed by 1:1 EtOAc/Hexane to afford Intermediate 15 (340 mg, 25% yield) as a yellow solid. LC/MS (ESI) m/z 347.1 [M+H]⁺.

### Intermediate 16

### 4-(((1r,4r)-4-(dimethylamino)cyclohexyl)amino)-3-nitrobenzenesulfonamide

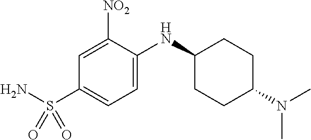 [SMILES: CN(C)[C@H]1CC[C@H](Nc2ccc(S(N)(=O)=O)cc2[N+](=O)[O-])CC1]

To a stirred solution of trans-N,N-dimethylcyclohexane-1,4-diamine dihydrochloride (350 mg, 1.39 mmol) in THF (10 mL) was added 4-fluoro-3-nitrobenzenesulfonamide (322 mg, 1.39 mmol) followed by triethylamine (844 mg, 8.34 mmol). After stirring for 16 h at rt, the reaction was concentrated and triturated with EtOAc and Et₂O to provide the crude product. The product was further purified by HPLC (75:25 to 1:99 10 mM NH₄OAc(aq):CH₃CN) to provide Intermediate 16 as a yellow solid. LC/MS (ESI) m/z 343.1 [M+H]⁺.

### Intermediate 17

### 4-((4-Methylmorpholin-2-yl)methylamino)-3-nitrobenzenesulfonamide

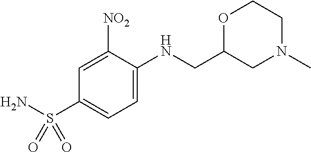 [SMILES: CN1CCOC(CNc2ccc(S(N)(=O)=O)cc2[N+](=O)[O-])C1]

To a stirred solution of (4-mehymorpholin-2-yl)methanamine (400 mg, 3.07 mmol) in THF (25 mL) was added 4-fluoro-3-nitrobenzenesulfonamide (609 mg, 2.76 mmol) followed by triethylamine (1.24 g, 12.28 mmol). After stirring at rt for 16 h, the reaction was concentrated and the resulting crude was diluted with 10% MeOH-DCM (50 mL), and washed with ice-cold water (3×50 mL). The organic layer was dried over Na₂SO₄, filtered and concentrated. The crude product was triturated with Et₂O/pentane to afford Intermediate 17 (600 mg, 65% yield) as a yellow solid. LC/MS (ESI) m/z 331.2 [M+H]⁺.

### Intermediate 17A

### (R)-4-(((4-methylmorpholin-2-yl)methyl)amino)-3-nitrobenzenesulfonamide

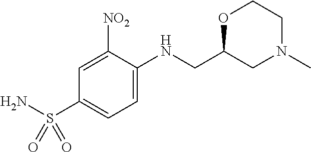 [SMILES: CN1CCO[C@H](CNc2ccc(S(N)(=O)=O)cc2[N+](=O)[O-])C1]

Racemic 4-((4-Methylmorpholin-2-yl)methylamino)-3-nitrobenzenesulfonamide (400 mg) was subjected to chiral SFC separation (Chiralpak AD-H (250×30 mm), 5μ, 30% MeOH) to afford 4-((4-Methylmorpholin-2-yl)methylamino)-3-nitrobenzenesulfonamide (160 mg) as the first eluted peak (RT=3.06 min) with 99.6% ee. LC/MS (ESI) m/z 331.2 [M+H]⁺. The absolute stereochemistry was arbitrarily assigned for Intermediate 17A.

### Intermediate 17B

### (S)-4-(((4-methylmorpholin-2-yl)methyl)amino)-3-nitrobenzenesulfonamide

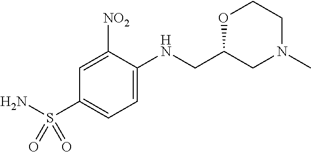 [SMILES: CN1CCO[C@@H](CNc2ccc(S(N)(=O)=O)cc2[N+](=O)[O-])C1]

Racemic 4-((4-Methylmorpholin-2-yl)methylamino)-3-nitrobenzenesulfonamide (400 mg) was subjected to chiral SFC separation (Chiralpak AD-H (250×30 mm), 5μ, 30% MeOH) to afford 4-((4-Methylmorpholin-2-yl)methylamino)-3-nitrobenzenesulfonamide (150 mg) as the second eluted peak (RT=3.64 min) with 99.8% ee. LC/MS (ESI) m/z 331.2 [M+H]⁺. The absolute stereochemistry was arbitrarily assigned for Intermediate 17B.

### Intermediate 18

### 4-((((1r,4r)-4-hydroxy-4-methylcyclohexyl)methyl)amino)-3-nitrobenzenesulfonamide

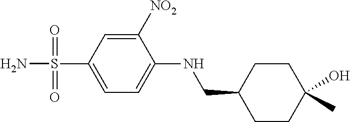 [SMILES: C[C@]1(O)CC[C@H](CNc2ccc(S(N)(=O)=O)cc2[N+](=O)[O-])CC1]

Intermediate 18 was prepared following a procedure described in WO2014/165044A1. LC/MS (ESI) m/z 344.1 [M+H]⁺.

### Intermediate 19

### 2-(Diethoxymethyl)-5,5-dimethylcyclohexan-1-one

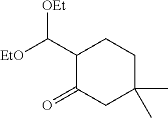 [SMILES: CCOC(OCC)C1CCC(C)(C)CC1=O]

To a solution of triethyl orthoformate (1.32 L, 7.923 mol) in DCM (8.0 L) at −30° C. was added BF₃.OEt₂ (1.244 L, 9.9 mmol) dropwise over 30 min. The reaction mixture was warmed to 0° C. and stirred for 30 min. The reaction mixture was then cooled to −78° C. and 3,3-dimethylcyclohexanone (500 g, 3.96 mol) and N,N-diisopropylethylamine (2.08 L, 11.9 mol) were added dropwise and the reaction was stirred for 2 h at the same temperature. The reaction was then carefully poured into a mixture of sat. aq. NaHCO₃ (25 L) and DCM (10 L). The resulting mixture was stirred for 15 min at rt and the organic layer was separated. The aqueous layer was extracted with DCM (2×10 L) and the combined organic layers were washed with 10% NaCl(aq.) (5 L), dried over Na₂SO₄, filtered and concentrated. The crude product was purified by column chromatography (SiO₂, EtOAc/pet. ether) to afford Intermediate 19 (750 g, 83% yield) as a pale yellow oil. ¹H NMR (400 MHz, CDCl₃) δ 4.83 (d, J=6.0 Hz, 1H), 3.73-3.57 (m, 4H), 2.56-2.53 (m, 1H), 2.20-2.14 (m, 2H), 2.11-2.10 (m, 1H), 1.81 (m, 1H), 1.62-1.56 (m, 2H), 1.21-1.17 (m, 6H), 1.01 (s, 3H), 0.91 (s, 3H).

### Intermediate 20

### Benzyl 2-bromo-4,4-dimethylcyclohex-1-ene-1-carboxylate

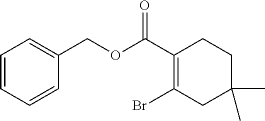 [SMILES: CC1(C)CCC(C(=O)OCc2ccccc2)=C(Br)C1]

Step 1: A solution of NaClO₂ (11.08 g, 122.5 mmol) in water (100 mL) was added drop wise to a stirring mixture of 2-bromo-4,4-dimethylcyclohex-1-ene-1-carbaldehyde (19 g, 87.5 mmol), CH₃CN (100 mL), NaH₂PO₄ (2.72 g, 22.75 mmol), water (40 mL) and 30% H₂O₂(aq.) (15 mL) at 10° C. Upon completion, the reaction, was poured into sat. aq. Na₂CO₃ (200 mL) and washed with Et₂O (200 mL). The aqueous phase was poured into 1N HCl solution (500 mL) and extracted with Et₂O (3×200 mL). The combined organic layers were dried over Na₂SO₄, filtered and concentrated. The crude compound was further washed with water and dried to obtain 2-bromo-4,4-dimethylcyclohex-1-ene-1-carboxylic acid (Intermediate 20-1) (15 g, 73% yield) as a white solid. LC/MS (ESI) m/z 231.0 [M−H]⁻.

Step 2: To a stirred solution of Intermediate 20-1 (10 g, 42.9 mmol) in DMF (100 mL) was added K₂CO₃ (17.79 g, 128.7 mmol) followed by benzyl bromide (14.67 g, 85.8 mmol) at 0° C. and the reaction was warmed to rt. After 16 h, water (200 mL) was added and the reaction was extracted with EtOAc (3×200 mL). The combined organic layers were washed with water (3×200 mL), dried over Na₂SO₄, filtered and concentrated. The crude product was purified by column chromatography (SiO₂, EtOAc/pet. ether) to afford Intermediate 20 (11 g, 79% yield) as a colorless oil. ¹H NMR (400 MHz, CDCl₃) δ 7.43-7.32 (m, 5H), 5.22 (s, 2H), 2.45-2.38 (m, 4H), 1.44 (t, J=5.6 Hz, 2H), 0.97 (s, 6H); GC/MS m/z 322.1 [M]⁺.

### Intermediate 21

### 3-(difluoromethyl)-N-methoxy-N-methylbicyclo[1.1.1]pentane-1-carboxamide

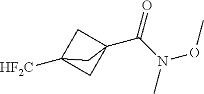

Step 1: A stirring solution of methyl 3-formylbicyclo[1.1.1]pentane-1-carboxylate (7.5 g, 48.7 mmol) in DCM (100 mL) was cooled to −78° C., and treated with DAST (19.3 mL, 146.1 mmol) drop wise and warmed to rt. After 6 h, the reaction mixture was cooled to −78° C. and quenched with sat. aq. NaHCO₃ (100 mL) and extracted with DCM (3×100 mL). The combined organic layers were dried over Na₂SO₄, filtered and concentrated to afford methyl 3-(difluoromethyl) bicyclo[1.1.1] pentane-1-carboxylate (Intermediate 21-1) (7 g) as a viscous oil. This was used in the next step without further purification. ¹H NMR (300 MHz, CDCl₃) δ 5.71 (t, J=56.1 Hz, 1H), 3.70 (s, 3H), 2.15 (s, 6H).

Step 2: To a stirred solution of Intermediate 21-1 (7 g, 39.74 mmol) in anhydrous THF (70 mL) was added N,O-dimethylhydroxylamine hydrochloride (3.89 g, 39.74 mmol) at −78° C., followed by i-PrMgCl (2M in THF, 79.5 mL, 159 mmol). The reaction was warmed to −50° C. and stirred for 2 h. The reaction mixture was then quenched with sat. aq. NH₄Cl solution (100 mL) and extracted with EtOAc (3×100 mL). The combined organic layers were dried over Na₂SO₄, filtered and concentrated. The crude product was purified by column chromatography (SiO₂, EtOAc/pet. ether) to afford Intermediate 21 (4 g, 40% yield over two steps). ¹H NMR (400 MHz, CDCl₃) δ 5.72 (t, J=56.4 Hz, 1H), 3.68 (s, 3H), 3.19 (s, 3H), 2.20 (s, 6H); LC/MS (ESI) m/z 206.1 [M+H]⁺.

### Intermediate 22

### 4,4-dimethyl-2-(3-methylbicyclo[1.1.1]pentan-1-yl)cyclohex-1-ene-1-carbaldehyde

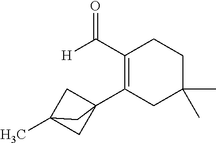

Step 1: A solution of 1-iodo-3-methylbicyclo[1.1.1]pentane (30 g, 144.20 mmol) in THF (225 mL) was cooled to −78° C. and sec-butyllithium (1.4M in cyclohexane, 154.50 mL, 216.30 mmol) was added drop wise over 1 h. The resulting pale yellow suspension was stirred at −78° C. for 10 min and then warmed to 0° C. and stirred for 80 min. The reaction mixture was then cooled to −78° C., and a solution of Intermediate 19 (24.67 g, 108.15 mmol) in THF (75 mL) was added drop wise over 20 min. After 10 min, the reaction was warmed to 0° C. for 1 h. The reaction mixture was then quenched with sat. aq. NH₄Cl (300 mL) and extracted with Et₂O (2×450 mL). The combined organic layers were dried over Na₂SO₄, filtered and concentrated to afford 2-(diethoxymethyl)-5,5-dimethyl-1-(3-methylbicyclo[1.1.1]pentan-1-yl)cyclohexan-1-ol (Intermediate 22-1) (31 g, crude) as a pale yellow oil. This was used in the next step without further purification.

Step 2: A solution of Intermediate 22-1 (62 g, 199.69 mmol) in 1,4-dioxane (1.24 L), was treated with 2N HCl(aq.) (299.5 mL, 599.2 mmol) at rt and then warmed to 70° C. After 16 h, the reaction was cooled to rt, poured into water (1.24 L) and extracted with Et₂O (2×750 mL). The combined organic layers were dried over Na₂SO₄, filtered and concentrated. The crude product was purified by column chromatography (SiO₂, EtOAc/pet. ether) to provide Intermediate 22 (23 g, 36% yield over 2 steps) as a yellow oil. ¹H NMR (400 MHz, CDCl₃): δ 10.28 (s, 1H), 2.25-2.22 (m, 2H), 1.94 (s, 6H), 1.92 (br s, 2H), 1.35-1.32 (m, 2H), 1.19 (s, 3H), 0.90 (s, 6H).

### Intermediate 23

### 2-(3-ethylbicyclo[1.1.1]pentan-1-yl)-4,4-dimethylcyclohex-1-ene-1-carbaldehyde

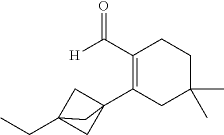 [SMILES: CCC12CC(C3=C(C=O)CCC(C)(C)C3)(C1)C2]

Step 1: To a stirred solution of [1.1.1]propellane (0.19M in Et₂O/pentane), 128.6 mmol) at −78° C. was added EtI (18.7 g, 257.38 mmol). The reaction was warmed to rt and stirred for 3 days in the dark. The reaction was then concentrated at 0° C. to afford 1-ethyl-3-iodobicyclo[1.1.1]pentane (Intermediate 23-1) (21.2 g, 74% yield) as yellow oil. ¹H NMR (400 MHz, CDCl₃) δ 2.17 (s, 6H), 1.52 (q, J=8.0 Hz, 2H), 0.84 (t, J=7.2 Hz, 3H).

Step 2: To a stirred solution of Intermediate 23-1 (10.90 g, 49.1 mmol) in Et₂O (75 mL) at −78° C. was added sec-BuLi (1.4 M in cyclohexane, 50 mL, 70.0 mmol). After 10 min, the reaction was warmed to rt and stirred for 1 h. The reaction mixture was then cooled to −78° C. and treated with a solution of 2-(diethoxymethyl)-5,5-dimethylcyclohexan-1-one (8 g, 35.0 mmol) in Et₂O (25 mL). After 1 h, the reaction was warmed to 0° C. and stirred for 2 h.

The reaction was quenched with sat. aq. NH₄Cl (20 mL) and extracted with EtOAc (3×70 mL). The combined organic layers were then dried over Na₂SO₄, filtered and concentrated to provide 8.5 g of crude 2-(diethoxymethyl)-1-(3-ethylbicyclo[1.1.1]pentan-1-yl)-5,5-dimethylcyclohexan-1-ol (Intermediate 23-2). This was used in the next step without further purification.

Step 3: A solution of Intermediate 23-2 (8.5 g, crude) in acetone (80 mL), was treated with 2N HCl(aq.) (20 mL) at rt and then warmed to 75° C. After 24 h, the reaction was concentrated and then diluted with water (50 mL) and extracted with Et₂O (3×250 mL). The combined organic layers were washed with sat. aq. NaHCO₃, dried over Na₂SO₄ and concentrated. The crude product was purified by column chromatography (SiO₂, Et₂O/pet. ether) to provide Intermediate 23 (3.9 g, 48% yield over 2 steps) as a brown oil. ¹H NMR (400 MHz, CDCl₃) δ 10.30 (s, 1H), 2.26-2.22 (m, 2H), 1.93-1.92 (m, 2H), 1.89 (s, 6H), 1.49 (q, J=7.2 Hz, 2H), 1.33 (t, J=6.4 Hz, 2H), 0.89 (s, 6H), 0.87 (t, J=7.6 Hz, 3H).

### Intermediate 24

### 2-(3-(difluoromethyl)bicyclo[1.1.1]pentan-1-yl)-4,4-di ethylcyclohex-1-ene-1-carbaldehyde

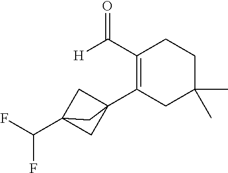 [SMILES: CC1(C)CCC(C=O)=C(C23CC(C(F)F)(C2)C3)C1]

Step 1: Preparation of CF₂HI (based on a procedure from Cao, P. et. al. J. Chem. Soc., Chem. Commun. 1994, 737-738): performed in two parallel batches: A mixture of KI (94 g, 568 mol), MeCN (228 ml) and water (18 mL) was heated to 45° C. and treated with, 2,2-difluoro-2-(fluorosulfonyl)acetic acid (50 g, 284 mmol) in MeCN (50 mL) dropwise over 4 h. The reaction mixture was then cooled to 0° C., and diluted with pentane (150 mL) and water (125 mL). The aqueous layer was washed with pentane (150 mL), and the combined organic layers from both reactions were washed with sat. aq. NaHCO₃ (200 mL), and dried over Na₂SO₄ to obtain 500 mL of difluoromethyl iodide solution. The solution was washed with additional water (2×200 mL) to remove residual acetonitrile, and dried over Na₂SO₄ to obtain difluoroiodomethane (Intermediate 24-1) (0.15 M in pentane, 400 mL, 11% yield). ¹H NMR (400 MHz, CDCl₃) δ 7.67 (t, J=56.0 Hz, 1H).

Step 2: To a stirred solution of [1.1.1]propellane (0.53 M in Et₂O, 52 mL, 27.56 mmol) at −40° C. was added Intermediate 24-1 (0.15 M in pentane, 200 mL, 30 mmol). The reaction mixture was warmed to rt, protected from light, and stirred for 2 days. The reaction was then concentrated at 0-10° C. to obtain 1-(difluoromethyl)-3-iodobicyclo[1.1.1]pentane (Intermediate 24-2) (5 g, 20.5 mmol, 74% yield) as a white solid. ¹H NMR (400 MHz, CDCl₃) δ 5.65 (t, J=56.0 Hz, 1H), 2.40 (s, 6H).

Step 3: A solution of Intermediate 24-2 (30 g, 122.94 mmol) in THF (225 mL) was cooled to −78° C. and sec-butyllithium (1.4M in cyclohexane, 219 mL, 306.7 mmol) was added drop-wise for 1 h. The resulting pale yellow suspension was stirred at −78° C. for 10 min and temperature was raised to 0° C. and stirred for 80 min. The reaction mixture was then cooled to −78° C., and a solution of Intermediate 19 (21 g, 92.20 mmol) in THF (75 mL) was added drop wise to the reaction over 20 min. After 10 min, the reaction was warmed to 0° C. for 1 h. The reaction mixture was quenched with sat. aq. NH₄Cl (450 mL) and extracted with Et₂O (2×300 mL). The combined organic layers were dried over Na₂SO₄, filtered and concentrated to afford 2-(diethoxymethyl)-1-(3-(difluoromethyl)bicyclo[1.1.1]pentan-1-yl)-5,5-dimethylcyclohexan-1-ol (Intermediate 24-3) (31 g, crude) as pale yellow oil. The crude product was used in the next step without further purification.

Step 4: Intermediate 24 was prepared following the procedure described in Step 2 for Intermediate 22 using Intermediate 24-3 in place of Intermediate 22-1 (38% over 2 steps). ¹H NMR (400 MHz, CDCl₃): δ 10.26 (s, 1H), 5.73 (t, J=56.0 Hz, 1H), 2.29-2.25 (m, 2H), 2.18 (s, 6H), 1.94-1.93 (m, 2H), 1.37 (t, J=6.8 Hz, 2H), 0.91 (s, 6H).

### Intermediate 25

### 4,4-dimethyl-2-(3-(trifluoromethyl)bicyclo[1.1.1]pentan-1-yl)cyclohex-1-ene-1

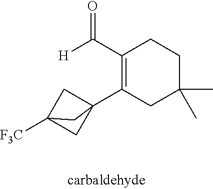 [SMILES: CC1(C)CCC(C=O)=C(C23CC(C(F)(F)F)(C2)C3)C1]

Step 1: To a stirred solution of 1-iodo-3-(trifluoromethyl)bicyclo[1.1.1]pentane (5.00 g, 19.1 mmol) in Et₂O (100 mL) at −78° C. was added sec-BuLi (1.4 M in cyclohexane, 13.63 mL, 19.08 mmol. After 10 minutes at −78° C., the reaction was warmed to 0° C. and stirred for 1 h. The reaction mixture was then cooled to −78° C. and then a solution of Intermediate 19 (3.63 g, 15.90 mmol) in Et₂O (50 mL) was added. After 1 h, the reaction was warmed to 0° C. and stirred for 2 h and then warmed to rt for 1 h. The reaction mixture was quenched with sat. aq. NH₄Cl (100 mL) and extracted with Et₂O (3×150 mL). The organic layers were then dried over Na₂SO₄, filtered and concentrated to provide 2-(diethoxymethyl)-5,5-dimethyl-1-(3-(trifluoromethyl)bicyclo[1.1.1]pentan-1-yl)cyclohexanol (Intermediate 25-1) (7 g, crude) as a brown oil. The crude product was used in the next step without further purification.

Step 2: Intermediate 25 was prepared following the procedure described in Step 3 for Intermediate 23 using Intermediate 25-1 in place of Intermediate 23-2. ¹H NMR (400 MHz, CDCl₃) δ 10.23 (s, 1H), 2.29 (s, 6H), 2.28-2.26 (m, 2H), 1.92 (t, J=2.0 Hz, 2H), 1.36 (t, J=6.8 Hz, 2H), 0.91 (s, 6H).

### Intermediate 26

### 2-((1H-pyrrolo[2,3-b]pyridin-5-yl)oxy)-4-(4-((2-(3-chlorobicyclo[1.1.1]pentan-1-yl)-4,4-dimethylcyclohex-1-en-1-yl)methyl)piperazin-1-yl)benzoic acid

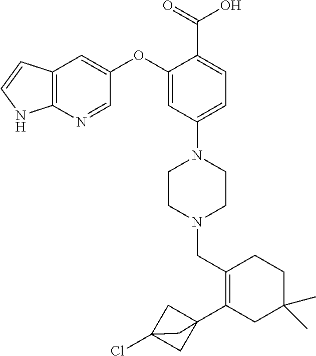 [SMILES: CC1(C)CCC(CN2CCN(c3ccc(C(=O)O)c(Oc4cnc5[nH]ccc5c4)c3)CC2)=C(C23CC(Cl)(C2)C3)C1]

Step 1: A solution of 5-iodo-4,4-dimethylpent-1-ene (9.85 g, 44.0 mmol) in pentane (100 mL) was treated with t-BuLi (64.6 mL, 1.7 M in n-pentane, 109.9 mmol) at −78° C. under inert atmosphere. After 1 h, a solution of Intermediate 1 (5 g, 26.4 mmol) in THF (20 mL) was added and the mixture was stirred at −78° C. for 1 h. The reaction was then warmed to −30° C. over 30 min. and stirred for 1 h. The reaction was quenched with sat. aq. NH₄Cl at −30° C., warmed to rt and extracted with EtOAc (3×200 mL). The combined organic layers were washed with water, dried over Na₂SO₄, filtered and concentrated. The product was purified by column chromatography (SiO₂, EtOAc/pet. ether) to provide 1-(3-chlorobicyclo[1.1.1] pentan-1-yl)-3,3-dimethylhex-5-en-1-one (Intermediate 26-1) (7 g, 70%) as an oil. ¹H NMR (300 MHz, CDCl₃) δ 5.83-5.69 (m, 1H), 5.05-4.96 (m, 2H), 2.36 (s, 6H), 2.30 (s, 2H), 2.09 (d, J=7.5 Hz, 2H), 0.98 (s, 6H).

Step 2: A solution of Intermediate 26-1 (3.1 g, 13.7 mmol) and acrylonitrile (2.18 g, 41.0 mmol) in degassed DCM (120 mL) was treated dropwise over 2 h with a solution of Hoveyda-Grubbs Catalyst™ 2^{nd} Generation (343 mg, 0.55 mmol) in DCM (5 mL) at 45° C. The reaction was stirred at 45° C. for 48 h, cooled to rt, concentrated and absorbed onto Celite. The residue was purified by column chromatography (SiO₂, EtOAc/pet. ether) to afford 7-(3-Chlorobicyclo[1.1.1]pentan-1-yl)-5,5-dimethyl-7-oxohept-2-enenitrile (Intermediate 26-2) as mixture of E/Z isomers (1.3 g, 38%) as a clear colorless oil. LC/MS (ESI) m/z 252.1 [M+H]⁺.

Step 3: A solution of Intermediate 26-2 (700 mg, 2.78 mmol) in MeOH (20 mL) was treated with Pd/C (10 wt %, 170 mg) and stirred under an atmosphere of H₂ (1 atm) for 2 h. The reaction was purged with N2 and the reaction mixture was filtered over Celite and concentrated to provide 7-(3-chlorobicyclo[1.1.1]pentan-1-yl)-5,5-dimethyl-7-oxoheptanenitrile (Intermediate 26-3) (550 mg, 77%) as a clear colorless oil. ¹H NMR (300 MHz, CDCl₃) δ 2.37 (s, 6H), 2.35-2.30 (m, 4H), 1.66-1.55 (m, 2H), 1.52-1.44 (m, 2H), 0.98 (s, 6H).

Step 4: A solution of Intermediate 26-3 (1.1 g, 4.34 mmol, 1 eq) in THF (20 mL) was treated with 4 A molecular sieves (100 mg) and 15-Crown-5 (956 mg, 4.34 mmol) and was placed in a preheated 70° C. oil bath. After 2 min, the reaction was treated with t-BuONa (2.09 g, 21.7 mmol) in a single portion. After 5 h, the reaction was cooled to rt and poured into a stirring solution of sat. aq. NH₄CL. The aqueous phase was washed with DCM (3×25 mL). The combined organic layers were dried over Na₂SO₄, filtered and concentrated. The crude product was purified by column chromatography (SiO₂, EtOAc/pet. ether) to afford 2-(3-chlorobicyclo[1.1.1]pentan-1-yl)-4,4-dimethylcyclohex-1-enecarbonitrile (Intermediate 26-4) (800 mg, 39%) as a clear colorless oil. LC/MS (ESI) m/z 236.3 [M+H]⁺.

Step 5: To a stirred solution of Intermediate 26-4 (400 mg, 1.70 mmol) in anhydrous DCM (20 mL) at −78° C. was added DIBAL-H (2.55 mL, 1M in toluene, 2.55 mmol). The reaction was warmed to rt. After 4 h, the reaction was cooled to 0° C., quenched with 2M HCl(aq.) (40 mL) and warmed to rt. The reaction mixture was diluted with water, and extracted with DCM (2×40 mL) and the combined organic layers were dried over Na₂SO₄, filtered and concentrated to provide 2-(3-chlorobicyclo[1.1.1]pentan-1-yl)-4,4-dimethyl cyclohex-1-enecarbaldehyde (Intermediate 26-5) (400 mg, quantitative). This compound was used directly in the next step without further purification. ¹H NMR (300 MHz, CDCl₃) δ 10.19 (s, 1H), 2.44 (s, 6H), 2.30-2.22 (m, 2H), 1.90 (s, 2H), 1.35 (t, J=6 Hz, 2H), 0.90 (s, 6H).

Step 6: To a stirred solution of Intermediate 26-5 (300 mg, 1.26 mmol) in DCM (10 mL) was added Intermediate 2 (544 mg, 1.38 mmol) and NaBH(OAc)₃ (347 mg, 1.64 mmol) at rt. After 16 h, additional NaBH(OAc)₃ (347 mg, 1.64 mmol) was added. After 48 h, the reaction was quenched with MeOH (0.2 mL) at 0° C., warmed to rt and concentrated. The residue was diluted with DCM and washed with sat. aq. NaHCO₃. The aqueous layer was washed with DCM (3×25 mL) and the combined organic layers were dried over Na₂SO₄, filtered and concentrated. The residue was purified by column chromatography (SiO₂, EtOAc/pet. ether) to afford tert-butyl 2-(1H-pyrrolo[2,3-b]pyridin-5-yl-oxy)-4-(4-((2-(3-chlorobicyclo [1.1.1]pentan-1-yl)-4,4-dimethylcyclohex-1-en-yl)methyl)piperazin-1-yl)benzoate (Intermediate 26-6) (220 mg, 44.6 mmol; 28%) as a white solid. LC/MS (ESI) m/z 617.3 [M+H]⁺.

Step 7: To a solution of Intermediate 26-6 (125 mg, 0.20 mmol) in DCM (2 mL) at 0° C. was added TFA (139 mg, 1.22 mmol). The mixture was warmed to rt and stirred for 3 h and concentrated to provide the TFA salt of 2-(1H-pyrrolo[2,3-b]pyridin-5-yl-oxy)-4-(4-((2-(3-chlorobicyclo[1.1.1]pentan-1-yl)-4,4-dimethylcyclohex-1-en-yl)methyl)piperazin-1-yl)benzoic acid (140 mg, quantitative) as a white solid LC/MS (ESI) m/z 561.3 [C₃₂H₃₇ClN₄O₃+H]⁺.

### Intermediate 27

### 2-((1H-pyrrolo[2,3-b]pyridin-5-yl)oxy)-4-(4-((2-(3-fluorobicyclo[1.1.1]pentan-1-yl)-4,4-dimethylcyclohex-1-en-1-yl)methyl)piperazin-1-yl)benzoic acid

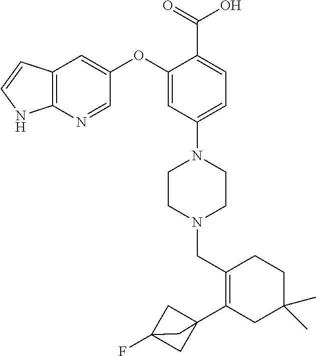 [SMILES: CC1(C)CCC(CN2CCN(c3ccc(C(=O)O)c(Oc4cnc5[nH]ccc5c4)c3)CC2)=C(C23CC(F)(C2)C3)C1]

Step 1: 1-(3-fluorobicyclo[1.1.1]pentan-1-yl)-3,3-dimethylhex-5-en-1-one (Intermediate 27-1) was prepared following the procedure described in Step 1 for Intermediate 26 using Intermediate 11 in place of Intermediate 1. ¹H NMR (300 MHz, CDCl₃) δ 5.84-5.69 (m, 1H), 5.06-4.96 (m, 2H), 2.34 (s, 2H), 2.29 (d, J=2.4 Hz, 6H), 2.10 (d, J=7.2 Hz, 2H), 0.99 (s, 6H).

Step 2: E/Z-7-(3-fluorobicyclo[1.1.1]pentan-1-yl)-5,5-dimethyl-7-oxohept-2-enenitrile (Intermediate 27-2) was prepared following the procedure described in Step 2 for Intermediate 26 using Intermediate 27-1 in place of Intermediate 26-1. LC/MS (ESI) m/z 236.3 [M+H]⁺.

Step 3: 7-(3-fluorobicyclo[1.1.1]pentan-1-yl)-5,5-dimethyl-7-oxoheptanenitrile (Intermediate 27-3) was prepared following the procedure described in Step 3 for Intermediate 26 using Intermediate 27-2 in place of Intermediate 26-2. ¹H NMR (400 MHz, CDCl₃) δ 2.36 (s, 2H), 2.32 (t, J=6.8 Hz, 2H), 2.31 (d, J=2.8 Hz, 6H), 1.64-1.58 (m, 2H), 1.51-1.47 (m, 2H), 0.99 (s, 6H).

Step 4: 2-(3-fluorobicyclo[1.1.1]pentan-1-yl)-4,4-dimethylcyclohex-1-enecarbonitrile (Intermediate 27-4) was prepared following the procedure described in Step 4 for Intermediate 26 using Intermediate 27-3 in place of Intermediate 26-3. LC/MS (ESI) m/z 220.4 [M+H]⁺.

Step 5: 2-(3-fluorobicyclo[1.1.1]pentan-1-yl)-4,4-dimethylcyclohex-1-enecarbaldehyde (Intermediate 27-5) was prepared following the procedure described in Step 5 for Intermediate 26 using Intermediate 27-4 in place of Intermediate 26-4. ¹H NMR (300 MHz, CDCl₃) δ 10.19 (s, 1H), 2.37-2.34 (m, 6H), 2.30-2.25 (m, 2H), 1.93 (br s, 2H), 1.40-1.35 (m, 2H), 0.91 (s, 6H).

Step 6: To a stirred solution of Intermediate 27-5 (100 mg, 0.45 mmol) in EtOH (4 mL) was added Intermediate 2 (195 mg, 0.49 mmol) and AcOH (cat.) at rt and stirred for 15 min. The resulting reaction mixture was cooled to 0° C. and NaCNBH₃ (42 mg, 0.675 mmol) was added and the reaction was warmed to rt. After 16 h, the reaction was concentrated and the residue was diluted with sat. aq. NaHCO₃ (10 ml) and extracted with DCM (3×10 ml). The combined organic layers were dried over Na₂SO₄ and concentrated. The crude compound was purified by column chromatography (SiO₂, EtOAc/pet. ether) to obtain tert-Butyl 2-(1H-pyrrolo[2,3-b]pyridin-5-yl-oxy)-4-(4-((2-(3-fluorobicyclo[1.1.1]pentan-1-yl)-4,4-dimethylcyclohex-1-en-yl)methyl)piperazin-1-yl)benzoate (Intermediate 27-6) as a white solid (40 mg, 15% yield). LC/MS (ESI) m/z 601.7 [M+H]⁺.

Step 7: 2-(1H-pyrrolo[2,3-b]pyridin-5-yl-oxy)-4-(4-((2-(3-fluorobicyclo[1.1.1]pentan-1-yl)-4,4-dimethylcyclohex-1-en-yl)methyl)piperazin-1-yl)benzoic acid as the TFA salt was prepared following the procedure described in Step 7 for Intermediate 26 by reacting Intermediate 27-6 in place of Intermediate 26-6. LC/MS (ESI) m/z 545.4 [C₃₂H₃₇FN₄O₃+H]⁺.

### Intermediate 28

### 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoic acid

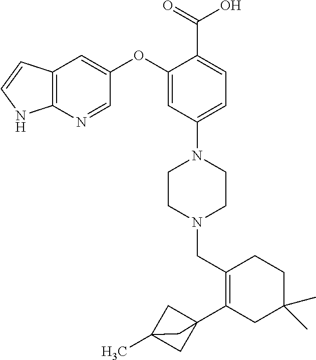 [SMILES: CC1(C)CCC(CN2CCN(c3ccc(C(=O)O)c(Oc4cnc5[nH]ccc5c4)c3)CC2)=C(C23CC(C)(C2)C3)C1]

### Route A:

Step 1: 3,3-dimethyl-1-(3-methylbicyclo[1.1.1]pentan-1-yl)hex-5-en-1-one (Intermediate 28-1) was prepared following the procedure described in Step 1 for Intermediate 26 using Intermediate 10 in place of Intermediate 1. ¹H NMR (300 MHz, CDCl₃) δ 5.86-5.71 (m, 1H), 5.04-4.97 (m, 2H), 2.28 (s, 2H), 2.09 (d, J=7.8 Hz, 2H), 1.85 (s, 6H), 1.12 (s, 3H), 0.97 (s, 6H).

Step 2: E/Z-5,5-dimethyl-7-(3-methylbicyclo[1.1.1]pentan-1-yl)-7-oxohept-2-enenitrile (Intermediate 28-2) was prepared following the procedure described in Step 2 for Intermediate 26 using Intermediate 28-1 in place of Intermediate 26-1. LC/MS (ESI) m/z 232.3 [M+H]⁺.

Step 3: 5,5-dimethyl-7-(3-methylbicyclo[1.1.1]pentan-1-yl)-7-oxoheptanenitrile (Intermediate 28-3) was prepared following the procedure described in Step 3 for Intermediate 26 using Intermediate 28-2 in place of Intermediate 26-2. ¹H NMR (400 MHz, CDCl₃) δ 2.33-2.29 (m, 4H), 1.86 (s, 6H), 1.64-1.56 (m, 2H), 1.50-1.45 (m, 2H), 1.18 (s, 3H), 0.98 (s, 6H).

Step 4: 4,4-dimethyl-2-(3-methylbicyclo[1.1.1]pentan-1-yl)cyclohex-1-enecarbonitrile (Intermediate 28-4) was prepared following the procedure described in Step 4 for Intermediate 26 using Intermediate 28-3 in place of Intermediate 26-3. LC/MS (ESI) m/z 216.4 [M+H]⁺.

Step 5: Intermediate 22 was prepared following the procedure described in Step 5 for Intermediate 26 using Intermediate 28-4 in place of Intermediate 26-4. LC/MS (ESI) m/z 219.3 [M+H]⁺.

Step 6: To a stirred solution of Intermediate 22 (70 mg, 0.32 mmol) in EtOH (4 mL) was added Intermediate 2 (190 mg, 0.48 mmol) and AcOH (cat.) at rt. After 15 min, the mixture was cooled to 0° C., NaCNBH₃ (31 mg, 0.48 mmol) was added and the reaction was warmed to rt. After 16 h, the reaction was concentrated, and the residue was diluted with sat. aq. NaHCO₃ (10 mL) and extracted with DCM (3×10 ml). The combined organic layers were dried over Na₂SO₄, filtered and concentrated. The crude product was purified by column chromatography (SiO₂, EtOAc/pet. ether) to obtain tert-butyl 2-(1H-pyrrolo[2,3-b]pyridin-5-yloxy)-4-(4-((4,4-dimethyl-2-(3-methylbicyclo[1.1.1]pentan-1-yl)cyclohex-1-enyl)methyl)piperazin-1-yl)benzoate (Intermediate 28-5) (80 mg, 42%) as a white solid. LC/MS (ESI) m/z 597.4 [M+H]⁺.

Step 7: 2-(1H-pyrrolo[2,3-b]pyridin-5-yloxy)-4-(4-((4,4-dimethyl-2-(3-methylbicyclo[1.1.1]pentan-1-yl)cyclohex-1-enyl)methyl)piperazin-1-yl)benzoic acid trifluoroacetate was prepared following the procedure described in Step 7 for Intermediate 26 using Intermediate 28-5 in place of Intermediate 26-6. LC/MS (ESI) m/z 541.4 [C₃₃H₄₀N₄O₃+H]⁺.

### Route B:

Step 1: A solution of t-butyl lithium (1.3 M in pentane, 60 mL, 78 mmol) was added dropwise to a solution of 1-iodo-3-methylbicyclo[1.1.1]pentane (6.5 g, 31.2 mmol) in MTBE (60 mL) at −78° C. under N2. The reaction mixture was stirred for 1 h at −78° C. Lithium 2-thienylcyanocuprate (0.25M in THF, 125 mL, 31.2 mmol) was added at −78° C., and the addition was controlled to keep the temperature below −60° C. After the addition, the reaction mixture was warmed to 0° C. and stirred for 30 min. The reaction was then cooled to −78° C. and Intermediate 20 (5 g, 15.5 mmol) in MTBE (5 mL,) was added followed by BF₃.OEt₂ (3.5 mL, 15.5 mmol). The reaction was stirred for 30 min at −78° C. and then warmed to rt. After 16 h, the reaction was cooled to 0° C. and quenched with sat. aq. NH₄Cl (50 mL) and H₂O (50 mL). MTBE (50 mL) was then added and the reaction mixture was stirred for 20 min at rt. The organic layer was separated, and the aqueous layer was extracted with MTBE (100 mL). The combined organic layers were dried over Na₂SO₄, filtered, and concentrated. Purification by column chromatography (SiO₂, EtOAc/Heptane) followed by column chromatography (C18, CH₃CN:H₂O) provided benzyl 4,4-dimethyl-2-(3-methylbicyclo[1.1.1]pentan-1-yl)cyclohex-1-ene-1-carboxylate (3.6 g, 70%). ¹H NMR (400 MHz, DMSO) δ 7.41-7.34 (m, 5H), 5.13 (s, 2H), 2.17-2.12 (m, 2H), 1.72-1.70 (m, 2H), 1.64 (s, 6H), 1.31-1.27 (m, 2H), 1.08 (s, 3H), 0.86 (s, 6H).

Step 2: To a stirred solution of benzyl 4,4-dimethyl-2-(3-methylbicyclo[1.1.1]pentan-1-yl)cyclohex-1-ene-1-carboxylate (1.1 g, 3.39 mmol) in THF (40 mL) at 0° C. was added lithium aluminum hydride (386.6 mg, 10.2 mmol). The reaction was warmed to rt and stirred for 3 h. The reaction was then cooled to 0° C., diluted with Et₂O (40 ml) and treated with H₂O (0.386 mL), 0.386 mL of 15% NaOH(aq.) followed by H₂O (1.15 mL). The reaction was warmed to rt, stirred for 15 min, and then treated with anhydrous MgSO₄. After 15 min, the reaction was filtered, concentrated, and purified by column chromatography (SiO₂, EtOAc/pet. ether) to provide (4,4-dimethyl-2-(3-methylbicyclo[1.1.1]pentan-1-yl)cyclohex-1-en-1-yl)methanol (1.1 g, 68% yield) as a colorless oil. ¹H NMR (400 MHz, CDCl₃) δ 4.15 (d, J=5.2 Hz, 2H), 2.16-2.12 (m, 2H), 1.81 (s, 6H), 1.68 (s, 2H), 1.32 (t, J=6.4 Hz, 2H), 1.15 (s, 3H), 0.86 (s, 6H).

Step 3: To a stirred solution of (4,4-dimethyl-2-(3-methylbicyclo[1.1.1]pentan-1-yl)cyclohex-1-en-1-yl)methanol (500 mg, 2.27 mmol) in DCM (20 mL) at 0° C. was added SOCl₂ (0.537 mL, 4.54 mmol) drop wise. The reaction mixture was warmed to rt and stirred for 2 h. The reaction was concentrated, diluted with DCM and concentrated once more to obtain 1-(2-(chloromethyl)-5,5-dimethylcyclohex-1-en-1-yl)-3-methylbicyclo[1.1.1]pentane (540 mg, quantitative yield) as a clear oil. This was used for the next step without further purification. ¹H NMR (400 MHz, CDCl₃) δ 4.19 (s, 2H), 2.15-2.11 (m, 2H), 1.85 (s, 6H), 1.70 (s, 2H), 1.34 (t, J=6.4 Hz, 2H), 1.16 (s, 3H), 0.87 (s, 6H).

Step 4: To a stirred solution of 1-(2-(chloromethyl)-5,5-dimethylcyclohex-1-en-1-yl)-3-methylbicyclo[1.1.1]pentane (540 mg, 2.26 mmol) in acetone (20 mL) was added methyl 2-((1H-pyrrolo[2,3-b]pyridin-5-yl)oxy)-4-(piperazin-1-yl)benzoate (798 mg, 2.26 mmol), NaI (33.90 mg, 0.22 mmol) and K₂CO₃ (938.9 mg, 6.80 mmol) at rt. The reaction was then heated to reflux for 6 h. The reaction was then cooled to rt, diluted with 50 mL of acetone and filtered. The collected solid was washed with acetone (150 mL) and the combined filtrates were concentrated to provide methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate (1.15 g, 91% yield) as a white solid. LC/MS (ESI) m/z 555.3 [M+H]⁺.

Step 5: To a stirred solution of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate (1.15 g, 2.075 mmol) in MeOH:THF:H₂O (1:1:1) (36 mL) was added LiOH.H₂O (261.30 mg, 6.23 mmol) at rt. The reaction was heated to 30° C. and stirred for 16 h. The volatile solvents were then removed, and the reaction was neutralized with 1N HCl and extracted with DCM (3×70 mL). The combined organic layers were dried over Na₂SO₄, filtered and concentrated to provide Intermediate 28 (940 mg, 84% yield) as a white solid. LC/MS (ESI) m/z 541.3 [M+H]⁺.

### Route C:

Step 1: A solution of methyl 2-((1H-pyrrolo[2,3-b]pyridin-5-yl)oxy)-4-(piperazin-1-yl)benzoate (35 g, 99.3 mmol) and Intermediate 22 (26.0 g, 119.2 mmol) in THF (700 mL) was stirred at rt for 20 min. The reaction was then cooled to 0° C. and NaBH(OAc)₃ (63.15 g, 297.96 mmol) was added. Following the addition, the reaction was warmed to rt. After 16 h, the reaction was poured into ice cold water (1 L), and extracted with EtOAc (2×500 mL). The combined organic layers were washed with 10% NaHCO₃(aq.) (500 mL), and brine (500 mL). The organic layer was then dried over Na₂SO₄, filtered and concentrated. The crude product was first purified by column chromatography (SiO₂, EtOAc/pet. ether) and then triturated with MeOH and filtered to afford methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate as an off white solid (38 g, 70%). LC/MS (ESI) m/z 555.1 [M+H]⁺.

Step 2: Intermediate 28 was prepared following the procedure described in Step 5, Route B for Intermediate 28. LC/MS (ESI) m/z 541.3 [M+H]⁺.

### Intermediate 29

### 2-((1H-pyrrolo[2,3-b]pyridin-5-yl)oxy)-4-(4-((2-(3-ethylbicyclo[1.1.1]pentan-1-yl)-4,4-dimethylcyclohex-1-en-1-yl)methyl)piperazin-1-yl)benzoic acid

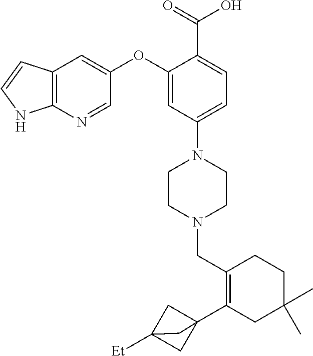 [SMILES: CCC12CC(C3=C(CN4CCN(c5ccc(C(=O)O)c(Oc6cnc7[nH]ccc7c6)c5)CC4)CCC(C)(C)C3)(C1)C2]

Step 1: To a solution of methyl 2-((1H-pyrrolo[2,3-b]pyridin-5-yl)oxy)-4-(piperazin-1-yl)benzoate (1.89 g, 5.38 mmol) in DMSO (25 mL) was added a solution of Intermediate 23 (1.5 g, 6.46 mmol) in THF (25 mL) at rt and the reaction was stirred for 1 h. The reaction was then cooled to 0° C. and treated with Na(OAc)₃BH (3.42 g, 16.14 mmol) and warmed to rt. After 24 h, the reaction was diluted with sat. aq. NaHCO₃, and extracted with 10% MeOH in DCM (4×50 mL). The combined organic layers were dried over Na₂SO₄, filtered and concentrated. The crude product was purified by column chromatography (SiO₂, Et₂O/n-pentane) to afford methyl 2-((1H-pyrrolo[2,3-b]pyridin-5-yl)oxy)-4-(4-((2-(3-ethylbicyclo[1.1.1]pentan-1-yl)-4,4-dimethylcyclohex-1-en-1-yl)methyl)piperazin-1-yl)benzoate (Intermediate 29-1) (1.4 g, 46% yield) as an off white solid. LC/MS (ESI) m/z 569.4 [M+H]⁺.

Step 2: Intermediate 29 was prepared following the procedure described in Step 5, Route B for Intermediate 28 using Intermediate 29-1 in place of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate. LC/MS (ESI) m/z 555.3 [M+H]⁺.

### Intermediate 30

### 2-((1H-pyrrolo[2,3-b]pyridin-5-yl)oxy)-4-(4-((2-(3-(difluoromethyl)bicyclo[1.1.1]pentan-1-yl)-4,4-dimethylcyclohex-1-en-1-yl)methyl)piperazin-1-yl)benzoic acid

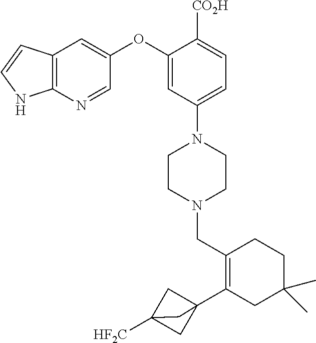

Step 1: Methyl 2-((1H-pyrrolo[2,3-b]pyridin-5-yl)oxy)-4-(4-((2-(3-(difluoromethyl)bicyclo[1.1.1]pentan-1-yl)-4,4-dimethylcyclohex-1-en-1-yl)methyl)piperazin-1-yl)benzoate (Intermediate 30-1) was prepared following the procedure described in Step 1, Route C for Intermediate 28 using Intermediate 24 in place of Intermediate 22. LC/MS (ESI) m/z 591.2 [M+H]⁺.

Step 2: Intermediate 30 was prepared following the procedure described in Step 5, Route B for Intermediate 28 using Intermediate 30-1 in place of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate. LC/MS (ESI) m/z 577.5[M+H]⁺.

### Intermediate 31

### 2-((1H-pyrrolo[2,3-b]pyridin-5-yl)oxy)-4-(4-((4,4-dimethyl-2-(3-(trifluoromethyl)bicyclo[1.1.1]pentan-1-yl)cyclohex-1-en-1-yl)methyl)piperazin-1-yl)benzoic acid

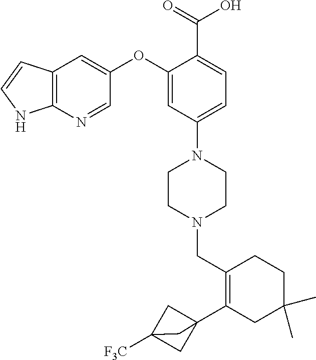 [SMILES: CC1(C)CCC(CN2CCN(c3ccc(C(=O)O)c(Oc4cnc5[nH]ccc5c4)c3)CC2)=C(C23CC(C(F)(F)F)(C2)C3)C1]

Step 1: Representative procedure reaction was performed in 3 parallel batches): To a stirred solution of methyl 2-((1H-pyrrolo[2,3-b]pyridin-5-yl)oxy)-4-(piperazin-1-yl)benzoate (2 g, 5.68 mmol) in DMSO (0.2 M, 30 mL) was added a solution of Intermediate 25 (1.72 g, 6.22 mmol) in THF (30 mL) at rt. After 1 h, the reaction mixture was cooled to 0° C., and treated with NaBH(OAc)₃ (1.70 g, 17.04 mmol). The reaction was warmed to rt and stirred for 24 h. The reaction mixture was diluted with sat. aq. NaHCO₃, and extracted with 10% MeOH in DCM (4×150 mL). The combined organic layers were dried over Na₂SO₄, filtered and concentrated. The crude product was purified by column chromatography (SiO₂ EtOAc/pet. ether) to afford methyl 2-(1H-pyrrolo[2,3-b]pyridin-5-yloxy)-4-(4-((4,4-dimethyl-2-(3-(trifluoromethyl)bicyclo[1.1.1]pentan-1-yl)cyclohex-1-enyl)methyl)piperazin-1-yl)benzoate (Intermediate 31-1) (8.7 g, 14.29 mmol, 84% combined for three batches) as a white solid. LC/MS (ESI) m/z 609.3 [M+H]⁺.

Step 2: To a stirred solution of Intermediate 31-1 (8.3 g, 13.65 mmol) in MeOH:THF:H₂O (1:1:1) (100 mL) was added LiOH.H₂O (1.7 g, 40.95 mmol) at rt. The reaction mixture was then heated to 35° C. and stirred for 16 h. The reaction mixture was concentrated, diluted with water and neutralized with 1N HCl. The product was then extracted with 10% MeOH-DCM (3×150 mL). The combined organic layers were dried over Na₂SO₄, filtered and concentrated to provide Intermediate 31 (7.6 g, 90% yield) as a white solid. ¹H NMR (400 MHz, DMSO-d₆) δ 11.91 (br s, 1H), 11.59 (s, 1H), 7.98 (d, J=2.4 Hz, 1H), 7.70 (d, J=8.8 Hz, 1H), 7.43 (t, J=2.8 Hz, 1H), 7.37 (d, J=2.4 Hz, 1H), 6.73-6.71 (m, 1H), 6.36-6.34 (m, 2H), 3.14-3.05 (m, 4H), 2.94 (s, 2H), 2.40-2.28 (m, 4H), 2.12 (s, 6H), 2.09-1.99 (m, 2H), 1.68 (s, 2H), 1.29-1.19 (m, 2H), 0.84 (s, 6H); ¹⁹F NMR (376 MHz, DMSO-d₆, unreferenced) 6-71.55; LC/MS (ESI) m/z 595.3 [M+H]⁺.

### Intermediate 32

### 2-((1H-pyrrolo[2,3-b]pyridin-5-yl)oxy)-4-(4-((2-(3-isopropylbicyclo[1.1.1]pentan-1-yl)-4,4-dimethylcyclohex-1-en-1-yl)methyl)piperazin-1-yl)benzoic acid

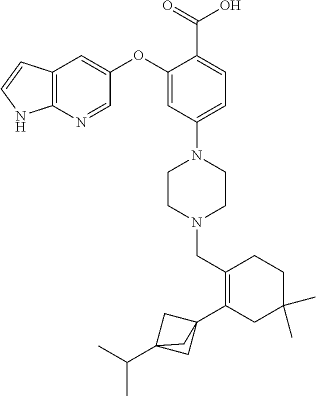 [SMILES: CC(C)C12CC(C3=C(CN4CCN(c5ccc(C(=O)O)c(Oc6cnc7[nH]ccc7c6)c5)CC4)CCC(C)(C)C3)(C1)C2]

Step 1: 3,3-dimethyl-1-(3-isopropylbicyclo[1.1.1]pentan-1-yl)hex-5-en-1-one (Intermediate 32-1) was prepared following the procedure described in Step 1 from Intermediate 26 using Intermediate 12 in place of Intermediate 1. ¹H NMR (400 MHz, CDCl₃) δ 5.81-5.74 (m, 1H), 5.04-4.97 (m, 2H), 2.31 (s, 2H), 2.10 (d, J=7.6 Hz, 2H), 1.76 (s, 6H), 1.69-1.65 (m, 1H), 0.99 (s, 6H), 0.83 (d, J=6.8 Hz, 6H).

Step 2: E/Z-7-(3-isopropylbicyclo[1.1.1]pentan-1-yl)-5,5-dimethyl-7-oxohept-2-enenitrile (Intermediate 32-2) was prepared following the procedure described in Step 2 from Intermediate 26 using Intermediate 32-1 in place of Intermediate 26-1. LC/MS (ESI) m/z 260.4 [M+H]⁺.

Step 3: 7-(3-Isopropylbicyclo[1.1.1]pentan-1-yl)-5,5-dimethyl-7-oxoheptanenitrile (Intermediate 32-3) was prepared following the procedure described in Step 3 from Intermediate 26 using Intermediate 32-2 in place of Intermediate 26-2. ¹HNMR (400 MHz, CDCl₃) δ 2.34-2.30 (m, 4H), 1.78 (s, 6H), 1.70-1.57 (m, 4H), 1.51-1.46 (m, 1H), 0.98 (s, 6H), 0.84 (d, J=7.2 Hz, 6H).

Step 4: 2-(3-Isopropylbicyclo[1.1.1]pentan-1-yl)-4,4-dimethylcyclohex-1-enecarbonitrile (Intermediate 32-4) was prepared following the procedure described in Step 4 from Intermediate 26 using Intermediate 32-3 in place of Intermediate 26-3. LC/MS (ESI) m/z 244.4 [M+H]⁺.

Step 5: 2-(3-Isopropylbicyclo[1.1.1]pentan-1-yl)-4,4-dimethylcyclohex-1-enecarbaldehyde (Intermediate 32-5) was prepared following the procedure described in Step 5 from Intermediate 26 using Intermediate 32-4 in place of Intermediate 26-4. LC/MS (ESI) m/z 247.4 [M+H]⁺.

Step 6: tert-Butyl 2-(1H-pyrrolo[2,3-b]pyridin-5-yloxy)-4-(4-((2-(3-isopropylbicyclo [1.1.1]pentan-1-yl)-4,4-dimethylcyclohex-1-enyl)methyl)piperazin-1-yl)benzoate (Intermediate 32-6) was prepared following the procedure described in Step 6, Route A for Intermediate 28 using Intermediate 32-5 in place of Intermediate 28-5. LC/MS (ESI) m/z 625.7 [M+H]⁺.

Step 7: To a solution of Intermediate 32-6 (160 mg, 0.26 mmol) in DCM (5 mL) at 0° C. was added TFA (176 mg, 1.54 mmol). The mixture was warmed to rt and stirred for 3 h. The reaction was then diluted with sat. aq. NaHCO₃ (10 mL), and extracted with DCM (3×10 mL). The combined organic layers were dried over Na₂SO₄, filtered and concentrated to provide Intermediate 32 as an off-white solid. LC/MS (ESI) m/z 569.6 [M+H]⁺.

### Intermediate 33

### 2-(1H-pyrrolo[2,3-b]pyridin-5-yloxy)-4-(4-((2-(3-(1,1-difluoroethyl)bicyclo[1.1.1]pentan-1-yl)-4,4-dimethylcyclohex-1-enyl)methyl)piperazin-1-yl)benzoic acid

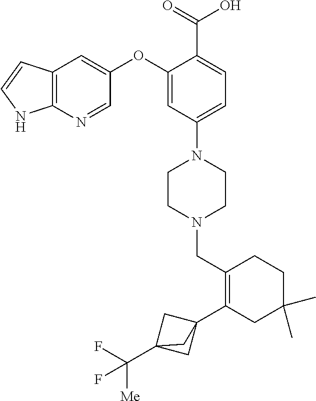 [SMILES: CC1(C)CCC(CN2CCN(c3ccc(C(=O)O)c(Oc4cnc5[nH]ccc5c4)c3)CC2)=C(C23CC(C(C)(F)F)(C2)C3)C1]

Step 1: 1-(3-(1,1-Difluoroethyl)bicyclo[1.1.1]pentan-1-yl)-3,3-dimethylhex-5-en-1-one (Intermediate 33-1) was prepared following the procedure described in Step 1 for Intermediate 26 using Intermediate 13 in place of Intermediate 1. ¹H NMR (400 MHz, CDCl₃) δ 5.85-5.69 (m, 1H), 5.03-4.95 (m, 2H), 2.30 (s, 2H), 2.08 (d, J=8.0 Hz, 2H), 2.03 (s, 6H), 1.53 (t, J=18.0 Hz, 3H), 0.97 (s, 6H).

Step 2: E/Z-7-(3-(1,1-difluoroethyl)bicyclo[1.1.1]pentan-1-yl)-5,5-dimethyl-7-oxohept-2-enenitrile (Intermediate 33-2) was prepared following the procedure described in Step 2 for Intermediate 26 using Intermediate 33-1 in place of Intermediate 26-1. LC/MS (ESI) m/z 282.5 [M+H]⁺.

Step 3: 7-(3-(1,1-Difluoroethyl)bicyclo[1.1.1]pentan-1-yl)-5,5-dimethyl-7-oxoheptanenitrile (Intermediate 33-3) was prepared following the procedure described in Step 3 for Intermediate 26 using Intermediate 33-2 in place of Intermediate 26-2. ¹H NMR (400 MHz, CDCl₃) δ 2.34-2.31 (m, 4H), 2.06 (s, 6H), 1.66-1.57 (m, 2H), 1.55 (t, J=18.0 Hz, 3H), 1.51-1.46 (m, 2H), 0.99 (s, 6H).

Step 4: 2-(3-(1,1-Difluoroethyl)bicyclo[1.1.1]pentan-1-yl)-4,4-dimethylcyclohex-1-enecarbonitrile (Intermediate 33-4) was prepared following the procedure described in Step 4 for Intermediate 26 using Intermediate 33-3 in place of Intermediate 26-3. LC/MS (ESI) m/z 266.1 [M+H]⁺.

Step 5: 2-(3-(1,1-difluoroethyl)bicyclo[1.1.1]pentan-1-yl)-4,4-dimethylcyclohex-1-enecarbaldehyde (Intermediate 33-5) was prepared following the procedure described in Step 5 for Intermediate 26 using Intermediate 33-4 in place of Intermediate 26-4. LC/MS (ESI) m/z 269.5 [M+H]⁺.

Step 6: tert-butyl 2-(1H-pyrrolo[2,3-b]pyridin-5-yloxy)-4-(4-((2-(3-(1,1-difluoroethyl)bicyclo[1.1.1]pentan-1-yl)-4,4-dimethylcyclohex-1-enyl)methyl)piperazin-1-yl)benzoate (Intermediate 33-6) was prepared following the procedure described in Step 6, Route A for Intermediate 28 using Intermediate 33-5 in place of Intermediate 28-5. LC/MS (ESI) m/z 647.3 [M+H]⁺.

Step 7: Intermediate 33 was prepared following the procedure described in Step 7 for Intermediate 32 using Intermediate 33-6 in place of Intermediate 32-6. LC/MS (ESI) m/z 591.3 [M+H]⁺.

### Intermediate 34

### (S)-4-(((1,4-dioxan-2-yl)methyl)amino)-3-nitrobenzenesulfonamide

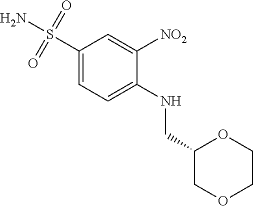 [SMILES: NS(=O)(=O)c1ccc(NC[C@H]2COCCO2)c([N+](=O)[O-])c1]

A solution of (S)-(1,4-dioxan-2-yl)methanamine hydrochloride (500 mg, 3.25 mmol) in THF (5 mL) was treated with 4-fluoro-3-nitrobenzenesulfonamide (501 mg, 2.20 mmol) and DIPEA (1.65 g, 13 mmol) and the mixture was heated to 45° C. After 16 h, the reaction was concentrated, triturated with MeOH, and filtered to provide Intermediate 34 (500 mg, 48%) as a yellow solid. LC/MS (ESI) m/z 318.4 [M+H]⁺.

### Intermediate 35

### (R)-4-((4-((2-((tert-butyldiphenylsilyl)oxy)ethyl)(methyl)amino)-1-(phenylthio)butan-2-yl)amino)-3-((trifluoromethyl) sulfonyl)benzenesulfonamide

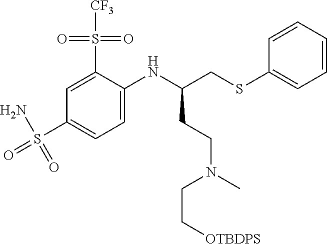

Intermediate 35 was prepared following a procedure described in WO2012/017251A1. LCMS (ESI) m/z 780.6 [M+H]⁺.

### Intermediate 36

### 4-(4-((2-(3-Chlorobicyclo[1.1.1]pentan-1-yl)-5,5-dimethylcyclohex-1-en-1-yl)methyl)piperazin-1-yl)benzoic acid

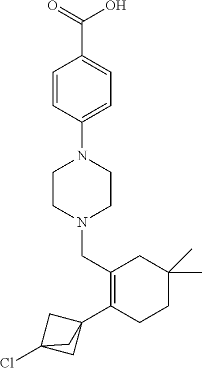 [SMILES: CC1(C)CCC(C23CC(Cl)(C2)C3)=C(CN2CCN(c3ccc(C(=O)O)cc3)CC2)C1]

Step 1: To a stirred solution of 3,3-dimethylpent-4-en-1-ol (18.5 g, 162.01 mmol) in DCM (100 mL), was added MsCl (13.54 mL, 175.0 mmol) followed by NEt₃ (33.87 mL, 243.0 mmol) at 0° C. and the reaction was warmed to rt. After 4 h, sat. aq. NaHCO₃ solution (100 mL) was added and the reaction was extracted with DCM (3×100 mL). The combined organic layers were dried over Na₂SO₄, filtered and concentrated to afford 3,3-dimethylpent-4-enyl methanesulfonate (Intermediate 36-1) (20.0 g, 64% yield) as a clear colorless oil. This was used in the next step without further purification. ¹H NMR (400 MHz, CDCl₃) δ 5.80-5.72 (m, 1H), 5.01-4.94 (m, 2H), 4.22-4.18 (m, 2H), 2.99 (s, 3H), 1.81-1.77 (m, 2H), 1.06 (s, 6H).

Step 2: To a pressure flask was added Intermediate 36-1 (20 g, 104.01 mmol) and NaI (46.77 g, 312.04 mmol) in acetone (100 mL). The flask was sealed and the reaction was stirred at 100° C. for 12 h. The reaction mixture was cooled to rt, diluted with water (250 mL) and extracted with Et₂O (3×200 mL). The combined organic layers were washed with sat. aq. Na₂S203, dried over Na₂SO₄, and evaporated to afford 5-iodo-3,3-dimethylpent-1-ene (Intermediate 36-2) (18 g, 77% yield) as a clear colorless oil. ¹H NMR (400 MHz, CDCl₃) δ 5.75-5.68 (m, 1H), 5.01-4.92 (m, 2H), 3.09-3.05 (m, 2H), 1.99-1.95 (m, 2H), 1.01 (s, 6H)

Step 3: 1-(3-Chlorobicyclo[1.1.1]pentan-1-yl)-4,4-dimethylhex-5-en-1-one (Intermediate 36-3) was prepared following the procedure described in Step 1 for Intermediate 26 by reacting 36-2 in place of 5-iodo-4,4-dimethylpent-1-ene. ¹H NMR (400 MHz, CDCl₃) δ 5.71-5.63 (m, 1H), 4.97-4.88 (m, 2H), 2.38 (s, 6H), 2.34-2.30 (m, 2H), 1.57-1.52 (m, 2H), 0.98 (s, 6H).

Step 4: Ozone gas was bubbled into a solution of Intermediate 36-3 (1.5 g, 6.63 mmol) in DCM (40 mL) at −78° C. until the solution turned a blue color (˜30 min). Then N2 gas was bubbled into the reaction mixture until it became colorless. PPh₃ (2.6 g, 9.94 mmol) was added in one portion and the reaction was warmed to rt. After 3 h, the reaction mixture was diluted with DCM (100 mL), washed with water (2×25 mL), and brine (50 mL). The organic layer was dried over Na₂SO₄, filtered and concentrated. The crude product was purified by column chromatography (SiO₂, EtOAc/pet. ether) to afford 5-(3-chlorobicyclo[1.1.1]pentan-1-yl)-2,2-dimethyl-5-oxopentanal (Intermediate 36-4) as a clear colorless oil (800 mg, 53% yield). ¹H NMR (400 MHz, CDCl₃) δ 9.41 (s, 1H), 2.39 (s, 6H), 2.38-2.33 (m, 2H), 1.77-1.72 (m, 2H), 1.05 (s, 6H).

Step 5: To a stirred solution of diethyl cyanomethylphosphonate (619 mg, 3.50 mmol) in toluene (10 mL) at 0° C. was added LiHMDS (1 M in toluene, 3.5 mL, 3.50 mmol). The reaction was then warmed to rt. After 30 min, the solution was added dropwise at −78° C. to a solution of Intermediate 36-4 (800 mg, 3.50 mmol) in toluene (10 mL). The reaction mixture was warmed to rt and stirred for 16 h at which point it was cooled to 0° C. and quenched with sat. aq. NH₄Cl (20 ml). The organic phase was separated and the aqueous phase was further extracted with DCM (3×50 mL). The combined organic layers were dried over Na₂SO₄, filtered and concentrated. The crude product was purified by column chromatography (SiO₂, EtOAc/pet. ether) to obtain (E)-7-(3-chlorobicyclo[1.1.1]pentan-1-yl)-4,4-dimethyl-7-oxohept-2-enenitrile (Intermediate 36-5) as a clear colorless oil (440 mg, 50% yield). LC/MS (ESI) m/z 252.4 [M+H]⁺.

Step 6: A solution of Intermediate 36-5 (440 mg, 1.75 mmol) in MeOH (10 mL) was treated with Pd/C (25 wt %, 110 mg) and stirred under an atmosphere of H₂ (1 atm) for 2 h. The reaction was then purged with N2, and filtered over Celite. The Celite plug was washed with MeOH (3×25 mL) and the combined organic layers were concentrated to provide 7-(3-chlorobicyclo[1.1.1]pentan-1-yl)-4,4-dimethyl-7-oxoheptanenitrile (Intermediate 36-6) as a clear colorless oil (360 mg, 81% yield). ¹H NMR (400 MHz, CDCl₃) 2.41 (s, 6H), δ 2.40-2.36 (m, 2H), 2.30-2.25 (m, 2H), 1.63-1.56 (m, 2H), 1.50-1.46 (m, 2H), 0.89 (s, 6H).

Step 7: 2-(3-Chlorobicyclo[1.1.1]pentan-1-yl)-5,5-dimethylcyclohex-1-ene-1-carbonitrile (Intermediate 36-7) was prepared following the procedure described in Step 4 for Intermediate 26 by reacting Intermediate 36-6 in place of Intermediate 26-3. LC/MS (ESI) m/z 236.4 [M+H]⁺.

Step 8: 5,5-Dimethyl-2-(3-methylbicyclo[1.1.1]pentan-1-yl)cyclohex-1-ene-1-carbaldehyde (Intermediate 36-8) was prepared following the procedure described in Step 5 for Intermediate 26 by reacting Intermediate 36-7 in place of Intermediate 26-4. ¹H NMR (400 MHz, CDCl₃) δ 10.17 (s, 1H), 2.46 (s, 6H), 2.44 (s, 2H), 2.03 (t, J=7.6 Hz, 2H), 1.42-1.37 (m, 2H), 0.86 (s, 6H).

Step 9: To a stirred solution of Intermediate 36-8 (85 mg, 0.361 mmol) in EtOH (3 mL) was added tert-Butyl 4-(piperazin-1-yl)benzoate (104 mg, 0.397 mmol) and AcOH (cat.). After 15 min, the reaction was cooled to 0° C., treated with NaCNBH₃ (33.6 mg, 0.535 mmol) and warmed to rt. After 16 h, the reaction was diluted with sat. aq. NaHCO₃ and extracted with DCM (3×15 mL). The combined organic layers were dried over Na₂SO₄, filtered and concentrated. The crude product was purified by column chromatography (SiO₂, EtOAc/pet. ether) to obtain tert-Butyl 4-(4-((2-(3-chlorobicyclo[1.1.1]pentan-1-yl)-5,5-dimethylcyclohex-1-en-1-yl)methyl)piperazin-1-yl)benzoate (Intermediate 36-9) as a white solid (80 mg, 50% yield). LC/MS (ESI) m/z 485.6 [M+H]⁺.

Step 10: To a stirred solution of Intermediate 36-9 (80 mg, 0.165 mmol) in DCM (3 mL) at 0° C. was added TFA (113 mg, 0.99 mmol). The reaction was warmed to rt and stirred for 3 h. The reaction was concentrated and then diluted with sat. aq. NaHCO₃ and extracted with DCM (3×10 mL). The combined organic layers were dried over Na₂SO₄, filtered and concentrated to obtain the Intermediate 36 as an off-white solid (60 mg, 85%). LC/MS (ESI) m/z 429.5 [M+H]⁺.

### Intermediate 37

### (R)-4-(4-(4-hydroxypiperidin-1-yl)-1-(phenylthio)butan-2-ylamino)-3-(trifluoromethylsulfonyl)benzenesulfonamide

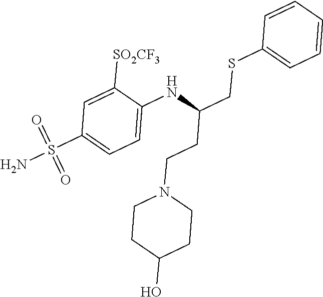

Step 1: To a stirred solution of (R)-3-((((9H-fluoren-9-yl)methoxy)carbonyl)amino)-4-(phenylthio)butanoic acid (6.8 g, 15.7 mmol) in DCM (70 mL) and DMF (10 mL) was added HATU (9.5 g, 25.12 mmol) followed by DIPEA (8.3 mL, 47.1 mmol) at 0° C. After 10 min, 4-hydroxypiperidine (2.4 g, 23.55 mmol) was added and temperature was raised to rt. After 16 h, the reaction was diluted with water and extracted with EtOAc. The combined organic layers were dried over Na₂SO₄, filtered, and concentrated. The crude product was purified by column chromatography (SiO₂ MeOH/DCM) to afford (R)-(9H-fluoren-9-yl)methyl-4-(4-hydroxypiperidin-1-yl)-4-oxo-1-(phenylthio)butan-2-ylcarbamate (Intermediate 37-1) (5.5 g, 68% yield) as a brown oil. LC/MS (ESI) m/z 517.6 [M+H]⁺.

Step 2: To a stirred solution of Intermediate 37-1 (2.75 g, 5.32 mmol) in CH₃CN (20 mL) at rt was added diethylamine (3.3 mL, 31.92 mmol) and stirred at rt. After 16 h, the reaction was concentrated and purified by column chromatography (neutral alumina, MeOH/DCM) to afford (R)-3-amino-1-(4-hydroxypiperidin-1-yl)-4-(phenylthio)butan-1-one (Intermediate 37-2) (900 mg, 57% yield) as a brown liquid. LC/MS (ESI) m/z 295.1 [M+H]⁺.

Step 3: To a stirred solution of Intermediate 37-2 (0.9 g, 3.06 mmol) in anhydrous THF (12 mL) at 0° C. was added BH₃ (1 M in THF, 9.18 mL, 9.18 mmol) and the temperature was raised to 45° C. After 16 h, the reaction was cooled to 0° C. and MeOH (30 ml) was added. After 1 hour, the reaction was concentrated and purified by column chromatography (C18, CH₃CN/Water) to afford (R)-1-(3-amino-4-(phenylthio)butyl)piperidin-4-ol (Intermediate 37-3) (305 mg, 36% yield) as an off-white semi solid. LC/MS (ESI) m/z 281.2 [M+H]⁺.

Step 4: To a stirred solution of Intermediate 37-3 (100 mg, 0.357 mmol) in DMF (1 mL) was added 4-fluoro-3-(trifluoromethylsulfonyl)benzenesulfonamide (99 mg, 0.32 mmol) followed by DIPEA (140 mg, 1.07 mmol) and the resulting reaction mixture was stirred at rt. After 16 h, the reaction was concentrated, diluted with water and extracted with 9:1 DCM:MeOH (2×10 mL). The combined organic layers were dried over Na₂SO₄, filtered and concentrated. The crude product was purified by trituration with EtOAc/Et₂O to afford Intermediate 37 (105 mg, 51% yield) as a white solid. LC/MS (ESI) m/z 568.1 [M+H]⁺.

### Intermediate 38

### 4-(4-((5,5-dimethyl-2-(3-methylbicyclo[1.1.1]pentan-1-yl)cyclohex-1-en-1-yl)methyl)piperazin-1-yl)benzoic acid

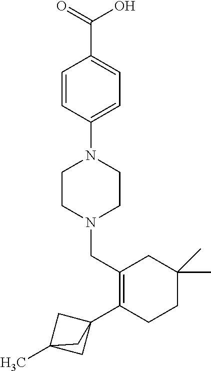

Step 1: 4,4-Dimethyl-1-(3-methylbicyclo[1.1.1]pentan-1-yl)hex-5-en-1-one (Intermediate 38-1) was prepared following the procedure described in Step 1 for Intermediate 26 using Intermediate 10 and Intermediate 36-2 in place of Intermediate 1 and 5-iodo-4,4-dimethylpent-1-ene. ¹H NMR (400 MHz, CDCl₃) δ 5.73-5.66 (m, 1H), 4.95-4.88 (m, 2H), 2.33-2.28 (m, 2H), 1.88 (s, 6H), 1.55-1.51 (m, 2H), 1.21 (s, 3H), 0.99 (s, 6H).

Step 2: 2,2-dimethyl-5-(3-methylbicyclo[1.1.1]pentan-1-yl)-5-oxopentanal (Intermediate 38-2) was prepared following the procedure described in Step 4 for Intermediate 36 using Intermediate 38-1 in place of Intermediate 36-3. 1H NMR (300 MHz, CDCl3) δ 9.41 (s, 1H), 2.36-2.30 (m, 2H), 1.88 (s, 6H), 1.79-1.71 (m, 2H), 1.18 (s, 3H), 1.05 (s, 6H).

Step 3: 4,4-dimethyl-7-(3-methylbicyclo[1.1.1]pentan-1-yl)-7-oxohept-2-enenitrile (Intermediate 38-3) was prepared following the procedure described in Step 5 for Intermediate 36 using Intermediate 38-2 in place of Intermediate 36-4. LC/MS (ESI) m/z 232.5 [M+H]⁺.

Step 4: 4,4-dimethyl-7-(3-methylbicyclo[1.1.1]pentan-1-yl)-7-oxoheptanenitrile (Intermediate 38-4) was prepared following the procedure described in Step 6 for Intermediate 36 using Intermediate 38-3 in place of Intermediate 36-5. ¹H NMR (400 MHz, CDCl₃) δ 2.38-2.33 (m, 2H), 2.29-2.25 (m, 2H), 1.90 (s, 6H), 1.62-1.58 (m, 2H), 1.48-1.44 (m, 2H), 1.19 (s, 3H), 0.90 (s, 6H).

Step 5: 5,5-dimethyl-2-(3-methylbicyclo[1.1.1]pentan-1-yl)cyclohex-1-ene-1-carbonitrile (Intermediate 38-5) was prepared following the procedure described in Step 4 for Intermediate 26 using Intermediate 38-4 in place of Intermediate 26-3. ¹H NMR (400 MHz, CDCl₃) δ 2.11-2.06 (m, 2H), 2.00-1.98 (m, 2H), 1.93 (s, 6H), 1.35 (t, J=6.4 Hz, 2H), 1.18 (s, 3H), 0.90 (s, 6H).

Step 6: 5,5-dimethyl-2-(3-methylbicyclo[1.1.1]pentan-1-yl)cyclohex-1-ene-1-carbaldehyde (Intermediate 38-6) was prepared following the procedure described in Step 5 for Intermediate 26 using Intermediate 38-5 in place of Intermediate 26-4. ¹H NMR (400 MHz, CDCl₃) δ 10.28 (s, 1H), 2.21-2.17 (m, 2H), 2.14 (br s, 2H), 2.00 (s, 6H), 1.35 (t, J=6.4 Hz, 2H), 1.20 (s, 3H), 0.88 (s, 6H).

Step 7: tert-Butyl 4-(4-((5,5-dimethyl-2-(3-methylbicyclo[1.1.1]pentan-1-yl)cyclohex-1-en-1-yl)methyl)piperazin-1-yl)benzoate (Intermediate 38-7) was prepared following the procedure described in Step 9 from Intermediate 36 using Intermediate 38-6 in place of Intermediate 36-8. LC/MS (ESI) m/z 465.6 [M+H]⁺.

Step 8: Intermediate 38 was prepared following the procedure described in Step 10 from Intermediate 36 by reacting Intermediate 38-7 in place of Intermediate 36-9. LC/MS (ESI) m/z 409.6 [M+H]⁺.

### Intermediate 39

### 4-(4-((4,4-Dimethyl-2-(3-methylbicyclo[1.1.1]pentan-1-yl)cyclohex-1-en-1-yl)methyl)piperazin-1-yl)benzoic acid

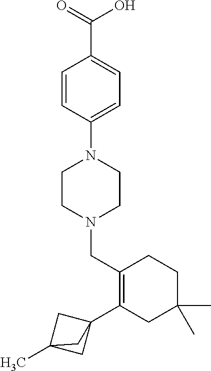

Step 1: To a stirred solution of methyl 4-(piperazin-1-yl)benzoate (1.68 g, 7.6 mmol) and Intermediate 22 (2.0 g, 9.15 mmol) in THF (20 mL) was added Na(OAc)₃BH (4.8 g, 22.8 mmol) at rt. After 16 h, the reaction was put in an ice batch and quenched with sat. aq. NaHCO₃ (25 mL). The reaction mixture was extracted with EtOAc (3×50 mL), dried over Na₂SO₄, filtered, and concentrated. The crude product was purified by column chromatography (SiO₂, EtOAc/pet. ether) to obtain methyl 4-(4-((4,4-dimethyl-2-(3-methylbicyclo[1.1.1]pentan-1-yl)cyclohex-1-en-1-yl)methyl)piperazin-1-yl)benzoate (Intermediate 39-1) as a white solid (1.5 g, 46% yield). LC/MS (ESI) m/z 423.2[M+H]⁺.

Step 2: Intermediate 39 was prepared following the procedure described in Step 5, Route B for Intermediate 28 using Intermediate 39-1 in place of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate. ¹H NMR (300 MHz, DMSO-d6) δ 12.25 (br s, 1H), 7.75 (d, J=9.0 Hz, 2H), 6.95 (d, J=9.0 Hz, 2H), 3.32-3.25 (m, 4H), 3.03 (s, 2H), 2.45-2.35 (m, 4H), 2.06-2.04 (m, 2H), 1.79 (s, 6H), 1.68 (s, 2H), 1.26 (t, J=6.3 Hz, 2H), 1.12 (s, 3H), 0.85 (s, 6H); LC/MS (ESI) m/z 409.5 [M+H]⁺.

### Intermediate 40

### 4-(4-((2-(3-ethylbicyclo[1.1.1]pentan-1-yl)-4,4-dimethylcyclohex-1-en-1-yl)methyl)piperazin-1-yl)benzoic acid

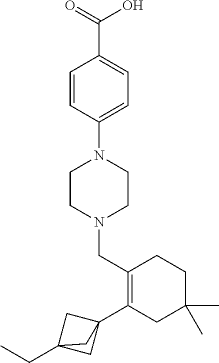 [SMILES: CCC12CC(C3=C(CN4CCN(c5ccc(C(=O)O)cc5)CC4)CCC(C)(C)C3)(C1)C2]

Step 1: Methyl 4-(4-((2-(3-ethylbicyclo[1.1.1]pentan-1-yl)-4,4-dimethylcyclohex-1-en-1-yl)methyl)piperazin-1-yl)benzoate (Intermediate 40-1) was prepared following the procedure described in Step 1 for Intermediate 39 using Intermediate 23 in place of Intermediate 22. LC/MS (ESI) m/z 437.3 [M+H]⁺.

Step 2: Intermediate 40 was prepared following the procedure described in Step 2 for Intermediate 39 using Intermediate 40-1 in place of Intermediate 39-1. LC/MS (ESI) m/z 423.3 [M+H]⁺.

### Intermediate 41

### 4-(4-((4,4-dimethyl-2-(3-(trifluoromethyl)bicyclo[1.1.1]pentan-1-yl)cyclohex-1-en-1-yl)methyl)piperazin-1-yl)benzoic acid

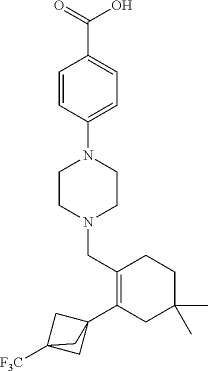 [SMILES: CC1(C)CCC(CN2CCN(c3ccc(C(=O)O)cc3)CC2)=C(C23CC(C(F)(F)F)(C2)C3)C1]

Step 1: To a stirred solution of Intermediate 25 (3.5 g, 12.85 mmol) in toluene was added titanium (IV) ethoxide (3.73 g, 16.36 mmol). After 30 min, a solution of methyl 4-(piperazin-1-yl) benzoate (2.35 g, 10.71 mmol) in toluene (20 mL) was added and the resulting reaction mixture was stirred at rt for 1 h. The reaction mixture was then cooled to 0° C., and Na(OAc)₃BH (6.9 g, 32.72 mmol) was added and the reaction was warmed to rt. After 16 h, the reaction was quenched with water (100 mL) at 0° C., and MTBE (200 mL) was added after 30 min. The reaction mixture was filtered over Celite and the collected solid was washed with DCM (2×100 mL). The combined organic layers were washed with sat. aq. NaHCO₃, brine, dried over Na₂SO₄, filtered and concentrated. The crude product was column chromatography (SiO₂, EtOAc/pet. ether) to afford methyl 4-(4-((4,4-dimethyl-2-(3-(trifluoromethyl)bicyclo[1.1.1]pentan-1-yl)cyclohex-1-en-1-yl)methyl)piperazin-1-yl)benzoate (Intermediate 41-1) (3.2 g, 63% yield) as a white solid. LC/MS (ESI) m/z 477.3 [M+H]⁺.

Step 2: Intermediate 41 was prepared following the procedure described in Step 2 for Intermediate 39 by reacting Intermediate 41-1 in place of Intermediate 39-1. LC/MS (ESI) m/z 463.2 [M+H]⁺.

### Intermediate 42

### 4-(4-((2-(3-(Difluoromethyl)bicyclo[1.1.1]pentan-1-yl)-4,4-dimethylcyclohex-1-en-1-yl)methyl)piperazin-1-yl)benzoic acid

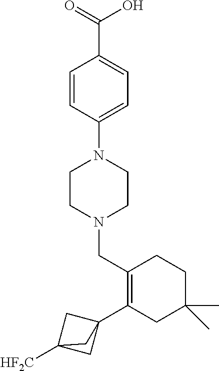

Step 1: Methyl 4-(4-((2-(3-(difluoromethyl)bicyclo[1.1.1]pentan-1-yl)-4,4-dimethylcyclohex-1-en-1-yl)methyl)piperazin-1-yl)benzoate (Intermediate 42-1) was prepared following the procedure described in Step 1 for Intermediate 39 using Intermediate 24 in place of Intermediate 22. ¹H NMR (400 MHz, DSMO-d₆) δ 7.77 (d, J=8.8 Hz, 2H), 6.97 (d, J=8.8 Hz, 2H), 6.01 (t, J=56.4 Hz, 1H), 3.77 (s, 3H), 3.35-3.20 (m, 4H), 3.00 (s, 2H), 2.42 (t, J=4.4 Hz, 4H), 2.10-2.01 (m, 2H), 1.90 (s, 6H), 1.71 (s, 2H), 1.27 (t, J=6.0 Hz, 2H), 0.86 (s, 6H); LC/MS (ESI) m/z 459.6 [M+H]⁺.

Step 2: Intermediate 42 was prepared following the procedure described in Step 2 for Intermediate 39 using Intermediate 42-1 in place of Intermediate 39-1. LC/MS (ESI) m/z 445.6 [M+H]⁺.

### Intermediate 43

### (R)-4-((4-morpholino-1-(phenylthio)butan-2-yl)amino)-3-nitrobenzenesulfonamide

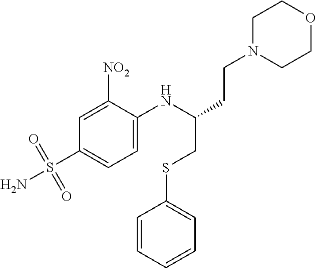 [SMILES: NS(=O)(=O)c1ccc(N[C@H](CCN2CCOCC2)CSc2ccccc2)c([N+](=O)[O-])c1]

To a solution of (R)-4-morpholino-1-(phenylthio)butan-2-amine dihydrochloride (900 mg, 2.6 mmol) in DMF (10 mL) was added 4-fluoro-3-nitrobenzenesulfonamide (56 mg, 2.53 mmol) followed by DIPEA (5.8 mL, 33.8 mmol) at rt. The reaction was then heated to 50° C. for 4 h. The reaction was cooled to rt, quenched with ice cold water (150 mL) and stirred at rt for 15 min. The mixture was then filtered and the collected solid was washed with n-pentane to afford Intermediate 43 (800 mg, 66%) as a yellow solid. LCMS (ESI) m/z 467.1 [M+H]⁺.

### Intermediate 44

### (R)-4-((4-(dimethylamino)-1-(phenylthio)butan-2-yl)amino)-3-((trifluoromethyl)sulfonyl)benzenesulfonamide

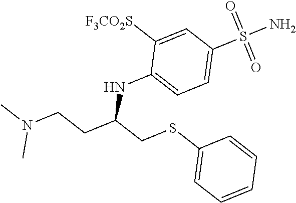

Intermediate 44 was prepared following a procedure described in WO200861208A2. LC/MS (ESI) m/z 512.2 [M+H]⁺.

### Intermediate 45

### (R)-4-((4-(4-(dimethylamino)piperidin-1-yl)-1-(phenylthio)butan-2-yl)amino)-3-((trifluoromethyl)sulfonyl)benzenesulfonamide

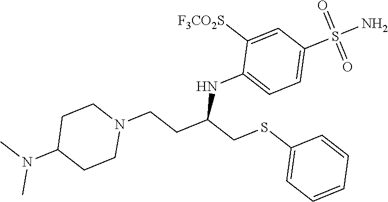

Step 1: To a stirred solution of N,N-dimethylpiperidin-4-amine (462.5 mg, 3.61 mmol), DMAP (367.80 mg, 3.01 mmol), and EDC.HCl (863.75 mg, 4.51 mmol) in DCM (20 mL) was added (R)-4-(phenylthio)-3-((4-sulfamoyl-2-((trifluoromethyl)sulfonyl)phenyl)amino)butanoic acid (prepared following a procedure described in WO2012017251A1) (1.5 g, 3.01 mmol) and Et₃N (0.84 mL, 6.02 mmol) at rt. After 15 min, the reaction was heated to 35° C. and stirred for 16 h. The reaction mixture was then cooled to rt, diluted with DCM (100 mL) and MeOH (10 mL) and washed with 10% CH₃CO₂H (aq.) (2×20 mL). The organic layer was then washed with 5% NaHCO₃(aq.) (20 mL) and 5% NaCl(aq.) (20 mL) and concentrated. The crude product was purified by column chromatography (C18, CH₃CN/H₂O) to provide (R)-4-((4-(4-(dimethylamino)piperidin-1-yl)-4-oxo-1-(phenylthio)butan-2-yl)amino)-3-((trifluoromethyl)sulfonyl)benzenesulfonamide (Intermediate 45-1) (686 mg, 37% yield) LC/MS (ESI) m/z 609.3[M+H]⁺.

Step 2: To a stirred solution of Intermediate 45-1 (800 mg, 1.31 mmol) in THF (15 mL) was added BH₃.THF (1M in THF, 6.57 mL, 6.57 mmol) at rt. The resulting reaction mixture was heated to 55° C. for 24 h in a sealed tube. The reaction was then cooled to rt, and treated with MeOH (8 mL) and conc. HCl (2 mL) and heated to 65° C. After 10 h. the reaction was concentrated, diluted with 2N NaOH solution and extracted with EtOAc. The combined organic layers were dried over Na₂SO₄, filtered and concentrated. The crude product was purified by column chromatography (C18, CH₃CN/H₂O) to afford Intermediate 45 (490 mg, 62% yield). LC/MS (ESI) m/z 595.3[M+H]⁺.

### Intermediate 46

### tert-butyl (R)-4-(4-(phenylthio)-3-((4-sulfamoyl-2-((trifluoromethyl)sulfonyl)phenyl)amino)butyl)piperazine-1-carboxylate

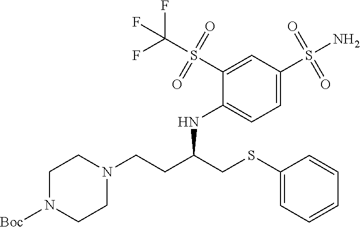 [SMILES: CC(C)(C)OC(=O)N1CCN(CC[C@H](CSc2ccccc2)Nc2ccc(S(N)(=O)=O)cc2S(=O)(=O)C(F)(F)F)CC1]

Step 1: (R)-tert-Butyl 4-(4-(phenylthio)-3-((4-sulfamoyl-2-((trifluoromethyl)sulfonyl)phenyl)-amino)butanoyl)piperazine-1-carboxylate (Intermediate 46-1) was prepared following the procedure described in Step 1 for Intermediate 45 using tert-butyl piperazine-1-carboxylate in place of N,N-dimethylpiperidin-4-amine. LC/MS (ESI) m/z 665.4 [M−H]⁻.

Step 2: Intermediate 46 was prepared following the procedure described in Step 2 for Intermediate 45 using Intermediate 46-1 in place of Intermediate 45-1. LC/MS (ESI) m/z 653.2 [M+H]⁺.

### Intermediate 47

### 7-(Diethoxymethyl)spiro[3.5]nonan-6-one

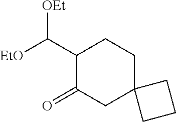 [SMILES: CCOC(OCC)C1CCC2(CCC2)CC1=O]

To a solution of triethyl orthoformate (7.28 ml, 43.79 mmol) in DCM (10 mL) at −30° C. was added BF₃.OEt₂ (6.75 ml, 54.72 mmol) dropwise over 20 min. The reaction mixture was warmed to 0° C. and stirred for 20 min. The reaction mixture was then cooled to −78° C. and spiro[3.5]nonan-6-one (3.0 g, 21.89 mmol) and N,N-diisopropylethylamine (11.4 ml, 35.7 mmol) were added and stirred for 90 min at the same temperature. The reaction was then carefully poured into a mixture of sat. aq. NaHCO₃ (20 mL) and DCM (30 mL). The resulting mixture was stirred for 15 min at rt and the organic layer was separated. The organic layer was washed with cold 1M H₂SO₄ (2×20 mL) and water. The organic layer was dried over Na₂SO₄, filtered and concentrated. The crude product was purified by column chromatography (SiO₂, Et₂O/pet. ether) to afford Intermediate 47 (3.00 g, 57% yield) as a colorless oil. 1H NMR (400 MHz, CDCl₃) δ 4.78 (d, J=6.4 Hz, 1H), 3.72-3.56 (m, 4H), 2.48-2.45 (m, 1H), 2.38 (d, J=1.2 Hz, 1H), 2.35 (d, J=0.8 Hz, 1H), 1.90-1.64 (m, 10H), 1.18 (t, J=6.8 Hz, 6H).

### Intermediate 48

### 7-(Diethoxymethyl)-6-(3-(difluoromethyl)bicyclo[1.1.1]pentan-1-yl)spiro[3.5]nonan-6-ol

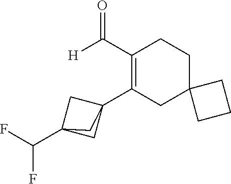 [SMILES: O=CC1=C(C23CC(C(F)F)(C2)C3)CC2(CCC2)CC1]

Step 1: To a stirred solution of Intermediate 24-2 (4.67 g, 19.15 mmol) in Et₂O (30 mL) under argon was added sec-BuLi (1.4 M in cyclohexane, 20.8 mL, 29.12 mmol) at −78° C. and the reaction was stirred for 10 minutes at the same temperature. The temperature was then warmed to 0° C. and stirred for 1 h. The reaction was cooled to −78° C. and a solution of Intermediate 47 (2 g, 8.32 mmol) in Et₂O (20 mL) was added dropwise for 5 minutes. The reaction was stirred at −78° C. for 1 h, and then warmed to 0° C. and stirred for 1 h. The reaction mixture was quenched with sat. aq. NH₄Cl solution (50 mL) at 0° C., and extracted with Et₂O (3×150 mL). The combined organic layers were dried over Na₂SO₄, filtered and concentrated to provide 7-(diethoxymethyl)-6-(3-(difluoromethyl)bicyclo[1.1.1]pentan-1-yl)spiro[3.5]nonan-6-ol (Intermediate 48-1) (1.5 g, crude) as a yellow oil. This was used in the next step without further purification.

Step 2: To a stirred solution of Intermediate 48-1 (1.5 g crude, 4.18 mmol) in 1,4-dioxane (30 mL) was added 2N HCl (aq.) (7 mL) and the resulting reaction mixture was stirred at 65-70° C. for 16 h. The reaction mixture was diluted with ice cold water (15 mL) and extracted with Et₂O (3×100 mL). The combined organic layers were dried over Na₂SO₄, filtered and concentrated. The product was purified by column chromatography (SiO₂, Et₂O/pet. ether) to afford Intermediate 48 (1 g, 45% yield over 2 steps) as a brown oil. ¹H NMR (400 MHz, CDCl₃) δ 10.21 (s, 1H), 5.74 (t, J=56.4 Hz, 1H), 2.22-2.19 (m, 2H), 2.18 (s, 6H), 1.93-1.86 (m, 4H), 1.83-1.65 (m, 4H), 1.63-1.56 (m, 2H).

### Intermediate 49

### 7-(diethoxymethyl)-6-(3-methylbicyclo[1.1.1]pentan-1-yl)spiro[3.5]nonan-6-ol

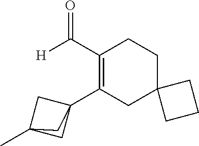 [SMILES: CC12CC(C3=C(C=O)CCC4(CCC4)C3)(C1)C2]

Step 1: 7-(diethoxymethyl)-6-(3-methylbicyclo[1.1.1]pentan-1-yl)spiro[3.5]nonan-6-ol (Intermediate 49-1) was prepared following the procedure described in Step 1 for Intermediate 48 using 1-iodo-3-methylbicyclo[1.1.1]pentane in place of Intermediate 24-2.

Step 2: Intermediate 49 was prepared following the procedure described in Step 2 for Intermediate 49 using Intermediate 49-1 in place of Intermediate 48-1. ¹H NMR (400 MHz, CDCl₃) δ 10.23 (s, 1H), 2.23-2.20 (m, 2H), 1.96 (s, 6H), 1.89-1.71 (m, 8H), 1.58-1.55 (m, 2H), 1.16 (s, 3H).

### Intermediate 50

### 2-((1H-pyrrolo[2,3-b]pyridin-5-yl)oxy)-4-(4-((6-(3-(difluoromethyl)bicyclo[1.1.1]pentan-1-yl)spiro[3.5]non-6-en-7-yl)methyl)piperazin-1-yl)benzoic acid

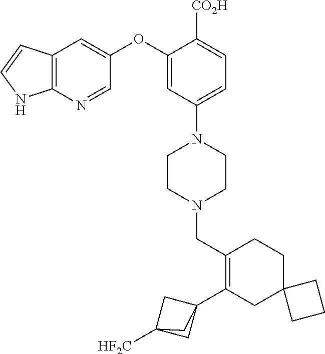

Step 1: Methyl 2-((1H-pyrrolo[2,3-b]pyridin-5-yl)oxy)-4-(4-((6-(3-(difluoromethyl)bicyclo[1.1.1]pentan-1-yl)spiro[3.5]non-6-en-7-yl)methyl)piperazin-1-yl)benzoate (Intermediate 50-1) was prepared following the procedure described in Step 1, Route C for Intermediate 28 using Intermediate 48 in place of Intermediate 22. LC/MS (ESI) m/z 603.5 [M+H]⁺.

Step 2: Intermediate 50 was prepared following the procedure described in Step 5, Route B for Intermediate 28 using Intermediate 50-1 in place of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate. LC/MS (ESI) m/z 589.3.

### Intermediate 51

### 2-((1H-pyrrolo[2,3-b]pyridin-5-yl)oxy)-4-(4-((6-(3-methylbicyclo[1.1.1]pentan-1-yl)spiro[3.5]non-6-en-7-yl)methyl)piperazin-1-yl)benzoic acid

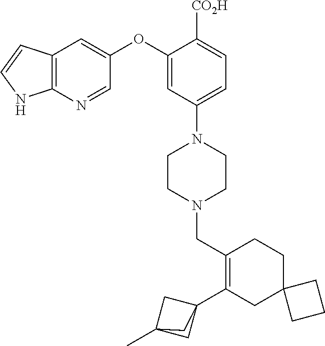 [SMILES: CC12CC(C3=C(CN4CCN(c5ccc(C(=O)O)c(Oc6cnc7[nH]ccc7c6)c5)CC4)CCC4(CCC4)C3)(C1)C2]

Step 1: Methyl 2-((1H-pyrrolo[2,3-b]pyridin-5-yl)oxy)-4-(4-((6-(3-methylbicyclo[1.1.1]pentan-1-yl)spiro[3.5]non-6-en-7-yl)methyl)piperazin-1-yl)benzoate: (Intermediate 51-1) was prepared following the procedure described in Step 1, Route C for Intermediate 28 using Intermediate 49 in place of Intermediate 22. LC/MS (ESI) m/z 567.3 [M+H]⁺.

Step 2: Intermediate 50 was prepared following the procedure described in Step 5, Route B for Intermediate 28 using Intermediate 51-1 in place of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate. LC/MS (ESI) m/z 553.3.

### Intermediate 52

### 4-(((4-fluorotetrahydro-2H-pyran-4-yl)methyl)amino)-3-nitrobenzenesulfonamide

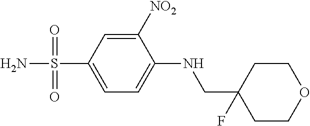 [SMILES: NS(=O)(=O)c1ccc(NCC2(F)CCOCC2)c([N+](=O)[O-])c1]

To a stirred solution of (4-fluorotetrahydro-2H-pyran-4-yl)methanamine (450 mg, 3.38 mmol) in THF (25 mL) was added 4-fluoro-3-nitrobenzenesulfonamide (669 mg, 3.04 mmol) followed by triethylamine (1.37 g, 13.52 mmol) at rt. After 16 h, the reaction was concentrated and triturated with EtOAc and Et₂O. The crude product was purified by column chromatography (C18, 0.1 M NH₄CO₃H(aq.):CH₃CN) to provide Intermediate 52 (220 mg, 21% yield) as a yellow solid. LC/MS (ESI) m/z 334.3[M+H]⁺.

### Intermediate 53

### 4-((4-fluorotetrahydro-2H-pyran-4-yl)methoxy)-3-nitrobenzenesulfonamide

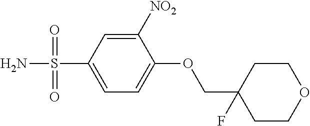 [SMILES: NS(=O)(=O)c1ccc(OCC2(F)CCOCC2)c([N+](=O)[O-])c1]

Intermediate 53 was prepared following the procedure described in Step 3 for Intermediate 7 by using (4-fluorotetrahydro-2H-pyran-4-yl)methanol in place of Intermediate 7-2. LC/MS (ESI) m/z 333.5 [M−H]⁻.

### Intermediate 54

### 4-((2-morpholinoethyl)amino)-3-nitrobenzenesulfonamide

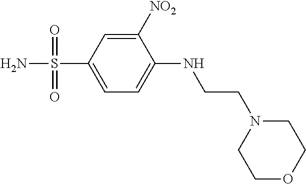 [SMILES: NS(=O)(=O)c1ccc(NCCN2CCOCC2)c([N+](=O)[O-])c1]

Intermediate 54 was prepared following a procedure described in a WO2010/065824. LC/MS (ESI) m/z 331.2 [M+H]⁺.

### Intermediate 55

### 3-nitro-4-((tetrahydro-2H-pyran-4-yl)methoxy)benzenesulfonamide

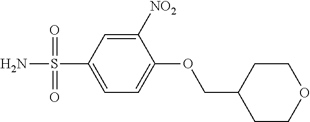 [SMILES: NS(=O)(=O)c1ccc(OCC2CCOCC2)c([N+](=O)[O-])c1]

Intermediate 55 was prepared following the procedure described in Step 3 for Intermediate 7 by using (tetrahydro-2H-pyran-4-yl)methanol in place of Intermediate 7-2. LC/MS (ESI) m/z 315.1 [M−H]⁻.

### Intermediate 56

### 4-(4-((2-(3-(difluoromethyl)bicyclo[11.1.1]pentan-1-yl)-5,5-dimethylcyclohex-1-en-1-yl)methyl)piperazin-1-yl)benzoic acid

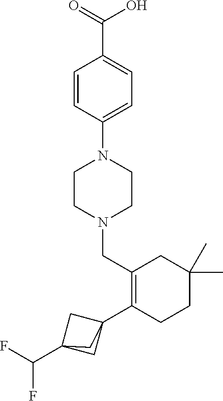 [SMILES: CC1(C)CCC(C23CC(C(F)F)(C2)C3)=C(CN2CCN(c3ccc(C(=O)O)cc3)CC2)C1]

Step 1: 2-(diethoxymethyl)-1-(3-(difluoromethyl)bicyclo[1.1.1]pentan-1-yl)-4,4-dimethylcyclohexanol (Intermediate 56-1) was prepared following the procedure described in Step 1, for Intermediate 25 using Intermediate 24-2 in place of i-iodo-3(trifluoromethyl)bicyclo[1.1.1]pentane and 2-(diethoxymethyl)-4,4-dimethylcyclohexanone in place of Intermediate 19. The crude product was used in the next step without purification.

Step 2: 2-(3-(difluoromethyl)bicyclo[1.1.1]pentan-1-yl)-5,5-dimethylcyclohex-1-enecarbaldehyde (Intermediate 56-2) was prepared following the procedure described in Step 2 for Intermediate 22, using Intermediate 56-1 in place of Intermediate 22-1. ¹H NMR (400 MHz, CDCl₃) δ 10.25 (br s, 1H), 5.74 (t, J=56.0 Hz, 1H), 2.23-2.21 (m, 2H), 2.20 (s, 6H), 2.03 (br s, 2H), 1.38 (t, J=6.4 Hz, 2H), 0.89 (s, 6H).

Step 3: To a stirred solution of methyl 4-(piperazin-1-yl)benzoate (389 mg, 1.77 mmol) in THF (10 mL) was added a solution of Intermediate 56-2 (450 mg, 1.77 mmol) in THF (5 mL) at rt. The reaction was stirred for 1 h, treated with Na(OAc)₃BH (1.12 g, 5.31 mmol) at 0° C., and then warmed to rt. After 16 h, MeOH (10 mL) was added and the reaction was stirred for 30 minutes. The reaction mixture was concentrated under reduced pressure, dissolved in DCM (20 mL) and washed with sat. aq. NaHCO₃ (3×10 mL). The organic layer was dried over Na₂SO₄, filtered, and concentrated. The crude product was purified by column chromatography (SiO₂, EtOAc/pet. ether) to afford methyl 4-(4-((2-(3-(difluoromethyl)bicyclo[1.1.1]pentan-1-yl)-5,5-dimethylcyclohex-1-en-1-yl)methyl)piperazin-1-yl)benzoate (Intermediate 56-3) (400 mg, 49% yield) as an off-white solid. LC/MS (ESI) m/z 459.2 [M+H]⁺.

Step 4: Intermediate 56 was prepared following the procedure described in Step 5, Route B for Intermediate 28 using Intermediate 56-3 in place of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate. LC/MS (ESI) m/z 445.4 [M+H]⁺.

### Intermediate 57

### (R)-4-((4-(3-hydroxyazetidin-1-yl)-1-(phenylthio)butan-2-yl)amino)-3-((trifluoromethyl)sulfonyl)benzenesulfonamide

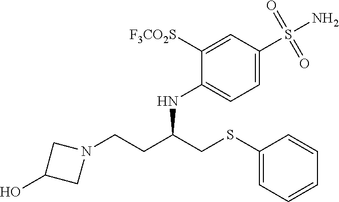

Step 1: To a solution of (R)-4-(phenylthio)-3-((4-sulfamoyl-2-((trifluoromethyl)sulfonyl)phenyl)amino)butanoic acid (1.5 g, 3.01 mmol) and 0-(Benzotriazol-1-yl)-N,N,N′,N′-tetramethyluronium tetrafluoroborate (TBTU) (1.09 g, 3.41 mmol) in DCM (3 mL) at 0° C. was added N-methylmorpholine (1.3 mL, 9.3 mmol) and DMF (1.5 mL). The reaction was warmed to rt and stirred for 0.5 h. The reaction mixture was then cooled to 0° C., and azetidin-3-ol (264 mg, 3.61 mmol) was added and the reaction was warmed to rt. After 16 h, the reaction was quenched with sat. aq. NaHCO₃ (50 mL) and extracted with EtOAc (3×100 mL). The combined organic layers were dried over Na₂SO₄, filtered and concentrated. The crude product was purified by column chromatography (SiO₂, MeOH/DCM) to afford (R)-4-((4-(3-hydroxyazetidin-1-yl)-4-oxo-1-(phenylthio)butan-2-yl)amino)-3-((trifluoro-methyl)sulfonyl)benzenesulfonamide (Intermediate 57-1) (1.00 g, 60% yield) as an off-white solid. LC/MS (ESI) m/z 554.1.

Step 2: To a stirred solution of Intermediate 57-1 (1.0 g, 1.80 mmol) in THF (20 mL) at 0° C. was added BH₃.THF (1 M in THF, 5.0 mL, 5 mmol) and the reaction was warmed to rt. After 1 h, the reaction mixture was heated to 55° C. and stirred for 16 h in a sealed tube. The reaction mixture was then cooled to 0° C., quenched with NH₃ (7.0 M in MeOH, 5 mL) at 0° C. and warmed to rt. After 16 h. the reaction was concentrated and purified by column chromatography (SiO₂, MeOH/DCM) to afford Intermediate 57 (500 mg, 51% yield) as an off-white solid. LC/MS (ESI) m/z 540.3 [M+H]⁺.

### Intermediate 58

### 4-(4-((6-(3-(difluoromethyl)bicyclo[1.1.1]pentan-1-yl)spiro[3.5]non-6-en-7-yl)methyl)piperazin-1-yl)benzoic acid

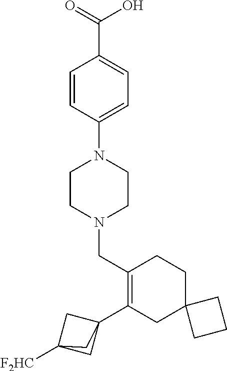 [SMILES: O=C(O)c1ccc(N2CCN(CC3=C(C45CC(C(F)F)(C4)C5)CC4(CCC4)CC3)CC2)cc1]

Step 1: Methyl 4-(4-((6-(3-(difluoromethyl)bicyclo[1.1.1]pentan-1-yl)spiro[3.5]non-6-en-7-yl)methyl)piperazin-1-yl)benzoate (Intermediate 58-1) was prepared following the procedure described in Step 3, for Intermediate 56 using Intermediate 48 in place of Intermediate 56-2. LC/MS (ESI) m/z 471.3 [M+H]⁺.

Step 2: Intermediate 58 was prepared following the procedure described in Step 5, Route B for Intermediate 28 using Intermediate 58-1 in place of methyl 2-((1H-pyrrolo[2,3-b]pyridin-5-yl)oxy)-4-(4-((4,4-dimethyl-2-(3-methylbicyclo[1.1.1]pentan-1-yl)cyclohex-1-en-1-yl)methyl)piperazin-1-yl)benzoate. LC/MS (ESI) m/z 457.5 [M+H]⁺.

### Intermediate 59

### (R)-4-((4-(4-(2-((tert-butyldiphenylsilyl)oxy)ethyl)piperazin-1-yl)-1-(phenylthio)butan-2-yl)amino)-3-((trifluoromethyl)sulfonyl)benzenesulfonamide

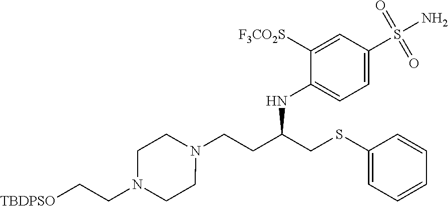

Step 1: (R)-4-((4-(4-(2-((tert-butyldiphenylsilyl)oxy)ethyl)piperazin-1-yl)-4-oxo-1-(phenylthio)butan-2-yl)amino)-3-((trifluoromethyl)sulfonyl)benzenesulfonamide (Intermediate 59-1) was prepared following the procedure described in Step 1 for Intermediate 45 using 1-(2-((tert-butyldiphenylsilyl)oxy)ethyl)piperazine in place of N,N-dimethylpiperidin-4-amine. LC/MS (ESI) m/z 849.3 [M+H]⁺.

Step 2: Intermediate 59 was prepared following the procedure described in Step 2, for Intermediate 57 using Intermediate 59-1 in place of Intermediate 57-1. LC/MS (ESI) m/z 835.0 [M+H]⁺.

### Intermediate 60

### (R)-4-((4-((2-((tert-butyldiphenylsilyl)oxy)ethyl)(ethyl)amino)-1-(phenylthio)butan-2-yl)amino)-3-((trifluoromethyl)sulfonyl)benzenesulfonamide

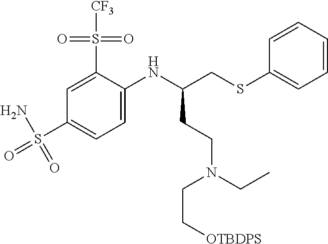

Step 1: 2-((tert-butyldiphenylsilyl)oxy)-N-ethylethanamine (Intermediate 60-1) was prepared following a procedure described in WO2012/017251A1. LC/MS (ESI) m/z 328.4 [M+H]⁺.

Step 2: To a stirred solution of (R)-4-(phenylthio)-3-((4-sulfamoyl-2-((trifluoromethyl)sulfonyl)phenyl)amino)butanoic acid (500 mg, 1.0 mmol) in CH₃CN (10 mL) at 0° C. was added Intermediate 60-1 (328 mg, 1.01 mmol) in CH₃CN (2 mL), followed by N-methyl imidazole (250 mg, 3.1 mmol) and N,N,N′,N′-tetramethylchloroformamidinium hexafluorophosphate (TCFH) (308 mg, 1.1 mmol). The reaction was warmed to rt and stirred for 16 h. The reaction was then diluted with water and extracted with EtOAc (3×100 mL). The combined organic layers were washed with sat. aq. NaHCO₃ (2×20 mL), water (2×10 mL) and then brine (2×20 mL). The organic layer was dried over Na₂SO₄, filtered and concentrated. The crude product was purified by column chromatography (SiO₂, EtOAc/pet. ether) to afford (R)-N-(2-((tert-butyldiphenylsilyl)oxy)ethyl)-N-ethyl-4-(phenylthio)-3-((4-sulfamoyl-2-((trifluoromethyl)sulfonyl)phenyl)amino)butanamide (Intermediate 60-2) (500 mg, 65% yield) as a yellow oil. LC/MS (ESI) m/z 808.4 [M+H]⁺.

Step 2: Intermediate 60 was prepared following the procedure described in Step 2, for Intermediate 57 using Intermediate 60-2 in place of Intermediate 57-1. LC/MS (ESI) m/z 794.8 [M+H]⁺.

### Intermediate 61

### 4-(((2R)-4-(3-Hydroxypyrrolidin-1-yl)-1-(phenylthio)butan-2-yl)amino)-3-((trifluoromethyl)sulfonyl)benzenesulfonamide

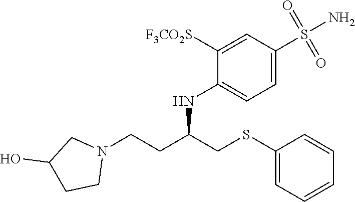

Step 1: 4-(((2R)-4-(3-Hydroxypyrrolidin-1-yl)-4-oxo-1-(phenylthio)butan-2-yl)amino)-3-((trifluoromethyl)sulfonyl)benzenesulfonamide (Intermediate 61-1) was prepared following the procedure described in Step 1, for Intermediate 45 using pyrrolidin-3-ol in place of N,N-dimethylpiperidin-4-amine. LC/MS (ESI) m/z 568.1 [M+H]⁺.

Step 2: Intermediate 61 was prepared following the procedure described in Step 2, for Intermediate 57 using Intermediate 61-1 in place of Intermediate 57-1. LC/MS (ESI) m/z 554.4 [M+H]⁺.

### Intermediate 62

### 2-((1H-pyrrolo[2,3-b]pyridin-5-yl)oxy)-4-(4-((2-(3-chlorobicyclo[1.1.1]pentan-1-yl)cyclohex-1-en-1-yl)methyl)piperazin-1-yl)benzoic acid

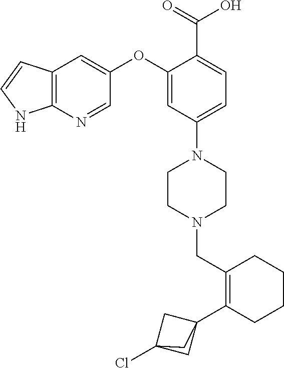 [SMILES: O=C(O)c1ccc(N2CCN(CC3=C(C45CC(Cl)(C4)C5)CCCC3)CC2)cc1Oc1cnc2[nH]ccc2c1]

Step 1: 1-(3-Chlorobicyclo[1.1.1]pentan-1-yl)hex-5-en-1-one (Intermediate 62-1) was prepared following the procedure described in Step 1 for Intermediate 26 using 5-bromopent-1-ene in place of 5-iodo-3,3-dimethylpent-1-ene. ¹H NMR (300 MHz, CDCl₃) δ 5.84-5.66 (m, 1H), 5.03-4.97 (m, 2H), 2.48 (s, 6H), 2.44 (t, J=7.2 Hz, 2H), 2.08-2.01 (m, 2H), 1.71-1.61 (m, 2H).

Step 2: E/Z-7-(3-chlorobicyclo[1.1.1]pentan-1-yl)-7-oxohept-2-enenitrile (Intermediate 62-2) was prepared following the procedure described in Step 2 for Intermediate 26 using Intermediate 62-1 in place of Intermediate 26-1. LC/MS (ESI) m/z 236.3 [M+H]⁺.

Step 3: 7-(3-Chlorobicyclo[1.1.1]pentan-1-yl)-7-oxoheptanenitrile (Intermediate 62-3) was prepared following the procedure described in Step 3 for Intermediate 26 using Intermediate 62-2 in place of Intermediate 26-2. ¹H NMR (400 MHz, CDCl₃) δ 2.47 (t, J=7.2 Hz, 2H), 2.40 (s, 6H), 2.35 (t, J=6.8 Hz, 2H), 1.70-1.62 (m, 2H), 1.61-1.55 (m, 2H), 1.48-1.41 (m, 2H).

Step 4: 2-(3-chlorobicyclo[1.1.1]pentan-1-yl)cyclohex-1-enecarbonitrile (Intermediate 62-4) was prepared following the procedure described in Step 4 for Intermediate 26 using Intermediate 62-3 in place of Intermediate 26-3. LC/MS (ESI) m/z 208.1 [M+H]⁺.

Step 5: 2-(3-chlorobicyclo[1.1.1]pentan-1-yl)cyclohex-1-enecarbaldehyde (Intermediate 62-5) was prepared following the procedure described in Step 5 for Intermediate 26 using Intermediate 62-4 in place of Intermediate 26-4. ¹H NMR (300 MHz, CDCl₃) δ 10.16 (s, 1H), 2.46 (s, 6H), 2.23-2.21 (m, 2H), 2.15-2.13 (m, 2H), 1.64-1.54 (m, 4H).

Step 6: tert-butyl 2-(1H-pyrrolo[2,3-b]pyridin-5-yloxy)-4-(4-((2-(3-chlorobicyclo[1.1.1]pentan-1-yl)cyclohex-1-enyl)methyl)piperazin-1-yl)benzoate (Intermediate 62-6) was prepared following the procedure described in Step 6, Route A for Intermediate 28 using Intermediate 62-5 in place of Intermediate 22. LC/MS (ESI) m/z 589.3 [M+H]⁺.

Step 7: Intermediate 62 was prepared following the procedure described in Step 7, for Intermediate 32, using Intermediate 62-6 in place of Intermediate 32-6. LC/MS (ESI) m/z 533.3 [M+H]⁺.

### Intermediate 63

### (R)-4-((4-(4-methylpiperazin-1-yl)-1-(phenylthio)butan-2-yl)amino)-3-((trifluoromethyl)sulfonyl)benzenesulfonamide

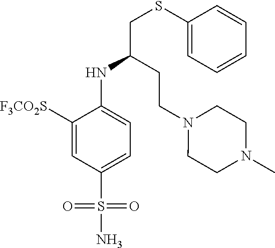

To a stirred solution of (R)-4-((4-oxo-1-(phenylthio)butan-2-yl)amino)-3-((trifluoromethyl)sulfonyl)benzenesulfonamide (prepared following a procedure described in WO2012017251A1) (250 mg, 0.518 mmol) in THF (10 mL) was added N-methyl piperizine (51 mg, 0.518 mmol) at rt. After 1 h, the reaction was cooled to 0° C., and Na(OAc)₃BH (329 mg, 1.55 mmol) was added, and the reaction was warmed to rt and stirred for 16 h. The reaction mixture was quenched with sat. aq. NaHCO₃ and extracted with EtOAc (3×30 mL). The combined organic layers were dried over Na₂SO₄, filtered and concentrated. The crude product was purified by column chromatography (SiO₂, MeOH/DCM) to afford Intermediate 63 (200 mg, 68% yield) as pale yellow oil. LC/MS (ESI) m/z 567.4 [M+H]⁺.

### Intermediate 64

### (R)-4-((4-(4-methoxypiperidin-1-yl)-1-(phenylthio)butan-2-yl)amino)-3-((trifluoromethyl)sulfonyl)benzenesulfonamide

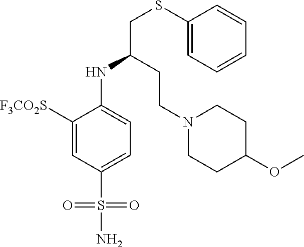

Intermediate 64 was prepared following the procedure described for Intermediate 63 using 4-methoxypiperidine in place of N-methyl piperizine. LC/MS (ESI) m/z 582.1 [M+H]⁺.

### Intermediate 65

### ((R)-4-((1-(phenylthio)-4-(pyrrolidin-1-yl)butan-2-yl)amino)-3-((trifluoromethyl)sulfonyl)benzenesulfonamide

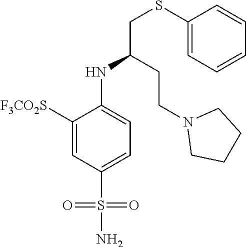

Intermediate 65 was prepared following the procedure described for Intermediate 63 using pyrrolidine in place of N-methyl piperizine. ¹H NMR (400 MHz, DMSO-d₆) δ 7.98 (d, J=2.0 Hz, 1H), 7.84 (dd, J=9.6, 2.0 Hz, 1H), 7.37-7.28 (m, 6H), 7.23-7.19 (m, 1H), 7.12 (d, J=8.4 Hz, 1H), 7.03 (d, J=9.6 Hz, 1H), 4.11-4.07 (m, 1H), 3.39-3.24 (m, 2H), 2.56-2.31 (m, 6H), 1.93-1.90 (m, 1H), 1.81-1.78 (m, 1H), 1.68-1.65 (m, 4H); LC/MS (ESI) m/z 538.4 [M+H]⁺.

### Intermediate 66

### (R)-4-((4-(4-Methoxy-4-methylpiperidin-1-yl)-1-(phenylthio)butan-2-yl)amino)-3-((trifluoromethyl)sulfonyl)benzenesulfonamide

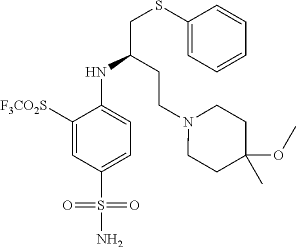

Intermediate 66 was prepared following the procedure described for Intermediate 63 using 4-methoxy-4-methylpiperidine in place of N-methyl piperizine. LC/MS (ESI) m/z 596.3 [M+H]⁺.

### Intermediate 67

### 4-(((R)-4-((S)-2-(((tert-butyldiphenylsilyl)oxy)methyl)morpholino)-1-(phenylthio)butan-2-yl)amino)-3-((trifluoromethyl)sulfonyl)benzenesulfonamide

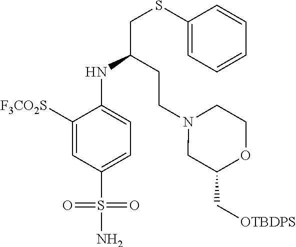

Step 1: (4-(((R)-4-((S)-2-(hydroxymethyl)morpholino)-1-(phenylthio)butan-2-yl)amino)-3-((trifluoromethyl)sulfonyl)benzenesulfonamide (Intermediate 67-1) was prepared following the procedure described for Intermediate 63 using (S)-morpholin-2-ylmethanol in place of N-methyl piperizine. LC/MS (ESI) m/z 584.2 [M+H]⁺.

Step 2: To a solution of Intermediate 67-1 (200 mg, 0.34 mmol) in DCM (10 mL) was added imidazole (70 mg, 1.02 mmol) and TBDPSCl (0.17 mL, 0.68 mmol) at 0° C. The reaction was warmed to rt and stirred for 16 h. The reaction mixture was then diluted with DCM (50 mL), washed with sat. aq. NaHCO₃ (50 mL), 5% NaCl(aq.) solution (100 mL), dried over Na₂SO₄ and concentrated. The crude product was purified by column chromatography (SiO₂, MeOH/DCM) to afford Intermediate 67 (170 mg, 60% yield) as an off white solid. LC/MS (ESI) m/z 822.2 [M+H]⁺.

### Intermediate 68

### 4-(((R)-4-((R)-2-(Hydroxymethyl)morpholino)-1-(phenylthio)butan-2-yl)amino)-3-((trifluoromethyl)sulfonyl)benzenesulfonamide

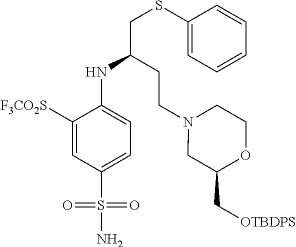

Step 1: (4-(((R)-4-((R)-2-(hydroxymethyl)morpholino)-1-(phenylthio)butan-2-yl)amino)-3-((trifluoromethyl)sulfonyl)benzenesulfonamide (Intermediate 68-1) was prepared following the procedure described for Intermediate 63 using (R)-morpholin-2-ylmethanol in place of N-methyl piperizine. LC/MS (ESI) m/z 584.1 [M+H]⁺.

Step 2: Intermediate 68 was prepared following the procedure described in Step 2 for Intermediate 67 using Intermediate 68-1 in place of Intermediate 67-1. LC/MS (ESI) m/z 822.3 [M+H]⁺.

### Intermediate 69

### (R)-methyl 4-methyl-1-(4-(phenylthio)-3-((4-sulfamoyl-2-((trifluoromethyl)sulfonyl)phenyl)amino)butyl)piperidine-4-carboxylate

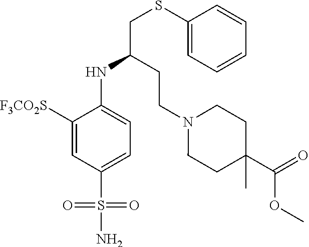

Intermediate 69 was prepared following the procedure described for Intermediate 63 using 4-methylpiperidine-4-carboxylate in place of N-methyl piperizine. LC/MS (ESI) m/z 624.1 [M+H]⁺.

### Intermediate 70

### (R)-4-((4-(4-ethoxypiperidin-1-yl)-1-(phenylthio)butan-2-yl)amino)-3-((trifluoromethyl)sulfonyl)benzenesulfonamide

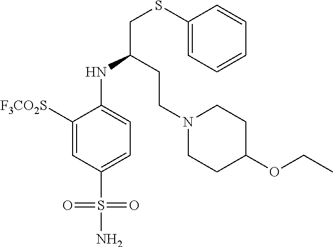

Intermediate 70 was prepared following the procedure described for Intermediate 63 using 4-ethoxypiperidine in place of N-methyl piperizine. LC/MS (ESI) m/z 596.3 [M+H]⁺.

### Intermediate 71

### (R)-4-((4-(4-isopropoxypiperidin-1-yl)-1-(phenylthio)butan-2-yl)amino)-3-((trifluoromethyl)sulfonyl)benzenesulfonamide

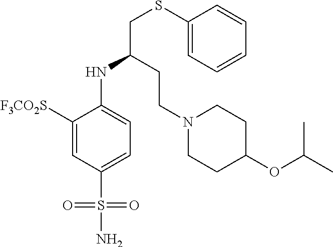

Intermediate 71 was prepared following the procedure described for Intermediate 63 using 4-isopropoxypiperidine in place of N-methyl piperizine. LC/MS (ESI) m/z 610.2 [M+H]⁺.

### Intermediate 72

### (R)-4-((4-(4-isopropylpiperazin-1-yl)-1-(phenylthio)butan-2-yl)amino)-3-((trifluoromethyl)sulfonyl)benzenesulfonamide

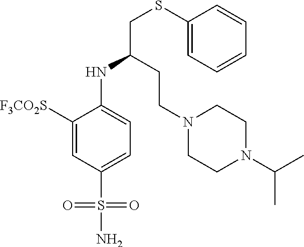

Intermediate 72 was prepared following the procedure described for Intermediate 63 using 1-isopropylpiperazine in place of N-methyl piperizine. LC/MS (ESI) m/z 595.1 [M+H]⁺.

### General Procedure A: Acyl Sulfonamide Formation

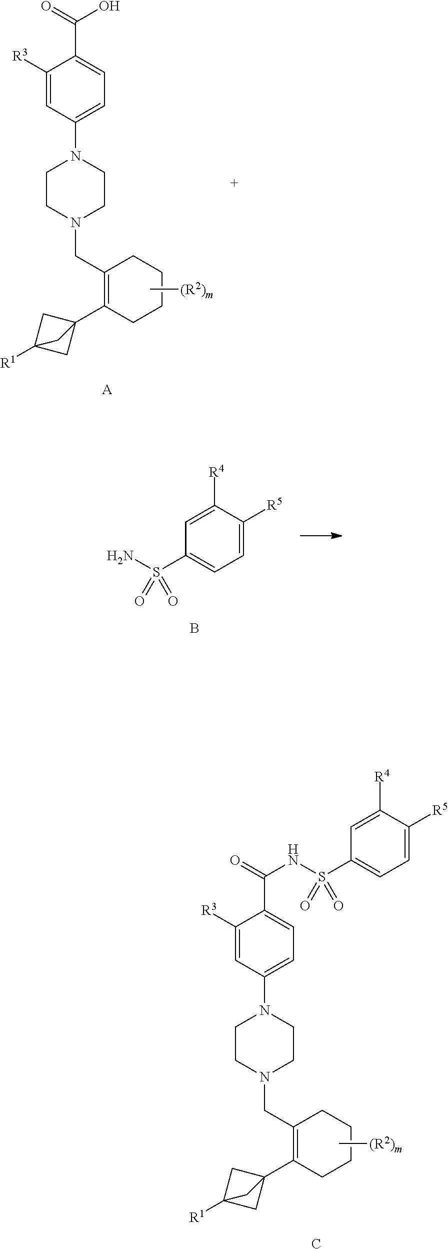

To a solution of corresponding sulfonamide B or acid A (1.0-1.2 equiv. Note #1) in DCM (0.01-0.1 M) at 0° C. was added EDC.HCl (1-2.5 equiv.) followed by DMAP (1-2 equiv.). After 10 min, the appropriate acid A or sulfonamide B (1-1.5 equiv. Note #1) and N-methylmorpholine (2-4 equiv. Note #2) were added at 0° C. and the reaction was warmed to rt or to 35° C. Upon completion as determined by LCMS (or TLC), water was added and the reaction was extracted with DCM. The combined organic layers were dried over Na₂SO₄ and concentrated. The crude product C was either purified by 1) column chromatography (SiO₂), 2) HPLC (10 mM NH₄CO₃H(aq): CH₃CN or MeOH) or 3) trituration with an organic solvent.

Note #1: In some instances, the TFA salt of acid A was used.

Note #2: In some instances, N-methylmorpholine was not added.

### General Procedure B: Acyl Sulfonamide Formation

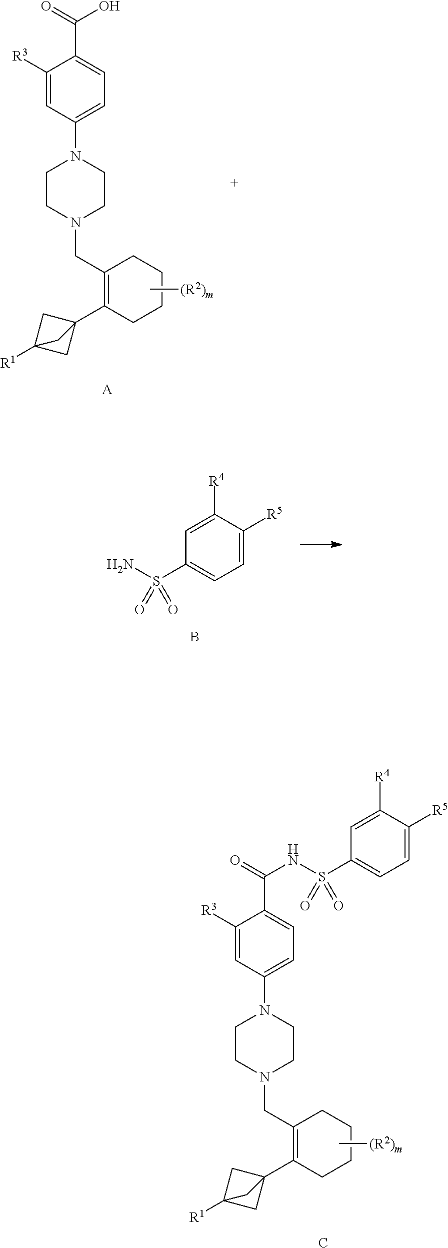

To a solution of corresponding sulfonamide B (1.0 equiv) in DCM (0.01-0.1 M) at rt was added EDC.HCl (1.5-1.75 equiv.) and DMAP (1-2.5 equiv.). In a separate flask, the appropriate acid A (1-1.1 equiv.) was dissolved in DCM (0.02-0.1M) was treated with Et₃N (2-2.5 equiv). (Notes #1 and 2). The acid solution was added to the sulfonamide suspension and stirred at rt and/or heated to 35° C. Upon completion as determined by LCMS, N,N-dimethylethylenediamine (2-2.5 equiv., Note #3) was added to the reaction mixture and the reaction was stirred for 90 min. The reaction mixture was then washed with 10% aq. AcOH (Note #4), 5% NaHCO₃(aq.) and then with 5% NaCl (aq.). The organic layer was dried, filtered and concentrated. The crude product C was either purified by 1) column chromatography (SiO₂), 2) HPLC (10 mM NH₄CO₃H(aq): CH₃CN or MeOH), or 3) trituration with an organic solvent.

Note #1: In some instances, DCM was not added.

Note #2: In some instances, Et₃N was added to the flask containing sulfonamide B.

Note #3: In some instances, N,N-dimethylethylenediamine was not added during the workup.

Note #4: In some instances, the organic layer was diluted with DCM and MeOH to solubilize the crude product.

The compounds of Examples 1-97 were synthesized using the intermediates described above and as described in PCT Publication Nos. WO 2019/139899, WO 2019/139900, WO 2019/139902, and WO 2019/139907.

### Example 98

### (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((4-(4-methylpiperazin-1-yl)-1-(phenylthio)butan-2-yl)amino)-3-((trifluoromethyl)sulfonyl)phenyl)sulfonyl)benzamide

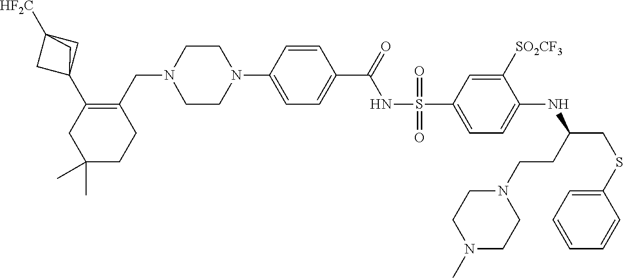

Representative example of General Procedure B: To a stirred solution of Intermediate 63 (127 mg, 0.22 mmol), DMAP (27 mg, 0.22 mmol), and EDC.HCl (64 mg, 0.33 mmol) in DCM (5 mL), was added a mixture of Intermediate 42 (100 mg, 0.22 mmol) and Et₃N (63 μL, 0.45 mmol) at rt. The resulting reaction mixture was heated to 35° C. and stirred for 16 h. The reaction mixture was cooled to rt, diluted with DCM (50 mL) and MeOH (5 mL), washed with 10% AcOH(aq.) (2×20 mL), 5% NaHCO₃(aq.) (2×10 mL), and 5% NaCl(aq.) (2×10 mL). The organic layer was dried over anhydrous Na₂SO₄, filtered and concentrated. The crude product was purified by column chromatography (SiO₂, MeOH/DCM) followed by trituration with Et₂O and pentane to afford Example 98 (24 mg, 11% yield) as an off white solid. LC/MS (ESI) m/z 993.5 [M+H]⁺.

### Example 99

### (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((4-(4-methoxypiperidin-1-yl)-1-(phenylthio)butan-2-yl)amino)-3-((trifluoromethyl)sulfonyl)phenyl)sulfonyl)benzamide

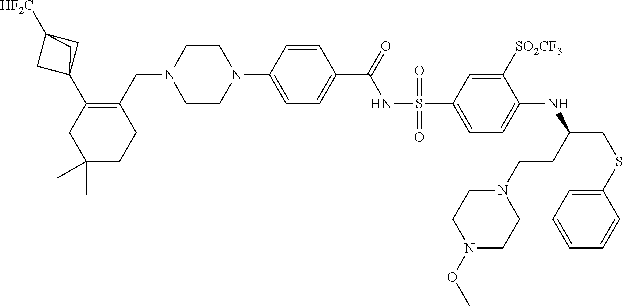

Example 99 was prepared following General Procedure B using Intermediate 42 and Intermediate 64. LC/MS (ESI) m/z 1008.4[M+H]⁺.

### Example 100

### (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((1-(phenylthio)-4-(pyrrolidin-1-yl)butan-2-yl)amino)-3-((trifluoromethyl)sulfonyl)phenyl)sulfonyl)benzamide

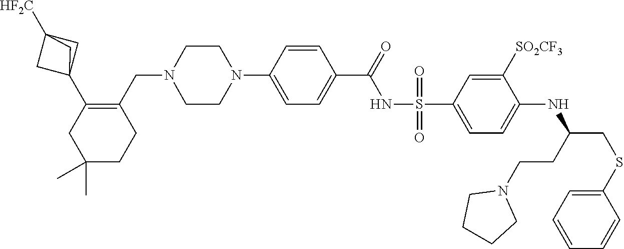

Example 100 was prepared following General Procedure B using Intermediate 42 and Intermediate 65. ¹H NMR (300 MHz, DMSO-d₆) δ 9.70 (br s, 1H), 8.08 (s, 1H), 7.97 (d, J=8.4 Hz, 1H), 7.72 (d, J=8.1 Hz, 2H), 7.36-7.20 (m, 5H), 6.92-6.68 (m, 4H), 6.01 (t, J=56.7 Hz, 1H), 4.02 (br s, 1H), 3.28-2.85 (m, 14H), 2.43 (br s, 4H), 2.06-1.85 (m, 14H), 1.70 (s, 2H), 1.28-1.24 (m, 2H), 0.86 (s, 6H); LC/MS (ESI) m/z 964.5 [M+H]⁺.

### Example 101

### (R)-4-(4-((2-(3-(Difluoromethyl)bicyclo[1.1.1]pentan-1-yl)-4,4-dimethylcyclohex-1-en-1-yl)methyl)piperazin-1-yl)-N-((4-((4-(4-methoxy-4-methylpiperidin-1-yl)-1-(phenylthio)butan-2-yl)amino)-3-((trifluoromethyl)sulfonyl)phenyl)sulfonyl benzamide

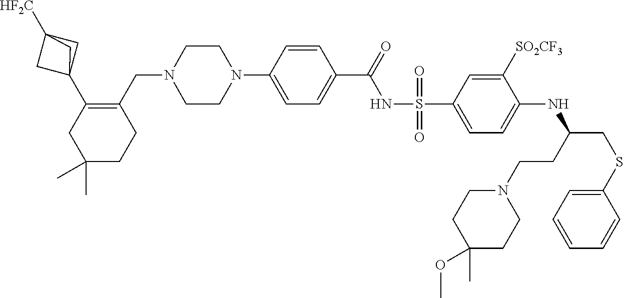

Example 101 was prepared following General Procedure B using Intermediate 42 and Intermediate 66. LC/MS (ESI) m/z 1022.3 [M+H]⁺.

### Example 102

### (N-((4-(((R)-4-((S)-2-(((tert-butyldiphenylsilyl)oxy)methyl)morpholino)-1-(phenylthio)butan-2-yl)amino)-3-((trifluoromethyl)sulfonyl)phenyl)sulfonyl)-4-(4-((2-(3-(difluoromethyl)bicyclo [1.1.1]pentan-1-yl)-4,4-dimethylcyclohex-1-en-1-yl)methyl)piperazin-1-yl)benzamide

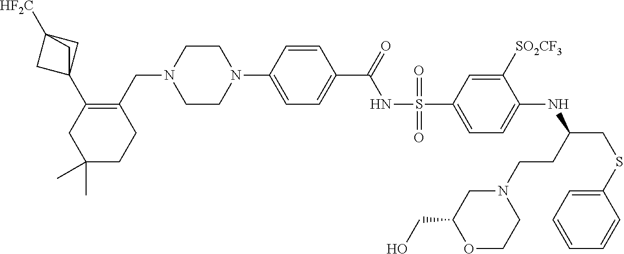

Step 1: (N-((4-(((R)-4-((S)-2-(((tert-butyldiphenylsilyl)oxy)methyl)morpholino)-1-(phenylthio)butan-2-yl)amino)-3-((trifluoromethyl)sulfonyl)phenyl)sulfonyl)-4-(4-((2-(3-(difluoromethyl)bicyclo[1.1.1]pentan-1-yl)-4,4-dimethylcyclohex-1-en-1-yl)methyl)piperazin-1-yl)benzamide (Example 102-1) was prepared following General Procedure B using Intermediate 42 and Intermediate 67. LC/MS (ESI) m/z 624.7 [M+2H]²⁺.

Step 2: To a stirred solution of Example 102-1 (50 mg, 0.04 mmol) in 1,4-dioxane (3 mL) and H₂O (0.5 mL) at 0° C., was added HCl (4M in 1,4-dioxane, 0.5 mL). The reaction was warmed to rt and stirred for 16 h. The reaction was quenched with sat. aq. NaHCO₃ (15 mL) and extracted with EtOAc (2×50 mL). The combined organic layers were dried over anhydrous Na₂SO₄, filtered and concentrated. The crude product was purified by HPLC (40:60 to 0:100 10 mM NH₄CO₃H(aq.)/CH₃CN) to provide Example 102 (5 mg, 12% yield) as an off-white solid. LC/MS (ESI) m/z 1010.4 [M+H]⁺.

### Example 103

### N-((4-(((R)-4-((R)-2-(((tert-Butyldiphenylsilyl)oxy)methyl)morpholino)-1-(phenylthio)butan-2-yl)amino)-3-((trifluoromethyl)sulfonyl)phenyl)sulfonyl)-4-(4-((2-(3-(difluoromethyl)bicyclo[1.1.1]pentan-1-yl)-4,4-dimethylcyclohex-1-en-1-1 methyl)piperazin-1-yl)benzamide

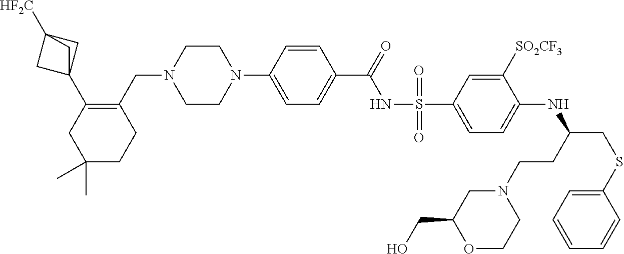

Step 1: (N-((4-(((R)-4-((R)-2-(((tert-butyldiphenylsilyl)oxy)methyl)morpholino)-1-(phenylthio)butan-2-yl)amino)-3-((trifluoromethyl)sulfonyl)phenyl)sulfonyl)-4-(4-((2-(3-(difluoromethyl)bicyclo[1.1.1]pentan-1-yl)-4,4-dimethylcyclohex-1-en-1-yl)methyl)piperazin-1-yl)benzamide (Example 103-1) was prepared following General Procedure B using Intermediate 42 and Intermediate 68. LC/MS (ESI) m/z 1248.4 [M+H]⁺.

Step 2: Example 103 was prepared following the procedure described in Step 2 for Example 102 using Example 103-1 in place of Example 102-1. LC/MS (ESI) m/z 1010.4 [M+H]⁺.

### Example 104

### (R)-Methyl 1-(3-((4-(N-(4-(4-((2-(3-(difluoromethyl)bicyclo[1.1.1]pentan-1-yl)-4,4-dimethylcyclohex-1-en-1-yl)methyl)piperazin-1-yl)benzoyl)sulfamoyl)-2-((trifluoromethyl)sulfonyl)phenyl)amino)-4-(phenylthio)butyl)-4-methylpiperidine-4-carboxylate

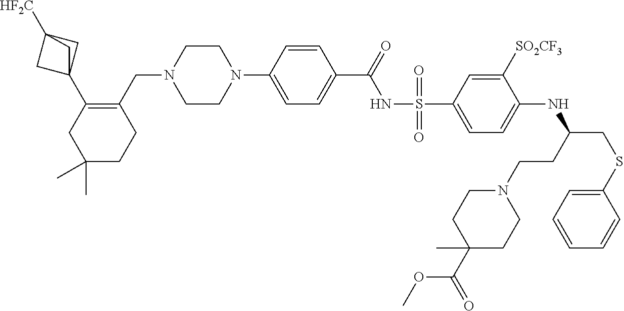

Example 104 was prepared following General Procedure B using Intermediate 42 and Intermediate 69. LC/MS (ESI) m/z 1050.4 [M+H]⁺.

### Example 105

### (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((4-(4-ethoxypiperidin-1-yl)-1-(phenylthio)butan-2-yl)amino)-((trifluoromethyl)sulfonyl)phenyl)sulfonyl)benzamide

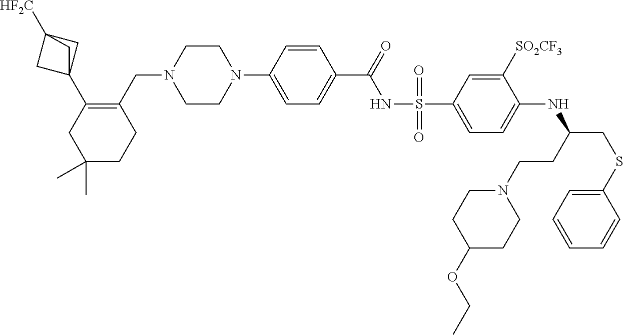

Example 105 was prepared following General Procedure B using Intermediate 42 and Intermediate 70. LC/MS (ESI) m/z 1022.3 [M+H]⁺.

### Example 106

### ((R)-4-(4-((2-(3-(difluoromethyl)bicyclo[1.1.1]pentan-1-yl)-4,4-dimethylcyclohex-1-en-1-yl)methyl)piperazin-1-yl)-N-((4-((4-(4-isopropoxypiperidin-1-yl)-1-(phenylthio)butan-2-yl)amino)-3-((trifluoromethyl)sulfonyl)phenyl)sulfonyl)benzamide

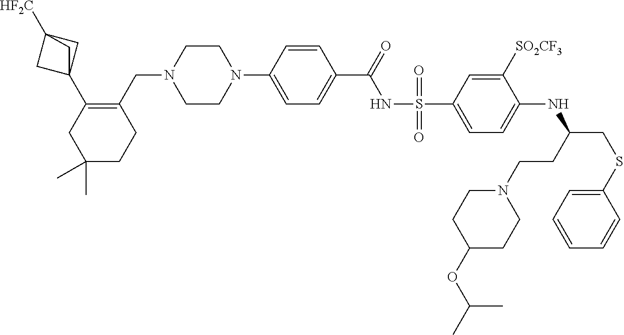

Example 106 was prepared following General Procedure B using Intermediate 42 and Intermediate 71. LC/MS (ESI) m/z 1036.6 [M+H]⁺.

### Example 107

### (R)-4-(4-((2-(3-(difluoromethyl)bicyclo[1.1.1]pentan-1-yl)-4,4-dimethylcyclohex-1-en-1-yl)methyl)piperazin-1-yl)-N-((4-((4-(4-isopropylpiperazin-1-yl)-1-(phenylthio)butan-2-yl)amino)-3-((trifluoromethyl)sulfonyl)phenyl)sulfonyl)benzamide

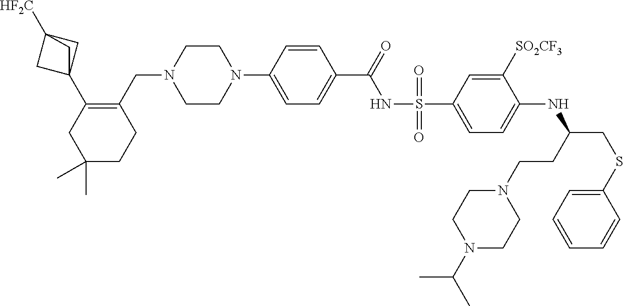

Example 107 was prepared following General Procedure B using Intermediate 42 and Intermediate 72. LC/MS (ESI) m/z 1021.6 [M+H]⁺.

### Example 108

### (R)-4-(4-((2-(3-ethylbicyclo[1.1.1]pentan-1-yl)-4,4-dimethylcyclohex-1-en-1-yl)methyl)piperazin-1-yl)-N-((4-((4-(4-methoxypiperidin-1-yl)-1-(phenylthio)butan-2-yl)amino)-3-((triluoromethyl)sulfonyl)phenyl)sulfonyl)benzamide

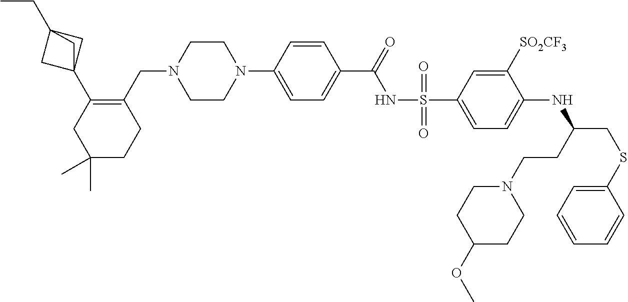

Example 108 was prepare following General Procedure B using Intermediate 40 and Intermediate 64. LC/MS (ESI) m/z 986.6 [M+H]⁺.

### Example 109

Preparation of a nanoparticle pharmaceutical composition comprising albumin and Example 61: 100 μL of a human albumin solution (100 mg/mL in H₂O) was added to a 1.5 mL microcentrifuge tube and diluted with 500 μL of water and 100 μL of 10 mmol NaHCO₃(aq.). After vortexing for 5 seconds, 100 μL of a 30 mg/mL DMSO solution of Example 61 was added quickly to the albumin solution and immediately vortexed for 10-15 seconds. The crude nanoparticle solution was then purified by size exclusion chromatography (GE Health Sciences™ PD-10) and the particle size (Analysis by Number, Malvern Nano ZS) was determined to be 67 nm after 2 hours following purification. The purified nanoparticle solution was diluted with a 20 mM solution of sodium N-acetyl-DL-trytophanate and sodium caprylate and lyophilized. The particle size of the lyophilized material after being reconstituted in water was 153 nm.

### Example 110

Preparation of a nanoparticle pharmaceutical composition comprising albumin and Example 68: 100 μL of a human albumin solution (100 mg/mL in H₂O) was added to a 1.5 mL microcentrifuge tube and diluted with 500 μL of water and 100 μL of 10 mmol NaHCO₃(aq.). After vortexing for 5 seconds, 100 μL of a 30 mg/mL DMSO solution of Example 68 was added quickly to the albumin solution and immediately vortexed for 10-15 seconds. The crude nanoparticle solution was then purified by size exclusion chromatography (GE Health Sciences™ PD-10) and the particle size (Analysis by Number, Malvern Nano ZS) was determined to be 94 nm after 2 hours following purification.

### Example 111

Preparation of a nanoparticle pharmaceutical composition comprising albumin and Example 70: 100 μL of a human albumin solution (100 mg/mL in H₂O) was added to a 1.5 mL microcentrifuge tube and diluted with 500 μL of water and 100 μL of 10 mmol NaHCO₃(aq.). After vortexing for 5 seconds, 100 μL of a 30 mg/mL DMSO solution of Example 70 was added quickly to the albumin solution and immediately vortexed for 10-15 seconds. The crude nanoparticle solution was then purified by size exclusion chromatography (GE Health Sciences™ PD-10) and the particle size (Analysis by Number, Malvern Nano ZS) was determined to be 60 nm after 2 hours following purification.

### Example 112

Preparation of a nanoparticle pharmaceutical composition comprising albumin and Example 99: 100 μL of a human albumin solution (100 mg/mL in H₂O) was added to a 1.5 mL microcentrifuge tube and diluted with 500 μL of water and 100 μL of 10 mmol NaHCO₃(aq.). After vortexing for 5 seconds, 100 μL of a 30 mg/mL DMSO solution of Example 99 was added quickly to the albumin solution and immediately vortexed for 10-15 seconds. The crude nanoparticle solution was then purified by size exclusion chromatography (GE Health Sciences™ PD-10) and the particle size (Analysis by Number, Malvern Nano ZS) was determined to be 47 nm after 2 hours following purification. The purified nanoparticle solution was diluted with a 20 mM solution of sodium N-acetyl-DL-trytophanate and sodium caprylate and lyophilized. The particle size of the lyophilized material after being reconstituted in water was 47 nm.

### Example 113

Preparation of a nanoparticle pharmaceutical composition comprising albumin and Example 101: 100 μL of a human albumin solution (100 mg/mL in H₂O) was added to a 1.5 mL microcentrifuge tube and diluted with 500 μL of water and 100 μL of 10 mmol NaHCO₃(aq.). After vortexing for 5 seconds, 100 μL of a 30 mg/mL DMSO solution of Example 101 was added quickly to the albumin solution and immediately vortexed for 10-15 seconds. The crude nanoparticle solution was then purified by size exclusion chromatography (GE Health Sciences™ PD-10) and the particle size (Analysis by Number, Malvern Nano ZS) was determined to be 90 nm after 2 hours following purification.

### Example 114

Preparation of a nanoparticle pharmaceutical composition comprising albumin and Example 104: 100 μL of a human albumin solution (100 mg/mL in H₂O) was added to a 1.5 mL microcentrifuge tube and diluted with 500 μL of water and 100 μL of 10 mmol NaHCO₃(aq.). After vortexing for 5 seconds, 100 μL of a 30 mg/mL DMSO solution of Example 104 was added quickly to the albumin solution and immediately vortexed for 10-15 seconds. The crude nanoparticle solution was then purified by size exclusion chromatography (GE Health Sciences™ PD-10) and the particle size (Analysis by Number, Malvern Nano ZS) was determined to be 54 nm after 2 hours following purification.

### Example 115

Preparation of a nanoparticle pharmaceutical composition comprising albumin and Example 107: 100 μL of a human albumin solution (100 mg/mL in H₂O) was added to a 1.5 mL microcentrifuge tube and diluted with 500 μL of water and 100 μL of 10 mmol NaHCO₃(aq.). After vortexing for 5 seconds, 100 μL of a 30 mg/mL DMSO solution of Example 107 was added quickly to the albumin solution and immediately vortexed for 10-15 seconds. The crude nanoparticle solution was then purified by size exclusion chromatography (GE Health Sciences™ PD-10) and the particle size (Analysis by Number, Malvern Nano ZS) was determined to be 130 nm after 2 hours following purification.

### Example A

### Bcl-2 Protein Family Binding Assay

Binding to Bcl-2 proteins Bcl-2, and Bcl-X_{L} was assessed using the Bcl2scan™ platform: T7 phage strains displaying BCL2 proteins were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 m) to remove cell debris. Streptavidin-coated magnetic beads were treated with biotinylated BIM peptide ligand for 30 minutes at room temperature to generate affinity resins for BCL2 assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining BCL2 proteins, liganded affinity beads, and test compounds in 1× binding buffer (20% SeaBlock, 0.17×PBS, 0.05% Tween 20, 6 mM DTT). Test compounds were prepared as 100× stocks in 100% DMSO. Kds were determined using an 11-point 3-fold compound dilution series with one DMSO control point. All compounds for Kd measurements were distributed by acoustic transfer in 100% DMSO. The compounds were then diluted directly into the assays such that the final concentration of DMSO was 0.9%. All reactions performed in polypropylene 384-well plates. Each was a final volume of 0.02 ml. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 2 μM non-biotinylated affinity ligand) and incubated at room temperature with shaking for 30 minutes. The BCL2 concentration in the eluates was measured by qPCR. Binding constants (Kds) were calculated with a standard dose-response curve using the Hill equation:

$$Response = Background + \frac{Signal - Background}{1 + ( K {d}^{HillSlope} / {Dose}^{Hill Slope}} .$$

The Hill Slope was set to −1. Curves were fitted using a non-linear least square fit with the Levenberg-Marquardt algorithm. The results are shown in Table 1.

TABLE 1

| Example | Bcl-2 Kd (nm) | Bcl-X_{L} Kd (nm) |
|---|---|---|
| 1 | A | C |
| 9 | A | C |
| 10 | A | C |
| 11 | A | C |
| 13 | A | C |
| 15 | A | C |
| 16 | A | C |
| 17 | A | B |
| 20 | A | C |
| 23 | A | C |
| 25 | A | C |
| 26 | A | C |
| 27 | A | C |
| 28 | A | C |
| 30 | A | C |
| 31 | A | C |
| 32 | A | C |
| 33 | A | C |
| 34 | A | C |
| 35 | A | C |
| 36 | A | B |
| 38 | A | B |
| 39 | B | C |
| 40 | A | C |
| 47 | C | B |
| 48 | B | B |
| ABT-199 | A | B |
| ABT-263 | B | B |

Bc1-2 Binding Assay (Kd): A = a single Kd ≤10 nM; B = a single Kd >10 nM and <100 nM; C = a single Kd ≥100 nM

### Example B

### Bcl-2/Bcl-X_{L} Homogeneous Time Resolved Fluorescence (HTRF) Assay

Binding to Bcl-2 proteins Bcl-2, and Bcl-X_{L} was also assessed using an HTRF assay. Background: FAM-Bak/Bad binds to surface pocket of the Bcl-2 protein family. This binding can be monitored by HTRF signals between anti-GST-Tb and FAM-peptide using GST-tagged Bcl proteins. Assay conditions: Bcl-2: 4 nM Bcl-2, 100 nM FAM-Bak peptide, Bcl-X_{L}: 3 nM Bcl-X_{L}, 40 nM FAM-Bad peptide in 20 mM K Phosphate, pH 7.5, 50 mM NaCl, 1 mM EDTA, 0.005% Triton X-100 and 1% DMSO (final). Assay procedure: Compounds were tested in 10-dose IC₅₀ mode, in singlicate, with 3-fold serial dilution starting at 10 μM or 1 μM. Compound stock solutions were added to protein solution using Acoustic technology. The compounds were then incubated with protein for 10 min at room temperature. The respective FAM labeled peptide was added and incubated for another 10 min and then anti-GST-Tb was added. After 60 min at rt, the HTRF fluorescence signal ratio was measured. Curve fits were performed in GraphPad Prism 4 with “sigmoidal dose-response (variable slope)”; 4 parameters with Hill Slope. The results are shown in Table 2.

TABLE 2

| Example | Bcl-2 IC₅₀ (nM) | Bcl-X_{L} IC₅₀ (nM) |
|---|---|---|
| 15 | A | C |
| 20 | A | C |
| 34 | A | B |
| 46 | A | A |
| 48 | A | A |
| 49 | A | A |
| 50 | A | A |
| 55 | A | A |
| 58 | A | A |
| 59 | A | A |
| 60 | A | A |
| 61 | A | A |
| 62 | A | A |
| 63 | A | A |
| 64 | A | A |
| 67 | A | A |
| 69 | A | A |
| 70 | A | A |
| 98 | A | A |
| 99 | A | A |
| 100 | A | A |
| 104 | A | A |
| ABT-199 | A | B |
| ABT-263 | A | A |

Bcl-2 Binding Assay (IC₅₀): A = a single IC₅₀ ≤10 nM; B = a single IC₅₀ >10 nM and <100 nM; C = a single IC₅₀ >100 nM.

### Example C

### RS4;11, NCI-H1963, and NCI-H146 Cell Proliferation Assays

Cell proliferation was measured using the CellTiter-Glo® Luminescent Cell Viability Assay. The assay involved the addition of a single reagent (CellTiter-Glo® Reagent) directly to cells cultured in serum-supplemented medium. RS4;11 (ATCC, CRL-1873) cells were cultured according to ATCC recommendations and were seeded at 50,000 cells per well. NCI-H1963 cells (ATCC CRL-5982) were cultured according to ATCC recommendations and seeded at 12,000 cells per well. NCI-H146 (ATCC, HTB-173) cells were cultured according to ATCC recommendations and were seeded at 20,000 cells per well.

Each compound evaluated was prepared as a DMSO stock solution (10 mM). Compounds were tested in duplicate on each plate, with a 10-point serial dilution curve (1:3 dilution). Compound treatment (1.0 μL for RS4;11 and NCI-H1963, 10 μL for NCI-H146) was added from the compound dilution plate to the cell plate. The highest compound concentration was 10 μM (final), with a 0.1% final DMSO concentration. Plates were then incubated at 37° C., 5% CO₂. After 48 h of compound treatment for RS4;11 or 72 h for NCI-H-1963 and NCI-H-146, cell plates were equilibrated at rt for approximately 30 mins. An equi-volume amount of CellTiter-G)® Reagent (40 (L for RS4;191 and NC-H1963, 100 μL for NCI-H-146)) was added to each well. Plates were mixed for 2 mins on an orbital shaker to induce cell lysis and then incubated at RT for 10 mins to stabilize the luminescent signal. Luminescence was recorded using a Envision or SpectraMax M5e plate reader according to CellTiter-Glo protocol. IC₅₀ of each compound was calculated using GraphPad Prism by nonlinear regression analysis. IC₅₀ values are provided in Table 3.

TABLE 3

| Example# | RS4; 11 (nM) | H1963 (nM) | H146 (nm) |
|---|---|---|---|
| 1 | A | | |
| 2 | A | | |
| 3 | B | | |
| 4 | B | | |
| 5 | B | | |
| 6 | C | | |
| 7 | C | | |
| 8 | B | | |
| 9 | A | | |
| 10 | A | | |
| 11 | A | | |
| 12 | A | | |
| 13 | A | | |
| 14 | A | | |
| 15 | A | | |
| 16 | A | | |
| 17 | A | | |
| 18 | A | | |
| 19 | A | | |
| 20 | A | | |
| 21 | B | | |
| 22 | A | | |
| 23 | A | | |
| 24 | A | | |
| 25 | A | | |
| 26 | A | | |
| 27 | A | | |
| 28 | A | | |
| 29 | A | | |
| 30 | A | | |
| 31 | A | | |
| 32 | A | | |
| 33 | A | | |
| 34 | A | | |
| 35 | A | | |
| 36 | A | | |
| 37 | A | | |
| 38 | A | | |
| 39 | A | | |
| 40 | A | | |
| 42 | A | | |
| 43 | B | | |
| 45 | C | | |
| 46 | B | | |
| 47 | C | | |
| 48 | B | | |
| 49 | B | | |
| 50 | B | | |
| 51 | B | | |
| 52 | B | | |
| 53 | C | | |
| 54 | C | | |
| 55 | B | | |
| 56 | C | B | |
| 57 | C | C | |
| 58 | B | | |
| 59 | B | B | |
| 60 | A | B | |
| 61 | A | A | |
| 62 | B | A | |
| 63 | A | A | |
| 64 | B | C | |
| 65 | | B | |
| 67 | B | | |
| 68 | B | A | |
| 69 | | B | |
| 70 | B | C | |
| 71 | | B | |
| 72 | C | | |
| 98 | | | B |
| 99 | | | A |
| 100 | | | A |
| 101 | | | A |
| 102 | | | A |
| 103 | | | B |
| 104 | | | A |
| 105 | | | A |
| 106 | | | B |
| 107 | | | A |
| 108 | | | A |
| ABT-199 | A | C | |
| ABT-263 | A | A | A |

For RS4; 11, H146 CTG IC₅₀:
A = a single IC₅₀ ≤ 100 nM;
B = a single IC₅₀ > 100 nM and < 1000 nM;
C = a single IC₅₀ ≥ 1000 nM.
For H1963 CTG IC₅₀:
A = a single IC₅₀ ≤ 500 nM;
B = a single IC₅₀ > 500 nM and < 1000 nM;
C = a single IC₅₀ ≥ 1000 nM.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A pharmaceutical composition comprising a compound of Formula (I), or a pharmaceutically acceptable salt thereof, and a pharmaceutically acceptable carrier comprising albumin, wherein the compound of Formula (I) has the structure: 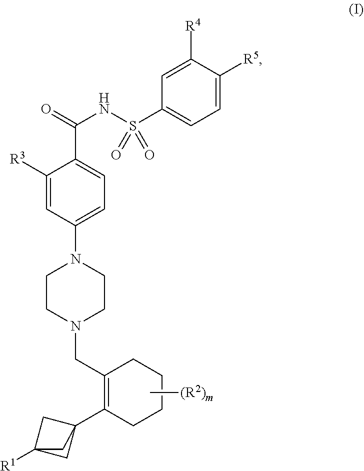
wherein:
R¹ is selected from the group consisting of hydrogen, halogen, a substituted or unsubstituted C₁-C₆ alkyl, a substituted or unsubstituted C₁-C₆ haloalkyl, a substituted or unsubstituted C₃-C₆ cycloalkyl, a substituted or unsubstituted C₁-C₆ alkoxy, an unsubstituted mono-C₁-C₆ alkylamino and an unsubstituted di-C₁-C₆ alkylamino;
each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl; or
when m is 2 or 3, each R² is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl, or alternatively, two R² groups together with the atom(s) to which they are attached form a substituted or unsubstituted C₃-C₆ cycloalkyl or a substituted or unsubstituted 3 to 6 membered heterocyclyl;
R³ is hydrogen or halogen;
R⁴ is selected from the group consisting of —NO₂, —S(O)R⁶, —S(O)₂R⁶, halogen, cyano and an unsubstituted C₁-C₆ haloalkyl;
R⁵ is —X¹-(Alk¹)ₙ-R⁷ or —X²(CHR⁸)-(Alk²)ₚ-X³-R⁹;
Alk¹ and Alk² are each independently an unsubstituted C₁-C₄ alkylene or a C₁-C₄ alkylene substituted with 1, 2 or 3 substituents independently selected from the group consisting of fluoro, chloro, an unsubstituted C₁-C₃ alkyl and an unsubstituted C₁-C₃ haloalkyl;
R⁶ is selected from the group consisting of a substituted or unsubstituted C₁-C₆alkyl, a substituted or unsubstituted C₁-C₆ haloalkyl, and a substituted or unsubstituted C₃-C₆ cycloalkyl;
R⁷ is selected from the group consisting of a substituted or unsubstituted C₁-C₆alkoxy, a substituted or unsubstituted C₃-C₁₀ cycloalkyl, a substituted or unsubstituted 3 to 10 membered heterocyclyl, hydroxy, amino, a substituted or unsubstituted mono-substituted amino group, a substituted or unsubstituted di-substituted amino group, a substituted or unsubstituted N-carbamyl, a substituted or unsubstituted C-amido and a substituted or unsubstituted N-amido;
R⁸ is selected from the group consisting of a substituted or unsubstituted 3 to 10 membered heterocyclyl(C₁-C₆ alkyl), a substituted or unsubstituted di-C₁-C₆ alkylamino(C₁-C₆ alkyl) and a substituted or unsubstituted mono-C₁-C₆ alkylamino(C₁-C₆ alkyl);
R⁹ is a substituted or unsubstituted 5 to 10 membered heteroaryl or a substituted or unsubstituted C₆-C₁₀ aryl;
m is 0, 1, 2 or 3;
n and p are each independently 0 or 1; and
X, X¹, X² and X³ are each independently —O—, —S— or —NH—.

2. The pharmaceutical composition of claim 1, wherein R¹ is halogen.

3. The pharmaceutical composition of claim 1 or 2, wherein R¹ is fluoro.

4. The pharmaceutical composition of claim 1 or 2, wherein R¹ is chloro.

5. The pharmaceutical composition of claim 1, wherein R¹ is a substituted or unsubstituted C₁-C₆ alkyl.

6. The pharmaceutical composition of claim 1 or 5, wherein R¹ is an unsubstituted C₁-C₆ alkyl.

7. The pharmaceutical composition of any one of claim 1 or 5-6, wherein R¹ is an unsubstituted methyl or an unsubstituted ethyl.

8. The pharmaceutical composition of claim 1, wherein R¹ is a substituted or unsubstituted C₁-C₆ haloalkyl.

9. The pharmaceutical composition of claim 1 or 8, wherein R¹ is an unsubstituted —CHF₂, —CF₃, —CH₂CF₃, —CF₂CF₃ or —CF₂CH₃.

10. The pharmaceutical composition of claim 1, wherein R¹ is hydrogen.

11. The pharmaceutical composition of claim 1, wherein R¹ is a substituted or unsubstituted C₃-C₆ cycloalkyl.

12. The pharmaceutical composition of claim 1 or 11, wherein R¹ is an unsubstituted C₃-C₆ cycloalkyl.

13. The pharmaceutical composition of claim 1, wherein R¹ is a substituted or unsubstituted C₁-C₆ alkoxy.

14. The pharmaceutical composition of claim 1 or 13, wherein R¹ is an unsubstituted C₁-C₆ alkoxy.

15. The pharmaceutical composition of any one of claim 1 or 13-14, wherein R¹ is an unsubstituted methoxy or an unsubstituted ethoxy.

16. The pharmaceutical composition of claim 1, wherein R¹ is an unsubstituted mono-C₁-C₆ alkylamino.

17. The pharmaceutical composition of claim 1 or 16, wherein R¹ is methylamino or ethylamino.

18. The pharmaceutical composition of claim 1, wherein R¹ is an unsubstituted di-C₁-C₆ alkylamino.

19. The pharmaceutical composition of claim 1 or 18, wherein R¹ is di-methylamino or di-ethylamino.

20. The pharmaceutical composition of any one of claims 1-19, wherein m is 1.

21. The pharmaceutical composition of any one of claims 1-19, wherein m is 2.

22. The pharmaceutical composition of any one of claims 1-19, wherein m is 3.

23. The pharmaceutical composition of any one of claims 1-22, wherein one R² is an unsubstituted C₁-C₆ alkyl and each other R², if present, is independently selected from the group consisting of halogen, a substituted or unsubstituted C₁-C₆ alkyl, a substituted or unsubstituted C₁-C₆ haloalkyl and a substituted or unsubstituted C₃-C₆ cycloalkyl.

24. The pharmaceutical composition of any one of claims 1-22, wherein each R² is independently an unsubstituted C₁-C₆ alkyl.

25. The pharmaceutical composition of any one of claims 1-19 or 21, wherein m is 2, and wherein each R² is an unsubstituted methyl.

26. The pharmaceutical composition of any one of claims 1-19 or 21-22, wherein two R² groups together with the atom(s) to which they are attached form a substituted or unsubstituted C₃-C₆ cycloalkyl.

27. The pharmaceutical composition of any one of claims 1-19, 21-22 or 26, wherein two R² groups together with the atom(s) to which they are attached form an unsubstituted cyclopropyl or an unsubstituted cyclobutyl.

28. The pharmaceutical composition of any one of claims 1-19 or 21-22, wherein two R² groups together with the atom(s) to which they are attached form a substituted or unsubstituted 3 to 6 membered heterocyclyl.

29. The pharmaceutical composition of any one of claims 1-19, wherein m is 0.

30. The pharmaceutical composition of any one of claims 1-19, wherein the structure of Formula (I) is also represented by Formula (Ia), Formula (Ib), Formula (Ic), Formula (Id), Formula (Ie), or Formula (If):

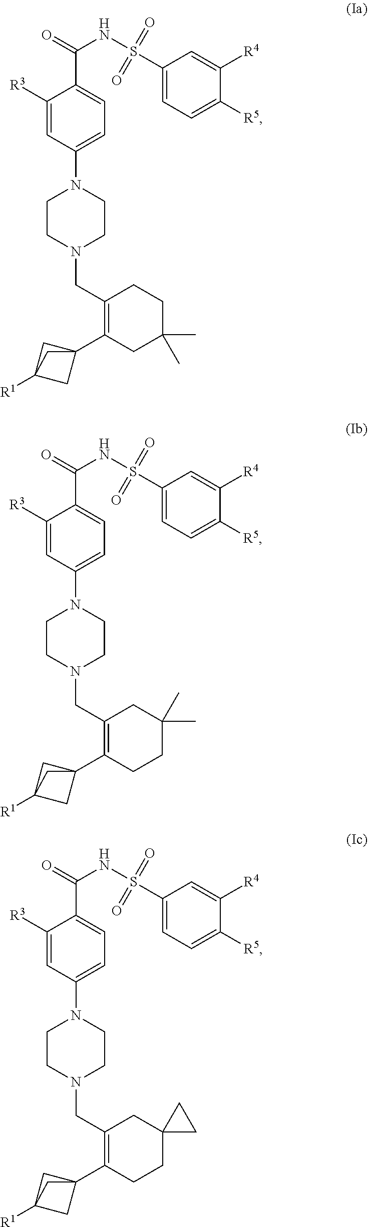

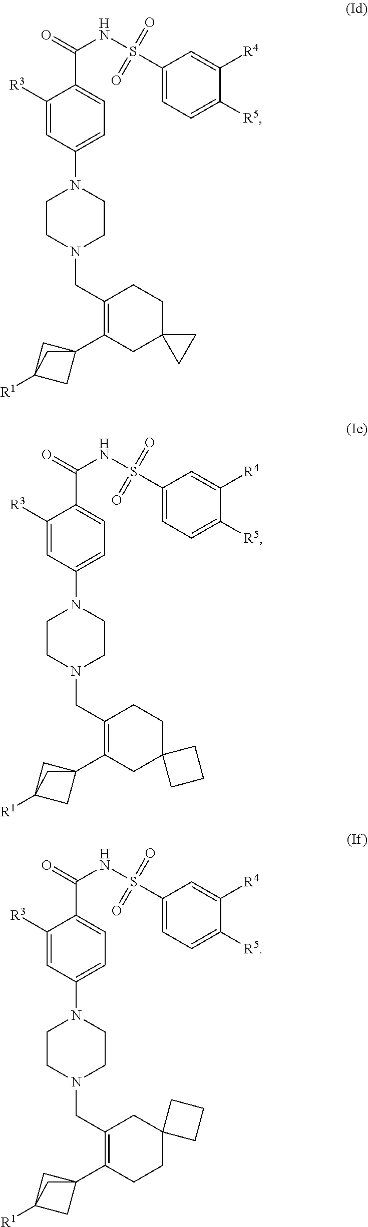

31. The pharmaceutical composition of any one of claims 1-30, wherein R³ is hydrogen.

32. The pharmaceutical composition of any one of claims 1-30, wherein R³ is halogen.

33. The pharmaceutical composition of any one of claims 1-32, wherein R⁴ is —NO₂.

34. The pharmaceutical composition of any one of claims 1-32, wherein R⁴ is cyano.

35. The pharmaceutical composition of any one of claims 1-32, wherein R⁴ is halogen.

36. The pharmaceutical composition of any one of claims 1-32, wherein R⁴ is an unsubstituted C₁-C₆ haloalkyl.

37. The pharmaceutical composition of any one of claims 1-32 or 36, wherein R⁴ is —CF₃.

38. The pharmaceutical composition of any one of claims 1-32, wherein R⁴ is —S(O)R⁶.

39. The pharmaceutical composition of any one of claims 1-32, wherein R⁴ is —S(O)₂R⁶.

40. The pharmaceutical composition of any one of claims 1-32 or 38-39, wherein R⁶ is a substituted or unsubstituted C₁-C₆ alkyl.

41. The pharmaceutical composition of any one of claims 1-32 or 38-39, wherein R⁶ is a substituted or unsubstituted C₃-C₆ cycloalkyl.

42. The pharmaceutical composition of any one of claims 1-32 or 38-39, wherein R⁶ is a substituted or unsubstituted C₁-C₆ haloalkyl.

43. The pharmaceutical composition of any one of claims 38-39 or 42, wherein R⁶ is —CF₃.

44. The pharmaceutical composition of any one of claims 1-43, wherein R⁵ is —X¹-(Alk¹)ₙ-R⁷.

45. The pharmaceutical composition of any one of claims 1-44, wherein X¹ is —O—.

46. The pharmaceutical composition of any one of claims 1-44, wherein X¹ is —S—.

47. The pharmaceutical composition of any one of claims 1-44, wherein X¹ is —NH—.

48. The pharmaceutical composition of any one of claims 1-47, wherein Alk¹ is unsubstituted —(CH₂)₁₋₄—* for which “*” represents the point of attachment to R⁷.

49. The pharmaceutical composition of any one of claims 1-47, wherein Alk¹ is selected from the group consisting of 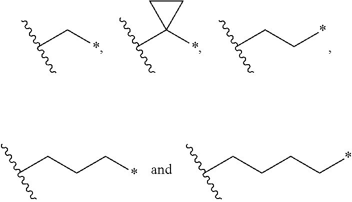
for which “*” represents the point of attachment to R⁷.

50. The pharmaceutical composition of any one of claims 1-47, wherein Alk¹ is a substituted 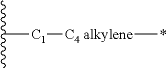
for which “*” represents the point of attachment to R⁷.

51. The pharmaceutical composition of any one of claims 1-47 or 50, wherein Alk¹ is selected from the group consisting of

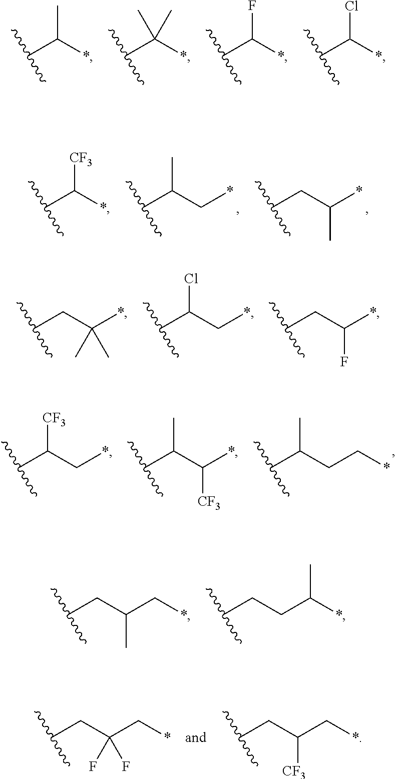

52. The pharmaceutical composition of any one of claims 1-51, wherein n is 1.

53. The pharmaceutical composition of any one of claims 1-47, wherein n is 0.

54. The pharmaceutical composition of any one of claims 1-53, wherein R⁷ is a substituted or unsubstituted mono-substituted amino group.

55. The pharmaceutical composition of any one of claims 1-53, wherein R⁷ is a substituted or unsubstituted di-substituted amino group.

56. The pharmaceutical composition of any one of claims 1-53, wherein R⁷ is a substituted or unsubstituted N-carbamyl, a substituted or unsubstituted C-amido or a substituted or unsubstituted N-amido.

57. The pharmaceutical composition of any one of claims 1-53, wherein R⁷ is a substituted or unsubstituted C₃-C₁₀ cycloalkyl.

58. The pharmaceutical composition of any one of claims 1-53 or 57, wherein R⁷ is a substituted or unsubstituted C₆-C₁₀ spiro cycloalkyl.

59. The pharmaceutical composition of any one of claims 1-53, wherein R⁷ is a substituted or unsubstituted 3 to 10 membered heterocyclyl.

60. The pharmaceutical composition of any one of claims 1-53 or 59, wherein R⁷ is a substituted or unsubstituted 6 to 10 membered spiro heterocyclyl.

61. The pharmaceutical composition of any one of claims 1-53, wherein R⁷ is hydroxy or amino.

62. The pharmaceutical composition of any one of claims 1-61, wherein R⁷ is unsubstituted.

63. The pharmaceutical composition of any one of claims 1-60, wherein R⁷ is substituted.

64. The pharmaceutical composition of any one of claims 1-60 or 63, wherein R⁷ is substituted with 1 or 2 substituents independently selected from the group consisting of an unsubstituted C₁-C₆ alkyl, an unsubstituted C₁-C₆ alkoxy, fluoro, chloro, hydroxy and —S(O)₂-(unsubstituted C₁-C₆ alkyl).

65. The pharmaceutical composition of any one of claims 1-53, wherein R⁷ is selected from the group consisting of

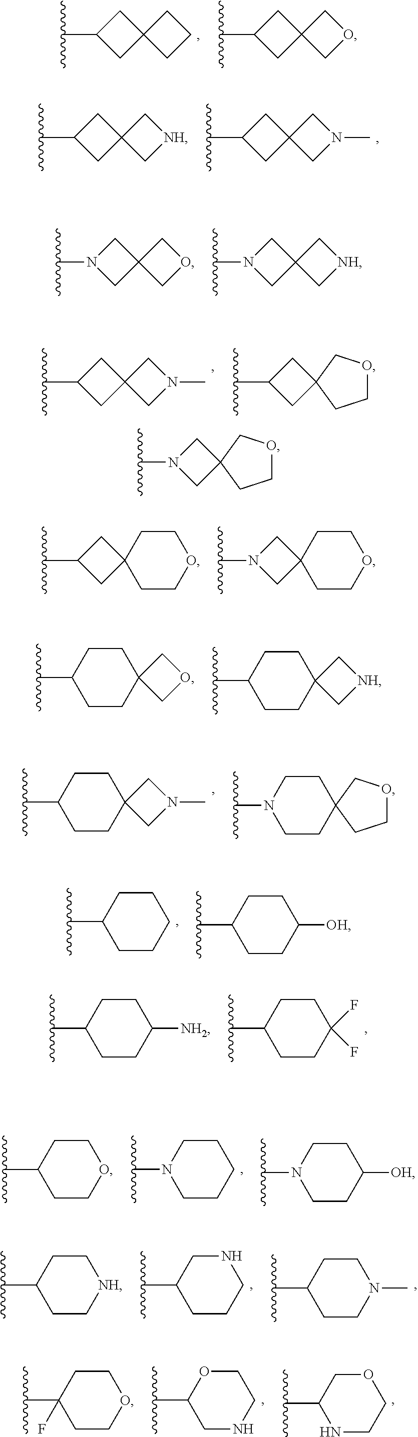

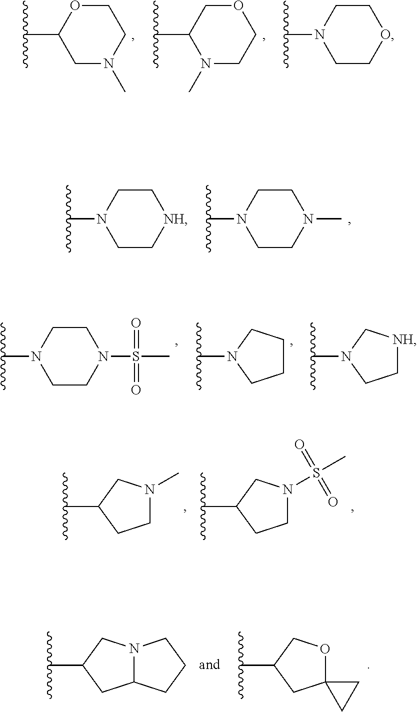

66. The pharmaceutical composition of any one of claims 1-53, wherein R⁷ is selected from the group consisting of

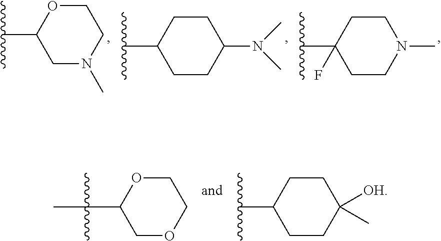

67. The pharmaceutical composition of any one of claims 1-43, wherein R⁵ is —X²—(CHR⁸)-(Alk²)ₚ-X³-R⁹.

68. The pharmaceutical composition of any one of claims 1-43 or 67, wherein X² is —O—.

69. The pharmaceutical composition of any one of claims 1-43 or 67, wherein X² is —S—.

70. The pharmaceutical composition of any one of claims 1-43 or 67, wherein X² is —NH—.

71. The pharmaceutical composition of any one of claims 1-43 or 67-70, wherein X³ is —O—.

72. The pharmaceutical composition of any one of claims 1-43 or 67-70, wherein X³ is —S—.

73. The pharmaceutical composition of any one of claims 1-43 or 67-70, wherein X³ is —NH—.

74. The pharmaceutical composition of any one of claims 1-43 or 67-73, wherein Alk² is unsubstituted —(CH₂)₁₋₄—* for which “*” represents the point of attachment to X³.

75. The pharmaceutical composition of any one of claims 1-43 or 67-73, wherein Alk² is 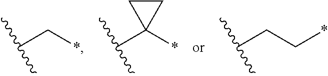
for which “*” represents the point of attachment to X³.

76. The pharmaceutical composition of any one of claims 1-43 or 67-73, wherein Alk² is a substituted 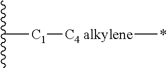
for which “*” represents the point of attachment to X³.

77. The pharmaceutical composition of any one of claims 1-40, 67-74 or 76, wherein Alk² is selected from the group consisting of

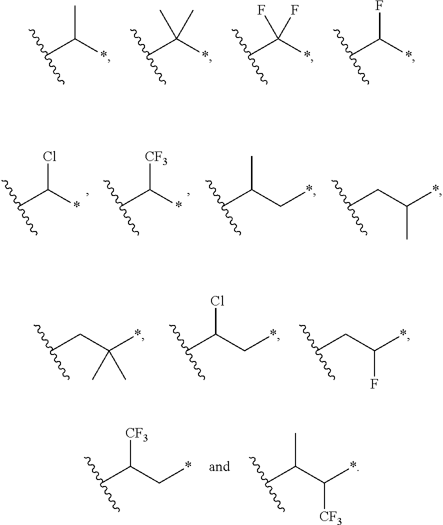

78. The pharmaceutical composition of any one of claims 1-43 or 67-77, wherein p is 1.

79. The pharmaceutical composition of any one of claims 1-43 or 67-73, wherein p is 0.

80. The pharmaceutical composition of any one of claims 1-43 or 67-79, wherein R⁸ is a substituted or unsubstituted 3 to 10 membered heterocyclyl(C₁-C₆ alkyl).

81. The pharmaceutical composition of any one of claims 1-43 or 67-80, wherein R⁸ is a substituted or unsubstituted 6 to 10 membered spiro heterocyclyl(C₁-C₆ alkyl).

82. The pharmaceutical composition of any one of claims 1-43 or 67-79, wherein R⁸ is a substituted or unsubstituted di-C₁-C₆ alkylamino(C₁-C₆ alkyl).

83. The pharmaceutical composition of any one of claims 1-43, 67-79 or 82, wherein R⁸ is a substituted or unsubstituted di-methylamino(C₁-C₆ alkyl).

84. The pharmaceutical composition of any one of claims 1-43 or 67-79, wherein R⁸ is a substituted or unsubstituted mono-C₁-C₆ alkylamino(C₁-C₆ alkyl).

85. The pharmaceutical composition of any one of claims 1-43 or 67-84, wherein R⁸ is substituted.

86. The pharmaceutical composition of any one of claims 1-43 or 67-85, wherein R⁸ is substituted with 1 or 2 substituents independently selected from the group consisting of an unsubstituted C₁-C₆ alkyl, an unsubstituted C₁-C₆ alkoxy, an unsubstituted di-C₁-C₆ alkylamino, an unsubstituted acyl(C₁-C₆ alkyl), an unsubstituted C-carboxy, fluoro, chloro and hydroxy.

87. The pharmaceutical composition of any one of claims 1-43 or 67-84, wherein R⁸ is unsubstituted.

88. The pharmaceutical composition of any one of claims 1-43 or 67-87, wherein R⁸ is selected from the group consisting of

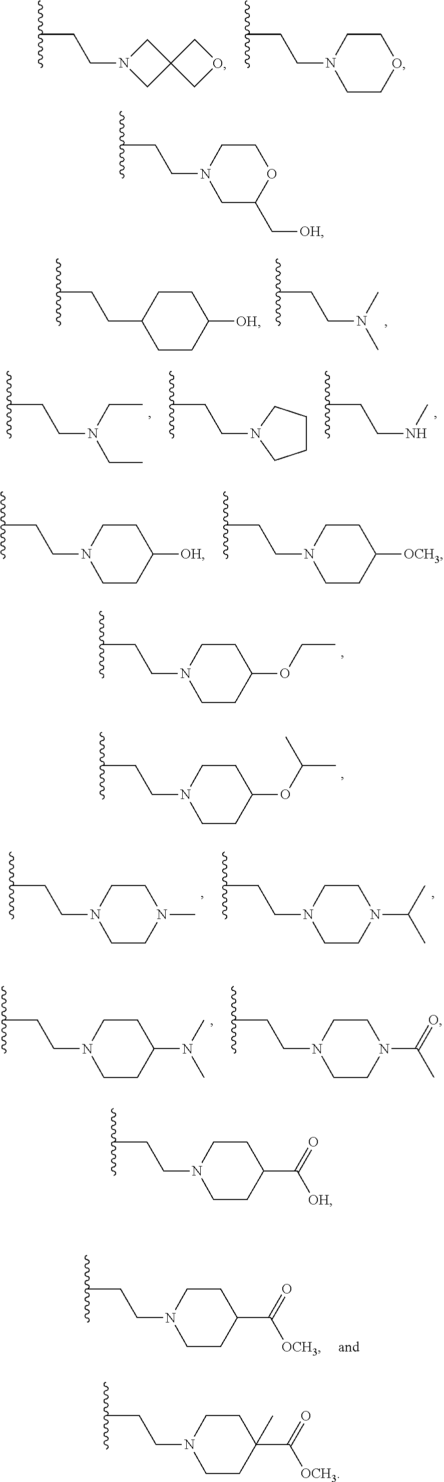

89. The pharmaceutical composition of any one of claims 1-43 or 67-87, wherein R⁸ is selected from the group consisting of

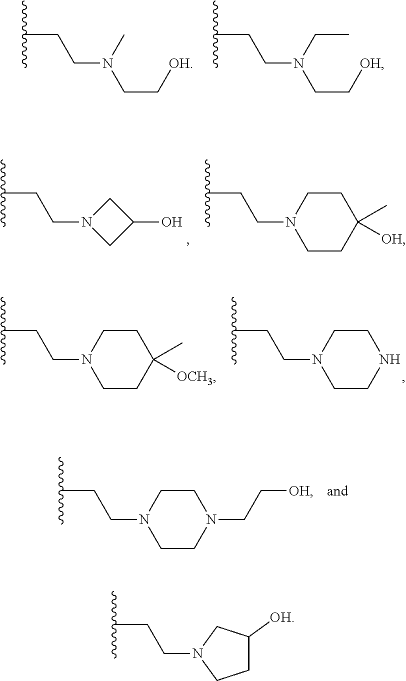

90. The pharmaceutical composition of any one of claims 1-43 or 67-89, wherein R⁹ is a substituted or unsubstituted C₆-C₁₀ aryl.

91. The pharmaceutical composition of any one of claims 1-43 or 67-90, wherein R⁹ is an unsubstituted C₆-C₁₀ aryl.

92. The pharmaceutical composition of any one of claims 1-43 or 67-91, wherein R⁹ is an unsubstituted phenyl.

93. The pharmaceutical composition of any one of claims 1-43 or 67-89, wherein R⁹ is a substituted or unsubstituted 5 to 10 membered heteroaryl.

94. The pharmaceutical composition of claim 1, wherein the compound is listed in FIG. 1 of this application.

95. The pharmaceutical composition of claim 94, wherein the compound of Formula (I) is selected from the group consisting of 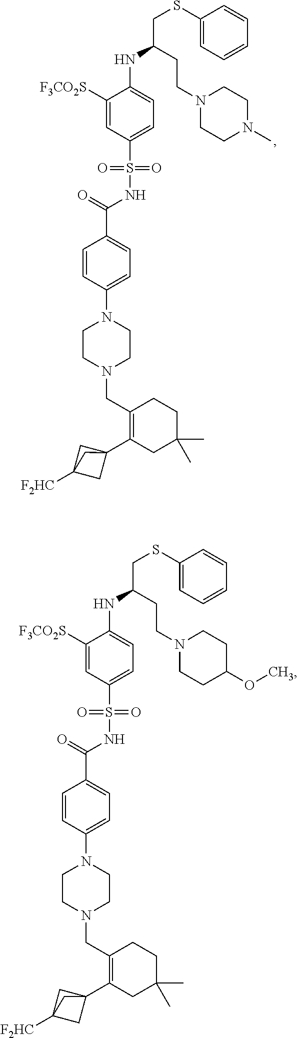 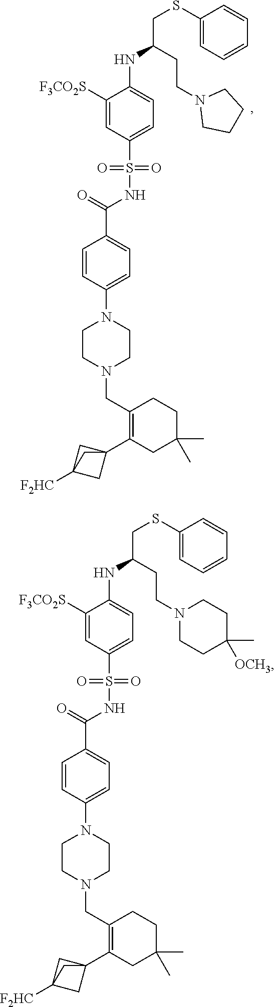 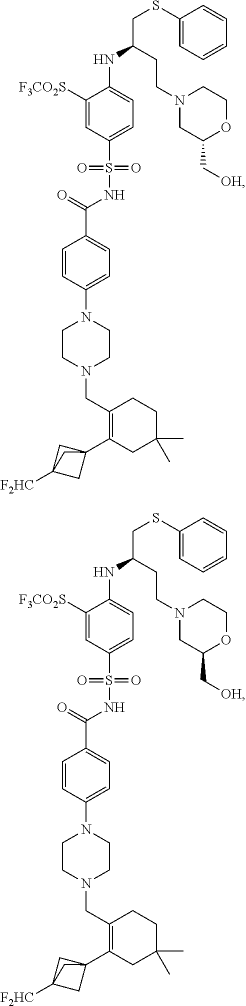 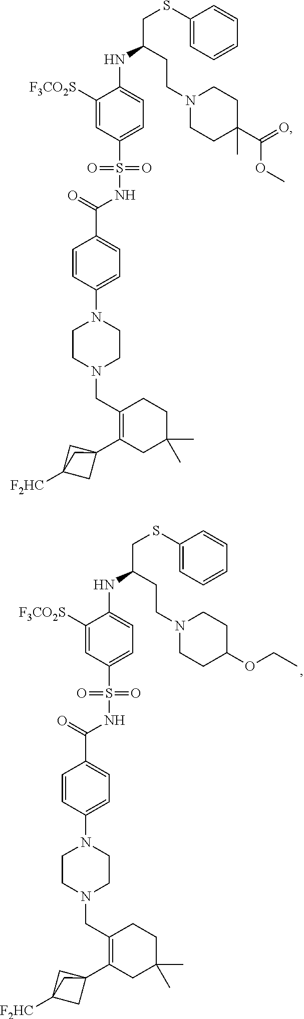 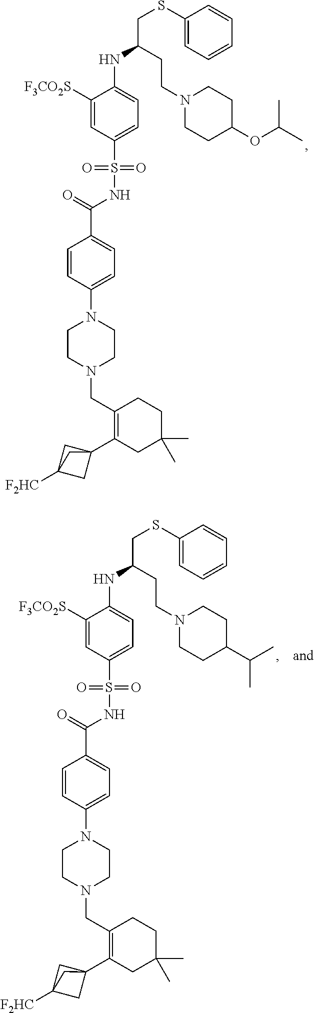 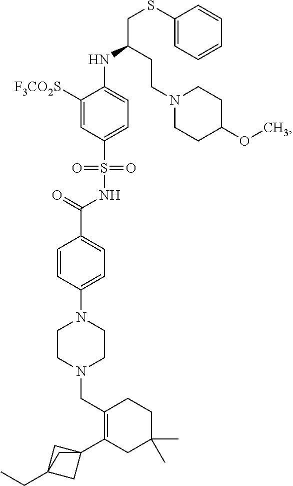
or a pharmaceutically acceptable salt thereof.

96. The pharmaceutical composition of any one of claims 1-95, wherein the compound of Formula (I) is a Bcl-2 inhibitor.

97. The pharmaceutical composition of any one of claims 1-96, wherein the compound of Formula (I) is a dual Bcl-2/xL inhibitor.

98. The pharmaceutical composition of any one of claims 1-97, wherein the albumin is human serum albumin or bovine serum albumin.

99. The pharmaceutical composition of any one of claims 1-98, wherein the albumin is human serum albumin.

100. The pharmaceutical composition of any one of claims 1-99 that is free of surfactant.

101. The pharmaceutical composition of any one of claims 1-100, wherein the compound of Formula (I), or a pharmaceutically acceptable salt thereof, and the albumin in the pharmaceutical composition are formulated as particles.

102. The pharmaceutical composition of claim 101, wherein the particles have an average diameter of less than 10 μm, less than 1 μm, less than 800 nm, less than 500 nm, less than 200 nm, or less than 100 nm.

103. The pharmaceutical composition of any one of claims 1-102, wherein the ratio (w/w) of the albumin to the compound of Formula (I), or a pharmaceutically acceptable salt thereof, in the pharmaceutical composition is in a range from about 1:50 to about 100:1, from about 1:10 to about 100:1, from about 1:5 to about 100:1, from about 1:1 to about 100:1, from about 1:1 to about 90:1, from about 1:1 to about 80:1, from about 1:1 to about 70:1, from about 1:1 to about 60:1, or from about 1:1 to about 50:1.

104. The pharmaceutical composition of any one of claims 1-102, wherein the ratio (w/w) of the albumin to the compound of Formula (I), or a pharmaceutically acceptable salt thereof, in the pharmaceutical composition is in a range from 1:50 to 100:1, from 1:10 to 100:1, from 1:5 to 100:1, from 1:1 to 100:1, from 1:1 to 90:1, from 1:1 to 80:1, from 1:1 to 70:1, from 1:1 to 60:1, or from 1:1 to 50:1.

105. The pharmaceutical composition of any one of claims 1-102, wherein the ratio (w/w) of the albumin to the compound of Formula (I), or a pharmaceutically acceptable salt thereof, in the pharmaceutical composition is about 1:50, about 1:40, about 1:30, about 1:20, about 1:10, about 1:1, about 10:1, about 20:1, about 30:1, about 40:1, about 50:1, about 60:1, about 70:1, about 80:1, about 90:1 or about 100:1.

106. The pharmaceutical composition of any one of claims 1-102, wherein the ratio (w/w) of the albumin to the compound of Formula (I), or a pharmaceutically acceptable salt thereof, in the pharmaceutical composition is 1:50, 1:40, 1:30, 1:20, 1:10, 1:1, 10:1, 20:1, 30:1, 40:1, 50:1, 60:1, 70:1, 80:1, 90:1 or 100:1.

107. The pharmaceutical composition of any one of claims 1-106 that is formulated for intravenous administration.

108. The pharmaceutical composition of any one of claims 1-106 that is formulated for injection.

109. A method for treating a cancer or a tumor comprising administering an effective amount of the pharmaceutical composition of any one of claims 1-108, to a subject having the cancer or the tumor, wherein the cancer or the tumor is selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

110. A method for inhibiting replication of a malignant growth or a tumor comprising contacting the growth or the tumor with an effective amount of the pharmaceutical composition of any one of claims 1-108, wherein the malignant growth or tumor selected from an Ewings's tumor and a Wilm's tumor, or the malignant growth of tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma.

111. A method for treating a cancer comprising contacting a malignant growth or a tumor with an effective amount of the pharmaceutical composition of any one of claims 1-108, wherein the malignant growth or tumor selected from an Ewings's tumor and a Wilm's tumor, or the malignant growth of tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma or an osteosarcoma.

112. A method for inhibiting the activity of Bcl-2 comprising providing an effective amount of the pharmaceutical composition of any one of claims 1-108 to a cancer cell or a tumor, wherein the cancer cell or the tumor is from a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

113. A method for inhibiting the activity of Bcl-2 in a subject comprising providing an effective amount of the pharmaceutical composition of any one of claims 1-108 to the subject having a cancer or a tumor, wherein the cancer or the tumor is selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

114. Use of an effective amount of the pharmaceutical composition of any one of claims 1-108 in the manufacture of a medicament for treating a cancer or a tumor, wherein the cancer or the tumor is selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

115. Use of an effective amount of the pharmaceutical composition of any one of claims 1-108 in the manufacture of a medicament for inhibiting replication of a malignant growth or a tumor, wherein the malignant growth or the tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.

116. Use of an effective amount of the pharmaceutical composition of any one of claims 1-108 in the manufacture of a medicament for treating a malignant growth or a tumor, wherein the malignant growth or the tumor is due to a cancer selected from a bladder cancer, a brain cancer, a breast cancer, a bone marrow cancer, a cervical cancer, a colorectal cancer, an esophageal cancer, a hepatocellular cancer, a lymphoblastic leukemia, a follicular lymphoma, a lymphoid malignancy of T-cell or B-cell origin, a melanoma, a myelogenous leukemia, a Hodgkin's lymphoma, a Non-Hodgkin's lymphoma, a head and neck cancer (including oral cancer), an ovarian cancer, a non-small cell lung cancer, a chronic lymphocytic leukemia, a myeloma, a prostate cancer, a small cell lung cancer, a spleen cancer, a polycythemia vera, a thyroid cancer, an endometrial cancer, a stomach cancer, a gallbladder cancer, a bile duct cancer, a testicular cancer, a neuroblastoma, an osteosarcoma, an Ewings's tumor and a Wilm's tumor.
